SEQUENCE LISTING (94 sequences)

SEQ ID NO: 1 - protein, 132 aa, Homo sapiens
MSQGRHLLEFLPLYIAFMLRGVCRIDAGSLNPELFLPMLHEEDWCWEIAGHVDSQELFVGLFSSTSTGHAELDKKVNGLYYDSVFQLSLDRMRHTRSMARVERLRHRKAIQKKTQLVHHLLFKGWASDETEI

SEQ ID NO: 2 - DNA, 399 bp, Homo sapiens
atgtcacaaggcaggcatcttcttgagtttcttccattgtacatagctttcatgttacgtggggtttgtaggatagacgctggaagccttaatccagaactgtttttgccaatgttacatgaagaggattggtgttgggagatagctggccatgtggactcccaagagttattcgttggtttgttttctagtacctctactgggcatgcagagctggacaaaaaggttaatggactttattatgactctgtattccagttgtctctggaccgtatgcgtcatacaaggagtatggctagagtagagaggctgagacacaggaaagcgatccagaaaaagactcagttagtccatcatctgctatttaaaggatgggcttctgatgaaactgaaatttag

SEQ ID NO: 3 - protein, 147 aa, Homo sapiens
MGFIFSKSMNESMKNQKEFMLMNARLQLERQLIMQSEMRERQMAMQIAWSREFLKYFGTFFGLAAISLTAGAIKKKKPAFLVPIVPLSFILTYQYDLGYGTLLERMKGEAEDILETEKSKLQLPRGMITFESIEKARKEQSRFFIDK

SEQ ID NO: 4 - DNA, 932 bp, Homo sapiens
gagcgaggcccggtccctgcagcgggcgaaaggagcccgggcctggaggtttgcgtaccggtcgcctggtcccggcaccagcgccgcccagtgtggtttcccataaggaagctcttcttcctgcttggcttccacctttaacccttccacctgggagcgtcctctaacacattcagactacaagtccagacccaggagagcaaggcccagaaagaggtcaaaatggggtttatattttcaaaatctatgaatgaaagcatgaaaaatcaaaaggagttcatgcttatgaatgctcgacttcagctggaaaggcagctcatcatgcagagtgaaatgagggaaagacaaatggccatgcagattgcgtggtctcgggaattcctcaaatattttggaactttttttggccttgcagccatctctttaacagctggagcgattaaaaaaaagaagccagccttcctggtcccgattgttccattaagctttatcctcacctaccagtatgacttgggctatggaacccttttagaaagaatgaaaggtgaagctgaggacatactggaaacagaaaagagtaaattgcagctgccaagaggaatgatcacttttgaaagcattgaaaaagccagaaaggaacagagtagattcttcatagacaaatgaaatcatgcttaccaatcaaatctcaaagcacagaattattgacttgaatcatggtttttacagttttttaaatgctcaagattttgatattatagattttattttaaaatattaaaatgcaagatagttttgagctattttaaaataaaatttataacattcaacacaaaatcatggaggtgctctaaataacttttagatttcctctctctgtgtgcattaccaatatctaagtgtaaaattaataaattgttttgaattcctgg

SEQ ID NO: 5 - protein, 131 aa, Homo sapiens
MFLGLVGLRTKGRRWISSWSEGEDRGQSPEGVLLTWVFGTKCVMHPCEETTKQALCEQQGCLFHLGADELSPKRESAQSISFKWENSIYLHATLFLIGEYLHLAFYYFLLVLYILCSFLSYCLLLWLGSFL

SEQ ID NO: 6 - DNA, 2950 bp, Homo sapiens
attatctaggtctcggaggatggagaaatcaaaagtgccattttctggccatttagaaccattgtcgagtttgtattggggccaagcagtgttgcagaagaaaataagacatttagattttagttcaggtgatagttgaagaaattttaagttcttgagaacacaggctaagggagaagaaggaggaatggagggtggaagtttgcccatagtgaaggaggcaagtttaaagagaaaggtagagacatggagaaagggttggggagcagccctgggctgcaatgtgggtgagcagccaaagcaggcatccccgcaattgacttgccaccaagggaatgtggttgaatgaccaaggcaggcatccctgaagatatcagacgccaatggaatgtgggtgaataatcaggcaggcatccccggaatgattaaacactaagggaaggctgccttcctgagtacatgaccagcaccagagttttgggtccatggataaaatgtgtctcctttgtctctactagaaaatgaaaggaattgaaattaagagaagagagggagtgaagggtggcaccaagaatgaaaggagaaagaggttgagggatagtgagaaaggttggagaagagagtaaaaagaggccacttacccgatttaaaatttgtgagatgttccttgggctggttggtctgaggaccaaaggtcgtaggtggatctcttcatggagtgagggtgaggacaggggacagtctcctgaaggagtcctgctgacctgggtctttggcaccaaatgtgtcatgcatccatgtgaagagaccaccaaacaggctttgtgtgagcaacagggctgtttgtttcacctgggtgcagacgagttgagtccaaaaagagagtcagcccagtctatatcttttaaatgggaaaattcaatctatttacatgcaacgttattcttgataggtgagtacttacatctagcattctattattttctgcttgttttgtatatcctttgttccttcctctcttactgtttacttttgtggttgggcagttttctgtagggataagatttgagtcttatctctttctccctatgtgttagctttaccagtgagtttttatagtttcacatatttttatgatgctggttatcatcttctctgtggggaacaggccccccaaaacctggccataaactggccccaaaactggccataaacaaaatctctgcagcactgtgacatgtacatgatggtcttaacgcccacgctggaaggttgtgggtttaccagaatgagggcaaggaacacctggcccacccagggtggaaaaccgcttaaaggcattcttaaaccacaaacaatagcatgagtgatctgtgccttaaggccatgctcctgctgcagatagctagtccaacccatccctttatttcagcccatctcttcatttcccataaggaataattttagttaatctaatatctatagaaagaatgctaatgactagcttgctgttaataaatacatgggtaaacctctgttggaggctctcagctctgaaggctgtgagacccttgatttcctacttcactcctctatatttctgtgtctttaattcctctagtgccactgggttagagtctccccgaccaagctggtctcagcaagtggtctccatcatgggggctcgaatccaggttgaagggtcaccagagtgatggttggagaacatggaactagctggaggacacctgagtactcttaaagcaaaccccgtggtgagtaagaaggggagctcagaagcatcagggtaacaatgggacaagtgtggggtctggttcgttccatcttggaactttttcacactgatgatgaggaagaaggagagtataatgaagtaacagaagaggttatagagcaggtttatttgccagctaaagctaaagtggcaaaggagggagaggttcatccctacccttctgcaccccctcattattattttgaagaaaaagagtggcctgaccctccagatctttcttttccagaggacagtgggcaaaaattagttgccccagtgactgttcaagcagcacctcgagcgactgctcttagttctattcagtcaggaattcagcaagctagatgagaaggtgattaagaggcttggcagttccctgttagactacactgcccagaccaacagggaaatattgtagctacatttgagcctttttgttttaaattactcaaagaatttaaacaagctattaatcagtatggaccaggttctccttttgtaatgggactattaaagaacattgctgtttccagtcagatgattcctactgactgggacgctcttactcaagcttgtctaactcctgcttagttcttacaatttaaaacttggtgggcagatgaagcttccattcaggcttctcacaacacgcaggaccaacctcaaattaatataactgcagaccaacttttgggggttggcagttgggctggtttagatgcacaaatggtcatgcaggatgatgccatagaacagcttagaggagcgtgcattagagcttggggaaaaaaaatcacttcaagtggagaacaataccctttctttagtgctataaaacagggaccagaagaatcatatgtggattttatagctcagttacaggagtctcttaaaaagatgactgcagatttggctgctcaggatatagtgttgcaattattagctttcaacaatgctaatcctgattgccaggctgctctgtgacctatcagagggaaagcacatttagttgattatatcaaggcctgtggtggtatcagaggtaatctgcatcaggccacctgctagcacgggcaatggcaggactgagagtggatacagaaagtactccatttcctggagcttgttttaactgtgggaagcatggtcatactg

SEQ ID NO: 7 - protein, 316 aa, Homo sapiens
MASADELTFHEFEEATNLLADTPDAATTSRSDQLTPQGHVAVAVGSGGSYGAEDEVEEESDKAALLQEQQQQQQPGFWTFSYYQSFFDVDTSQVLDRIKGSLLPRPGHNFVRHHLRNRPDLYGPFWICATLAFVLAVTGNLTLVLAQRRDPSIHYSPQFHKVTVAGISIYCYAWLVPLALWGFLRWRKGVQERMGPYTFLETVCIYGYSLFVFIPMVVLWLIPVPWLQWLFGALALGLSAAGLVFTLWPVVREDTRLVATVLLSVVVLLHALLAMGCKLYFFQSLPPENVAPPPQITSLPSNIALSPTLPQSLAPS

SEQ ID NO: 8 - protein, 316 aa, Homo sapiens; identical to SEQ ID NO: 7

SEQ ID NO: 9 - DNA, 1210 bp, Homo sapiens
aggtgagggggggcggagcgcacctgtggggacgggacgacgagttcaagcctccgtgggtgcagttggtcgccagcgagggatgcggagacgcccctgaacgaccatggcatcggccgacgagctgaccttccatgaattcgaggaggccactaatcttctggctgacaccccagatgcagccaccaccagcagaagcgatcagctgaccccacaagggcacgtggctgtggccgtgggctcaggtggcagctatggagccgaggatgaggtggaggaggagagtgacaaggccgcgctcctgcaggagcagcagcagcagcagcagccgggattctggaccttcagctactatcagagcttctttgacgtggacacctcacaggtcctggaccggatcaaaggctcactgctgccccggcctggccacaactttgtgcggcaccatctgcggaatcggccggatctgtatggccccttctggatctgtgccacgttggcctttgtcctggccgtcactggcaacctgacgctggtgctggcccagaggagggacccctccatccactacagcccccagttccacaaggtgaccgtggcaggcatcagcatctactgctatgcgtggctggtgcccctggccctgtggggcttcctgcggtggcgcaagggtgtccaggagcgcatggggccctacaccttcctggagactgtgtgcatctacggctactccctctttgtcttcatccccatggtggtcctgtggctcatccctgtgccttggctgcagtggctctttggggcgctggccctgggcctgtcagccgccgggctggtattcaccctctggcccgtggtccgtgaggacaccaggctggtggccacagtgctgctgtccgtggtcgtgctgctccacgccctcctggccatgggctgtaagttgtacttcttccagtcgctgcctccggagaacgtggctcctccaccccaaatcacatctctgccctcaaacatcgcgctgtcccctaccttgccgcagtccctggccccctcctaggaaggcccgggtcccacaggcaacacctaagtggaccaacccctctgcctgtcctgccccccagacgatgactgaaggctcctttgacaccttgagatgattctgctactttccagacttttcttacaaagcaaacacttttattttctatgc

SEQ ID NO: 10 - DNA, 1502 bp, Homo sapiens
ggatgttgctgtcaggtctgagtcggttggagtctgacgggtaggcgagacgcgcaggcgcagagagccccagccacgccggcccaggtggcctcagcgagggatgcggagacgcccctgaacgaccatggcatcggccgacgagctgaccttccatgaattcgaggaggccactaatcttctggctgacaccccagatgcagccaccaccagcagaagcgatcagctgaccccacaagggcacgtggctgtggccgtgggctcaggtggcagctatggagccgaggatgaggtggaggaggagagtgacaaggccgcgctcctgcaggagcagcagcagcagcagcagccgggattctggaccttcagctactatcagagcttctttgacgtggacacctcacaggtcctggaccggatcaaaggctcactgctgccccggcctggccacaactttgtgcggcaccatctgcggaatcggccggatctgtatggccccttctggatctgtgccacgttggcctttgtcctggccgtcactggcaacctgacgctggtgctggcccagaggagggacccctccatccactacagcccccagttccacaaggtgaccgtggcaggcatcagcatctactgctatgcgtggctggtgcccctggccctgtggggcttcctgcggtggcgcaagggtgtccaggagcgcatggggccctacaccttcctggagactgtgtgcatctacggctactccctctttgtcttcatccccatggtggtcctgtggctcatccctgtgccttggctgcagtggctctttggggcgctggccctgggcctgtcagccgccgggctggtattcaccctctggcccgtggtccgtgaggacaccaggctggtggccacagtgctgctgtccgtggtcgtgctgctccacgccctcctggccatgggctgtaagttgtacttcttccagtcgctgcctccggagaacgtggctcctccaccccaaatcacatctctgccctcaaacatcgcgctgtcccctaccttgccgcagtccctggccccctcctaggaaggcccgggtcccacaggcaacacctaagtggaccaacccctctgcctgtcctgccccccagacgatgactgaaggctcctttgacaccttgagatgattctgctactttccagacttttcttacaaagcaaacacttttattttctatgcaaaggtgattcagagaatttatataaaggcgggcgaggggcagccgagcagggagctttgggacagggctggggcccccatatcccccccgggccacctgctttccctcctatggctcccctggaacaggagggagagccaagggggcggcccagcctggacagcgcccgctcctgcctgggtgcacacacggcgggcctgagctccagcatctgagtttgggggtatgagaaacaggggagcagaaggagaagaaaactgcctgtgctgc

SEQ ID NO: 11 - protein, 157 aa, Homo sapiens
MLPEQGPQPSTMPLWCLLAACTSLPRQAATMLEEAASPNEAVHASTSGSGALTDQTFTDLSAAEASSEEVPDFMEVPHSVHHKINCFFYLEKQLCQLPSPLCLSSLLTLKLKTTVPAPGRWWSFQPHKAFPLLVGTPGSWQSTIDPAWAAPSQPSPG

SEQ ID NO: 12 - DNA, 474 bp, Homo sapiens
atgcttccagagcaagggccccagccttccacgatgccgctctggtgcctcctcgccgcctgcaccagcctcccaaggcaggcagccaccatgctggaggaagctgcttctcccaacgaggctgtccacgcatcaacatcaggcagtggcgcactcactgatcagacatttacagacctctcagctgccgaggcctcctcagaggaggttcctgacttcatggaggtgccacactctgttcaccataaaattaactgctttttctacttagaaaaacaactctgccaactgccgtccccactgtgtctaagcagcttgcttactttaaaattaaaaacaacggtcccagctcctggcaggtggtggagcttccagcctcacaaggcattcccacttctggtgggcactcctggaagctggcagagcacaatcgatcccgcgtgggcggccccctctcagccaagcccagggtga

SEQ ID NO: 13 - protein, 241 aa, Homo sapiens
MKFILLWALLNLTVALAFNPDYTVSSTPPYLVYLKSDYLPCAGVLIHPLWVITAAHCNLPKLRVILGVTIPADSNEKHLQVIGYEKMIHHPHFSVTSIDHDIMLIKLKTEAELNDYVKLANLPYQTISENTMCSVSTWSYNVCDIYKEPDSLQTVNISVISKPQCRDAYKTYNITENMLCVGIVPGRRQPCKEVSAAPAICNGMLQGILSFADGCVLRADVGIYAKIFYYIPWIENVIQNN

SEQ ID NO: 14 - DNA, 912 bp, Homo sapiens
aaggctggcaaaaaggagaccagacaggaggcgtctgtagagatatcatgaacttcaacttagctttgttttccagagactggagctaaactgggctttcaacatcatcatgaagtttatcctcctctgggccctcttgaatctgactgttgctttggcctttaatccagattacacagtcagctccactcccccttacttggtctatttgaaatctgactacttgccctgcgctggagtcctgatccacccgctttgggtgatcacagctgcacactgcaatttaccaaagcttcgggtgatattgggggttacaatcccagcagactctaatgaaaagcatctgcaagtgattggctatgagaagatgattcatcatccacacttctcagtcacttctattgatcatgacatcatgctaatcaagctgaaaacagaggctgaactcaatgactatgtgaaattagccaacctgccctaccaaactatctctgaaaataccatgtgctctgtctctacctggagctacaatgtgtgtgatatctacaaagagcccgattcactgcaaactgtgaacatctctgtaatctccaagcctcagtgtcgcgatgcctataaaacctacaacatcacggaaaatatgctgtgtgtgggcattgtgccaggaaggaggcagccctgcaaggaagtttctgctgccccggcaatctgcaatgggatgcttcaaggaatcctgtcttttgcggatggatgtgttttgagagccgatgttggcatctatgccaaaattttttactatataccctggattgaaaatgtaatccaaaataactgagctgtggcagttgtggaccatatgacacagcttgtccccatcgttcacctttagaattaaatataaattaactcctc

SEQ ID NO: 15 - protein, 279 aa, Homo sapiens
MMLMQALVLFTLDSLDMLPAVKATWLYGIQITSLLLVCILQFFNSMILGSLLISFNLSVFIARKLQKNLKTGSFLNRLGKLLLHLFMVLCLTLFLNNIIKKILNLKSDEHIFKFLKAKFGLGATRDFDANLYLCEEAFGLLPFNTFGRLSDTLLFYAYIFVLSITVIVAFVVAFHNLSDSTNQQSVGKMEKGTVDLKPETAYNLIHTILFGFLALSTMRMKYLWTSHMCVFASFGLCSPEIWELLLKSVHLYNPKRICIMRYSVPILILLYLCYKNQKS

SEQ ID NO: 16 - protein, 102 aa, Homo sapiens
MMSIRQRREIRATEVSEDFPAQEENVKLENKLPSGCTSRRLWKILSLTIGGTIALCIGLLTSVYLATLHENDLWFSNIKVWSFFDHCIIHSVGSPVVSHVDE

SEQ ID NO: 17 - protein, 716 aa, Homo sapiens
MMSIRQRREIRATEVSEDFPAQEENVKLENKLPSGCTSRRLWKILSLTIGGTIALCIGLLTSVYLATLHENDLWFSNIKEVEREISFRTECGLYYSYYKQMLQAPTLVQGFHGLIYDNKTESMKTINLLQRMNIYQEVFLSILYRVLPIQKYLEPVYFYIYTLFGLQAIYVTALYITSWLLSGTWLSGLLAAFWYVTNRIDTTRVEFTIPLRENWALPFFAIQIAAITYFLRPNLQPLSERLTLLAIFISTFLFSLTWQFNQFMMLMQALVLFTLDSLDMLPAVKATWLYGIQITSLLLVCILQFFNSMILGSLLISFNLSVFIARKLQKNLKTGSFLNRLGKLLLHLFMVLCLTLFLNNIIKKILNLKSDEHIFKFLKAKFGLGATRDFDANLYLCEEAFGLLPFNTFGRLSDTLLFYAYIFVLSITVIVAFVVAFHNLSDSTNQQSVGKMEKGTVDLKPETAYNLIHTILFGFLALSTMRMKYLWTSHMCVFASFGLCSPEIWELLLKSVHLYNPKRICIMRYSVPILILLYLCYKFWPGMMDELSELREFYDPDTVELMNWINSNTPRKAVFAGSMQLLAGVKLCTGRTLTNHPHYEDSSLRERTRAVYQIYAKRAPEEVHALLRSFGTDYVILEDSICYERRHRRGCRLRDLLDIANGHMMDGPGENDPDLKPADHPRFCEEIKRNLPPYVAYFTRVFQNKTFHVYKLSRNK

SEQ ID NO: 18 - protein, 112 aa, Homo sapiens
MMSIRQRREIRATEVSEDFPAQEENVKLENKLPSGCTSRRLWKILSLTIGGTPFALDFLHLSTLPRYMKMIYGFLILRKWSEKSHSEQSVACITPTTSRCCRLQPSCKVITT

SEQ ID NO: 19 - DNA, 1841 bp, Homo sapiens
aagtttgcggagcggcttctgctcgtcggccgtgcggcgaggcagggcctgggctgcgaccccggcggccgctcgcggtcttgggagagctggggcgcgtgcctgaacttcccggctgcccctgtccttggagacctacctgatggggacgccaggtgtgcaggggcgtggcgcgtaggagtgatttggagaacaatgcatgtaagtctgacatcatgatgtccatccggcaaagaagagaaataagagccacagaagtttctgaagactttccagcccaagaagaaaatgtgaagttggaaaataaattgccatctggttgtaccagtagaagattatggaagattttgtcattgacaattggtggaaccattgccctttgcattggacttcttacatctgtctaccttgccacgttacatgaaaatgatttatggttttctaatattaaggaagtggagcgagaaatctcattcagaacagagtgtggcctgtattactcctactacaagcagatgctgcaggctccaaccctcgtgcaaggttttcatggcctaatatatgataataaaactgaatctatgaagacaattaacctccttcagcgaatgaatatttaccaagaggtttttctcagtattttatatagagttctacccatacagaaatatttagagccagtttatttttatatttacaccttatttgggctccaggcgatctatgtcacagctctctacataaccagctggctactcagtggtacatggctgtcaggactgttggcagctttctggtatgtcacaaatagaatagataccacaagagttgagtttaccatcccactgagggagaactgggcgctgccattctttgcaattcagatagcagcaattacatatttcctgagaccaaacttacagcctctttctgaaaggctgacacttcttgccattttcatatcaacttttctctttagtctgacatggcaatttaatcaatttatgatgctgatgcaagcattagtgctgttcacactggactccctggacatgctgccagcagtgaaggcgacatggctgtatggaatacagataacaagtttactcctggtctgcattcttcagttttttaattccatgattcttggatcactgcttatcagttttaacctttcagtattcattgcaagaaaacttcagaaaaatctgaaaactggaagcttccttaataggcttgggaaacttttgttacatttatttatggttttatgtttgacactttttctcaacaacataattaagaaaattcttaacctgaagtcagatgaacacatatttaaatttctgaaggcaaaatttgggcttggagcaacaagggattttgatgcaaatctctatctgtgtgaagaagcttttggcctcctgccttttaatacatttggaaggctttcagatactctgcttttttatgcttacatattcgttctgtccatcacagtgattgtagcattcgttgttgcctttcataatctcagtgattctacaaatcaacaatccgtgggtaaaatggaaaaaggcacagttgacctgaaaccagaaactgcctacaacttaatacataccattctgtttggattcttggcattgagtacaatgagaatgaagtacctctggacgtcacacatgtgtgtgttcgcatcattcggcctatgtagccctgaaatatgggagttacttctgaagtcagtccatctttataacccaaagaggatatgtataatgcgatattcagtaccgatattaatactgctgtatctatgctataagaatcagaaatct

SEQ ID NO: 20 - DNA, 2206 bp, Homo sapiens
cggttctgccctccttgtacccgcggcgcgctgcggcccgtggcgcggccccgttcccgcctagccccgtcggcctccttcccctcccggagccgcgcgtgaggacggctgaggccgcaggagtgatttggagaacaatgcatgtaagtctgacatcatgatgtccatccggcaaagaagagaaataagagccacagaagtttctgaagactttccagcccaagaagaaaatgtgaagttggaaaataaattgccatctggttgtaccagtagaagattatggaagattttgtcattgacaattggtggaaccattgccctttgcattggacttcttacatctgtctaccttgccacgttacatgaaaatgatttatggttttctaatattaaggtatggagtttctttgaccattgtatcattcactcagtgggatctccagtagtaagccatgtggatgaatgaccaaggcaacacagttttgccataaagaatccaatctctagaaaggttggactatagagtgaaataacttttgtgtttattattttaaaataacatattagaatctttttttaaatttttctttattatttatttatttttgagatggagtctcactctgtcacccaggctggagtgcggtggcgcaatcttggctcactacaacctctgcctcgcaggttcaggtgattcttctggcttagcctcccaagtagctgggactataggtgcgtgccaccacacccagctaatttttgtatttttactagagacggggtttcagcatattgaccaggctgatctcgaactcctgaccttgtgatctgcctgtctcagcctcccaaagtgctgggattacaggcgtgagccactgcgtccagccagaatctttatttttcattttaattttttgagatagggtattgctctgtcacccaggctagaatgcagtggtgcaaacatgggtcactgcagcctcaacctcctgggctcaagtgagtatcctgcctaagcttcctgtgtcactgggaccccaggcatgcaccacctcaccaagctaaatttgatttttttgtagagacagggtctcactttgttgcccatgctggtctcgaactcctgggctcaagcgatcctactgccctggtcttccaaaatatgagaatgagccatagcacccagcccagaatttttataatcaagtgagttttttctttttcattaacttattccatttatttagcagttattctaaattagtatttttcaagttatagattgtgaaattagtgcagtaggtcatgagtaacatttttcttaatgaaatcaaaaagaaagaatactatcacatctagtagggttgaggattgttttgtgaaacttttaattttatatatatatatatatgcacaaactgggtcacagtatacaaggtacttccttttcttttttttcttgttggctacaacaggaaaaaaaaaaaacagaaaaggaaataaaaaagccactgctttaaatcatggggtctaaatgtggctccacagagggtcctcagcatgttcatgactatctaatactctgtgcaagtggttttgcagggcatagggcgatggggaagccatatgtttccagggaaaggaactgtaattttaatcagattttcaggagggttagccgggcgtcacgcctgtaatcccagcactttgggaggtcgaggcgggcagatcacttgaagtcaggagttcaagaccagcctggccaacatggtggaaccctatctctactaaaaatacaaaaattagccgggcatggtgacacacacctgtaatctcagctactcaggaggctgaggcacaagaatcacttgaactcgggaggaagaggttgcagtgagctgagatcccaccactgcactccagcctgggcaacagagcaatactctttatcaaaaaaaaaaagaaaaaagttgagggggtggtctgtgactctttaaacacgtttccttgttttctttctctctctctttttcaacatttctagaactcctcttggcattgttttcagaactcgtatataacttacatgtggaaatttgcatccaaatataccttacattttaatctaatatgtcatgatctttaacctaaactgtggtgtctaatgactagttgcttgtaaaaataaacaaacaccttcaaagcc

SEQ ID NO: 21 - DNA, 4456 bp, Homo sapiens
aagtttgcggagcggcttctgctcgtcggccgtgcggcgaggcagggcctgggctgcgaccccggcggccgctcgcggtcttgggagagctggggcgcgtgcctgaacttcccggctgcccctgtccttggagacctacctgatggggacgccaggtgtgcaggggcgtggcgcgtaggagtgatttggagaacaatgcatgtaagtctgacatcatgatgtccatccggcaaagaagagaaataagagccacagaagtttctgaagactttccagcccaagaagaaaatgtgaagttggaaaataaattgccatctggttgtaccagtagaagattatggaagattttgtcattgacaattggtggaaccattgccctttgcattggacttcttacatctgtctaccttgccacgttacatgaaaatgatttatggttttctaatattaaggaagtggagcgagaaatctcattcagaacagagtgtggcctgtattactcctactacaagcagatgctgcaggctccaaccctcgtgcaaggttttcatggcctaatatatgataataaaactgaatctatgaagacaattaacctccttcagcgaatgaatatttaccaagaggtttttctcagtattttatatagagttctacccatacagaaatatttagagccagtttatttttatatttacaccttatttgggctccaggcgatctatgtcacagctctctacataaccagctggctactcagtggtacatggctgtcaggactgttggcagctttctggtatgtcacaaatagaatagataccacaagagttgagtttaccatcccactgagggagaactgggcgctgccattctttgcaattcagatagcagcaattacatatttcctgagaccaaacttacagcctctttctgaaaggctgacacttcttgccattttcatatcaacttttctctttagtctgacatggcaatttaatcaatttatgatgctgatgcaagcattagtgctgttcacactggactccctggacatgctgccagcagtgaaggcgacatggctgtatggaatacagataacaagtttactcctggtctgcattcttcagttttttaattccatgattcttggatcactgcttatcagttttaacctttcagtattcattgcaagaaaacttcagaaaaatctgaaaactggaagcttccttaataggcttgggaaacttttgttacatttatttatggttttatgtttgacactttttctcaacaacataattaagaaaattcttaacctgaagtcagatgaacacatatttaaatttctgaaggcaaaatttgggcttggagcaacaagggattttgatgcaaatctctatctgtgtgaagaagcttttggcctcctgccttttaatacatttggaaggctttcagatactctgcttttttatgcttacatattcgttctgtccatcacagtgattgtagcattcgttgttgcctttcataatctcagtgattctacaaatcaacaatccgtgggtaaaatggaaaaaggcacagttgacctgaaaccagaaactgcctacaacttaatacataccattctgtttggattcttggcattgagtacaatgagaatgaagtacctctggacgtcacacatgtgtgtgttcgcatcattcggcctatgtagccctgaaatatgggagttacttctgaagtcagtccatctttataacccaaagaggatatgtataatgcgatattcagtaccgatattaatactgctgtatctatgctataagttctggccaggaatgatggatgaactctccgagttgagagaattctatgatccagatacagtggagctgatgaactggattaactctaacactccaagaaaggctgtgtttgcgggaagcatgcagttgctggccggagtcaagctgtgcacgggaaggaccctaaccaaccacccgcactatgaagacagcagcctgagagagcggaccagagcggtttatcagatatatgccaagagggcaccagaggaagtgcatgccctcctaaggtccttcggcactgactacgtaatcctggaagacagcatctgctacgagcggaggcaccgccggggctgccgactccgggacctgctggacattgccaacggccacatgatggatggcccaggagagaatgatcctgatttgaaacctgcagaccaccctcgcttctgtgaagagatcaaaagaaacctgcctccctacgtggcctacttcaccagagtgttccagaacaaaaccttccacgtttacaagctgtccagaaacaagtagcgcagatttctgcccagtgtctatttttgatacggagaaactgcatcatgatgaaactcaatagatgacgtttcctatgtaagtaggtagcccaaaccttcaagctgtgatatgagtaagttctacagatgtttacacaagtgttgccatctttgaaagcatcttctacaagcagaagtctttttcgttgtgtgtctatctttctcattaatgttctttagcctaaatgttaacaactttctaagagtgacctagaattatgttgttggagagaatgatgtgtgttccatggatacctggataggcacataacatgttggaagatgagcacctgctcaggatttgaaatacgtttaattttcaggtgacttaagacagctatgattgaatcaactagagatgatgatcgacttatttaatatgatttcactggtgaagaccaattggtagctttttaaaaagcactttagtgtcctgttttaccttaaaatgttataatattttccagttgtcatgctgtcaacattaacaaaaaaaatcatgttaaggctttgtatcaaacattttgttacactctgtctgaaatgtaatgtggagtacttcagcagtatgtgtcatgtattgtgtgtgtctgtgtgtgtgcatgtgcacacatgtgttttaatgctgggcacagaaaagtgttacaagttccatatcgtaagtccttaaaggggcagaaatatatgtagccaagtagaatttattacattttagtgttattattttaaaacttactgatactctttaacctctcctgcagtaatagttttgctttatttcttactcatttcaatttattgggtttgcaaaattttgtaaactttttgtgtttttagcctttgtattttttacagcctagaatcttgcaaagtctgaatattttttaaatgttctatcttaactagttcactaatacagtatttttagcagacagcattttcagacagcattttcataccaagtttgacttgtggtctccaatcttactgggaaggccctggtagtgtaattcttttccttattaaaaggtaaccaagtgcctctaagtcatgcttatttgtaaacaacaaagaagagtatatgtacctgctcaaaatttttttgataatcgcttatataattaatttctaatgatgaggacatgtaaaagttgccagtaagaacatagtatgcatttaattaaatcaagatggctaatggaattaactttctcccctgttcttgccaggtggaaatgatttaagcatttctccttgcagttgtattgaagtaaattaccataggcatcaagatggctgcatcacattttcaaatgattttatattcagttgctacttataaagcagcattcaaaaagtcttttacactgtcatgttggacacaagcagactcagcttttatcaaaacttgtttaaataaaaaattgacagtagctgggttattaaattatgcaactgaaactcctgaattatatcttttctgtatcccttaataagattggagaccactgccgtttaggataatacaataataaaacgttttaatcagtactaaaactttaattaagccaataatgatgcatgcctgttgtagctgacagcatgggtcagtacatccttcagcgagtgccttactctaattgaaaccaagcacacgtaaggtacaatatgttagactctgtgattttgttttcaaaatcctctgttatggctatatttaaatttattttaaatattcctgtatgtattcatctaagcatttgggcatttggagtcttaatatacaagaaacacgtacttaaatttttatgcttatcaccgcaatgatggcaaacagtgattttttttttcatagtttaggtgtcattgttgccagcacctttagtgctcagtcttcagtgaaaaatataaagtgccaaaaaaatcttgcaagacagaatccatacttaacactctttccaagacactgtgaccatgtacagtagctatttcctgatgaccaaatctctcaacgaatcatgttattaataaatatttttagcactcatc

SEQ ID NO: 22 - DNA, 336 bp, Homo sapiens
atgatgtccatccggcaaagaagagaaataagagccacagaagtttctgaagactttccagcccaagaagaaaatgtgaagttggaaaataaattgccatctggttgtaccagtagaagattatggaagattttgtcattgacaattggtggaaccccctttgcattggacttcttacatctgtctaccttgccacgttacatgaaaatgatttatggttttctaatattaaggaagtggagcgagaaatctcattcagaacagagtgtggcctgtattactcctactacaagcagatgctgcaggctccaaccctcgtgcaaggtaattacaact

SEQ ID NO: 23 - protein, 831 aa, Homo sapiens
MKVHMHTKFCLICLLTFIFHHCNHCHEEHDHGPEALHRQHRGMTELEPSKFSKQAAENEKKYYIEKLFERYGENGRLSFFGLEKLLTNLGLGERKVVEINHEDLGHDHVSHLDILAVQEGKHFHSHNHQHSHNHLNSENQTVTSVSTKRNHKCDPEKETVEVSVKSDDKHMHDHNHRLRHHHRLHHHLDHNNTHHFHNDSITPSERGEPSNEPSTETNKTQEQSDVKLPKGKRKKKGRKSNENSEVITPGFPPNHDQGEQYEHNRVHKPDRVHNPGHSHVHLPERNGHDPGRGHQDLDPDNEGELRHTRKREAPHVKNNAIISLRKDLNEDDHHHECLNVTQLLKYYGHGANSPISTDLFTYLCPALLYQIDSRLCIEHFDKLLVEDINKDKNLVPEDEANIGASAWICGIISITVISLLSLLGVILVPIINQGCFKFLLTFLVALAVGTMSGDALLHLLPHSQGGHDHSHQHAHGHGHSHGHESNKFLEEYDAVLKGLVALGGIYLLFIIEHCIRMFKHYKQQRGKQKWFMKQNTEESTIGRKLSDHKLNNTPDSDWLQLKPLAGTDDSVVSEDRLNETELTDLEGQQESPPKNYLCIEEEKIIDHSHSDGLHTIHEHDLHAAAHNHHGENKTVLRKHNHQWHHKHSHHSHGPCHSGSDLKETGIANIAWMVIMGDGIHNFSDGLAIGAAFSAGLTGGISTSIAVFCHELPHELGDFAVLLKAGMTVKQAIVYNLLSAMMAYIGMLIGTAVGQYANNITLWIFAVTAGMFLYVALVDMLPEMLHGDGDNEEHGFCPVGQFILQNLGLLFGFAIMLVIALYEDKIVFDIQF

SEQ ID NO: 24 - protein, 831 aa, Homo sapiens; identical to SEQ ID NO: 23

SEQ ID NO: 25 - DNA, 5227 bp, Homo sapiens
cacgatttggtgcagccggggtttggtaccgagcggagaggagatgcacacggcactcgagtgtgaggaaaaatagaaatgaaggtacatatgcacacaaaattttgcctcatttgtttgctgacatttatttttcatcattgcaaccattgccatgaagaacatgaccatggccctgaagcgcttcacagacagcatcgtggaatgacagaattggagccaagcaaattttcaaagcaagctgctgaaaatgaaaaaaaatactatattgaaaaactttttgagcgttatggtgaaaatggaagattatccttttttggtttggagaaacttttaacaaacttgggccttggagagagaaaagtagttgagattaatcatgaggatcttggccacgatcatgtttctcatttagatattttggcagttcaagagggaaagcattttcactcacataaccaccagcattcccataatcatttaaattcagaaaatcaaactgtgaccagtgtatccacaaaaagaaaccataaatgtgatccagagaaagagacagttgaagtgtctgtaaaatctgatgataaacatatgcatgaccataatcaccgcctacgtcatcaccatcgtttgcatcatcatcttgatcataacaacactcaccattttcataatgattccattactcccagtgagcgtggggagcctagcaatgaaccttcaacagagaccaataaaacccaggaacaatctgatgttaaactaccgaaaggaaagaggaagaaaaaagggaggaaaagtaatgaaaattctgaggttattacaccaggttttccccctaaccatgatcagggtgaacagtatgagcataatcgggtccacaaacctgatcgtgtacataacccaggtcattctcatgtacatcttccagaacgtaatggtcatgatcctggtcgtggacaccaagatcttgatcctgataatgaaggtgaacttcgacatactagaaagagagaagcaccacatgttaaaaataatgcaataatttctttgagaaaagatctaaatgaagatgaccatcatcatgaatgtttgaacgtcactcagttattaaaatactatggtcatggtgccaactctcccatctcaactgatttatttacatacctttgccctgcattgttatatcaaatcgacagcagactttgtattgagcattttgacaaacttttagttgaagatataaataaggataaaaacctggttcctgaagatgaggcaaatataggggcatcagcctggatttgtggtatcatttctatcactgtcattagcctgctttccttgctaggcgtgatcttggttcctatcattaaccaaggatgcttcaaattccttcttacattccttgttgcattagctgtaggaacaatgagtggagacgcccttcttcatctactgccccattctcagggtggacatgatcacagtcaccaacatgcacatgggcatggacattctcatggacatgaatctaacaagtttttggaagaatatgatgctgtattgaaaggacttgttgctctaggaggcatttacttgctatttatcattgaacactgcattagaatgtttaagcactacaaacaacaaagaggaaaacagaaatggtttatgaaacagaacacagaagaatcaactattggaagaaagctttcagatcacaagttaaacaatacaccagattctgactggcttcaactcaagcctcttgccggaactgatgactcggttgtttctgaagatcgacttaatgaaactgaactgacagatttagaaggccaacaagaatcccctcctaaaaattacctttgtatagaagaggagaaaatcatagaccattctcacagtgatggattacataccattcatgagcatgatctccatgctgctgcacataaccaccacggcgagaacaaaactgtgctgaggaagcataatcaccagtggcaccacaagcattctcatcattcccatggcccctgtcattctggatccgatctgaaagaaacaggaatagctaatatagcctggatggtgatcatgggggatggcatccacaacttcagtgatgggctcgcaattggtgcagctttcagtgctggattgacaggaggaatcagtacttctatagccgtcttctgtcatgaactgccacatgaattaggagattttgcagttcttcttaaagcaggcatgactgtaaagcaagcaattgtatacaacctcctctctgccatgatggcttacataggcatgctcataggcacagctgttggtcagtatgccaataacatcacactttggatctttgcagtcactgcaggcatgttcctctatgtagccttggtggatatgcttccagaaatgttgcatggtgatggtgacaatgaagaacatggcttttgtcctgtggggcaattcatccttcagaatttaggattgctctttggatttgccattatgctggtgattgccctctatgaagataaaattgtgtttgacatccagttttgacctttcccagtaatcactgttgattacgagaatgttaccatgcagctttgcatctgttccttgtactgtatgcacattgctcaaaggaaagtcagtggcttgcactacttacaagtttcatagatttgagcctaaccacaagaggctggtgcttagtactgttttccctgcacgtaggggtcttttaaaaatataaagcttgtgataaagagaggagaatatgggactccatgaaccagtgttgatatgtttgattaagacttttcacaaaataatcatataaaacactagtctctttattagtagaaacttctgtggctatgcagaaatagagatcgaaccaaaaaaaatcatttaaactttaaaaatattttaaatggactttggggagacattttttgtgtgttttaagaatgaattgtagtgctctttaattcagctacatatattcatgtggtgatagggatcaacttgacacaactttgaaactgcataaagtagacataggaactagaggaaagctcaggctgcattagagtatgaatttagcattgggaaaagcccttattcttgaatctagagttactatttttgtatatatttgcatagtgtttaaacctgcagcctaaactactgaaatttgtgattgtatgtttgtgtgagcttcagtttaatgaaagattcataatggttctttgtattattataatacttggtgttggggtgttctttctgttttgttttttactttaattttgttttgattttttttttttttttttggcgggggtaggtgagggtttggagcatgtggtctttttaaaaaattgtaaccctctagaaaatatcaaagaaatgaaccagacgtggtttaaatagttgattttcctattttaacagtaccaactagttaattgggaaatgtaagttctgaatgttcacattgctttaccagtttggcactggaaccaagagcacatgtcgtggctggctacaaggttgtaaagcagaaaatcgaagtttaccatgtctgtaatgtgtacatgaagtgtcaatttagaacagttactaggataaactccattattgccatggctgtcatggtacccaagtgacttggaagatgcatttaaattactcagctgaaatcacttgatcatcttgtgccaagatatgctgttggtgcctgatagggattagtcttttaggtgccctgttctcctaccataattgtgaatgatttgtgagaagtgcaagccatgtttatcctgaatttttacttaataatttgtattactagtcatatgcatgtagctttctgtttacatcctatgccacatggtcttcatttatgccaggtaaactgtatttgaactatgtgcagctagctttgttttaatctgcttggcaaccagtgtagctgctgtaacaatctatcttattgttcaaatatataagagccaaactcttttccattccatctaaaatgttttcatttagtactcttctttcctcctactctatgaacttcaaaacaaaaacaaaactttgagagcagcacatgcatccaggtatttatagattattgccagtgtcttttctgtatgctataagcaagggagcttaggtgttatttctttaatttatgcttgaatctgaaaaattatttctgacttactccatggcctccttataataagtagaagttttatatataattaattttcagcattgggcactgaattaggacagtcctcatctcattgcttggcccttcaagcaacctagctaaaaggtgctgatattttatttagtactgccaacttcaagtgatttagatatctatctatctagatttctgaaccaagatatatttatagttcacttttgggtttttatacccacggtaggattctgcattccagcattaaatctgcttcattttagaacctttataaaagcaatagctggaatatactcccagttttaaaataaatgcctgattgatttaaagcaagtaggttatgctgaagtatataaagaagttttatattctctcaaaaatggtattatctttctttatttgctagattcttacaaatcttttaagagggctgtaacagttgctgctagtattagggttccacatcattctaatgtatagtttcaagtcttaatagacaatctgaattccactacatttcttttggctccaacattccttttagcttgaccagtctaatttaaaatgtgtttgttggaggtcattaacgttacttgtacaatgctgtcactgtgtgacatccatatgaattttggtatatatcaatcaatcaatcaatcacattgcattcaatcaatcagctgtgattgattgattatgcttagaaatactatagtaactagatgcagtgtgaattttttccattaacaaacaaacaagtcagtggcttaaatgtgattatggtcctgcaaggtgattcttgctaaaatatctaaacttttgttttgttttaactgaatcattttttaacttaaaaagctggaaaatatcaaatgctgttttttttttttcattgtcaacagtggtgtgtcattttatgtatgttcctaatgcttatggaactcctccaaaataaagttactcaaagag

SEQ ID NO: 26 - DNA, 5432 bp, Homo sapiens
agttgatcactctgaagctttttggctaaagcgtttgggtttagagcttccattactcattcgccttgcccaaggcctcagcaaccgacgttcgaaagccaggagaaaaggcgaatgataaagggcgctccacgcatgcgttaagaagccgccccaactcccccgcggcgttctttcttggaacaaaactagcgcggagccacggaactccgcagtttgcgtagacttgaatttcctattcctcggacgatccatgtggaatccgaaaaatagaaatgaaggtacatatgcacacaaaattttgcctcatttgtttgctgacatttatttttcatcattgcaaccattgccatgaagaacatgaccatggccctgaagcgcttcacagacagcatcgtggaatgacagaattggagccaagcaaattttcaaagcaagctgctgaaaatgaaaaaaaatactatattgaaaaactttttgagcgttatggtgaaaatggaagattatccttttttggtttggagaaacttttaacaaacttgggccttggagagagaaaagtagttgagattaatcatgaggatcttggccacgatcatgtttctcatttagatattttggcagttcaagagggaaagcattttcactcacataaccaccagcattcccataatcatttaaattcagaaaatcaaactgtgaccagtgtatccacaaaaagaaaccataaatgtgatccagagaaagagacagttgaagtgtctgtaaaatctgatgataaacatatgcatgaccataatcaccgcctacgtcatcaccatcgtttgcatcatcatcttgatcataacaacactcaccattttcataatgattccattactcccagtgagcgtggggagcctagcaatgaaccttcaacagagaccaataaaacccaggaacaatctgatgttaaactaccgaaaggaaagaggaagaaaaaagggaggaaaagtaatgaaaattctgaggttattacaccaggttttccccctaaccatgatcagggtgaacagtatgagcataatcgggtccacaaacctgatcgtgtacataacccaggtcattctcatgtacatcttccagaacgtaatggtcatgatcctggtcgtggacaccaagatcttgatcctgataatgaaggtgaacttcgacatactagaaagagagaagcaccacatgttaaaaataatgcaataatttctttgagaaaagatctaaatgaagatgaccatcatcatgaatgtttgaacgtcactcagttattaaaatactatggtcatggtgccaactctcccatctcaactgatttatttacatacctttgccctgcattgttatatcaaatcgacagcagactttgtattgagcattttgacaaacttttagttgaagatataaataaggataaaaacctggttcctgaagatgaggcaaatataggggcatcagcctggatttgtggtatcatttctatcactgtcattagcctgctttccttgctaggcgtgatcttggttcctatcattaaccaaggatgcttcaaattccttcttacattccttgttgcattagctgtaggaacaatgagtggagacgcccttcttcatctactgccccattctcagggtggacatgatcacagtcaccaacatgcacatgggcatggacattctcatggacatgaatctaacaagtttttggaagaatatgatgctgtattgaaaggacttgttgctctaggaggcatttacttgctatttatcattgaacactgcattagaatgtttaagcactacaaacaacaaagaggaaaacagaaatggtttatgaaacagaacacagaagaatcaactattggaagaaagctttcagatcacaagttaaacaatacaccagattctgactggcttcaactcaagcctcttgccggaactgatgactcggttgtttctgaagatcgacttaatgaaactgaactgacagatttagaaggccaacaagaatcccctcctaaaaattacctttgtatagaagaggagaaaatcatagaccattctcacagtgatggattacataccattcatgagcatgatctccatgctgctgcacataaccaccacggcgagaacaaaactgtgctgaggaagcataatcaccagtggcaccacaagcattctcatcattcccatggcccctgtcattctggatccgatctgaaagaaacaggaatagctaatatagcctggatggtgatcatgggggatggcatccacaacttcagtgatgggctcgcaattggtgcagctttcagtgctggattgacaggaggaatcagtacttctatagccgtcttctgtcatgaactgccacatgaattaggagattttgcagttcttcttaaagcaggcatgactgtaaagcaagcaattgtatacaacctcctctctgccatgatggcttacataggcatgctcataggcacagctgttggtcagtatgccaataacatcacactttggatctttgcagtcactgcaggcatgttcctctatgtagccttggtggatatgcttccagaaatgttgcatggtgatggtgacaatgaagaacatggcttttgtcctgtggggcaattcatccttcagaatttaggattgctctttggatttgccattatgctggtgattgccctctatgaagataaaattgtgtttgacatccagttttgacctttcccagtaatcactgttgattacgagaatgttaccatgcagctttgcatctgttccttgtactgtatgcacattgctcaaaggaaagtcagtggcttgcactacttacaagtttcatagatttgagcctaaccacaagaggctggtgcttagtactgttttccctgcacgtaggggtcttttaaaaatataaagcttgtgataaagagaggagaatatgggactccatgaaccagtgttgatatgtttgattaagacttttcacaaaataatcatataaaacactagtctctttattagtagaaacttctgtggctatgcagaaatagagatcgaaccaaaaaaaatcatttaaactttaaaaatattttaaatggactttggggagacattttttgtgtgttttaagaatgaattgtagtgctctttaattcagctacatatattcatgtggtgatagggatcaacttgacacaactttgaaactgcataaagtagacataggaactagaggaaagctcaggctgcattagagtatgaatttagcattgggaaaagcccttattcttgaatctagagttactatttttgtatatatttgcatagtgtttaaacctgcagcctaaactactgaaatttgtgattgtatgtttgtgtgagcttcagtttaatgaaagattcataatggttctttgtattattataatacttggtgttggggtgttctttctgttttgttttttactttaattttgttttgattttttttttttttttttggcgggggtaggtgagggtttggagcatgtggtctttttaaaaaattgtaaccctctagaaaatatcaaagaaatgaaccagacgtggtttaaatagttgattttcctattttaacagtaccaactagttaattgggaaatgtaagttctgaatgttcacattgctttaccagtttggcactggaaccaagagcacatgtcgtggctggctacaaggttgtaaagcagaaaatcgaagtttaccatgtctgtaatgtgtacatgaagtgtcaatttagaacagttactaggataaactccattattgccatggctgtcatggtacccaagtgacttggaagatgcatttaaattactcagctgaaatcacttgatcatcttgtgccaagatatgctgttggtgcctgatagggattagtcttttaggtgccctgttctcctaccataattgtgaatgatttgtgagaagtgcaagccatgtttatcctgaatttttacttaataatttgtattactagtcatatgcatgtagctttctgtttacatcctatgccacatggtcttcatttatgccaggtaaactgtatttgaactatgtgcagctagctttgttttaatctgcttggcaaccagtgtagctgctgtaacaatctatcttattgttcaaatatataagagccaaactcttttccattccatctaaaatgttttcatttagtactcttctttcctcctactctatgaacttcaaaacaaaaacaaaactttgagagcagcacatgcatccaggtatttatagattattgccagtgtcttttctgtatgctataagcaagggagcttaggtgttatttctttaatttatgcttgaatctgaaaaattatttctgacttactccatggcctccttataataagtagaagttttatatataattaattttcagcattgggcactgaattaggacagtcctcatctcattgcttggcccttcaagcaacctagctaaaaggtgctgatattttatttagtactgccaacttcaagtgatttagatatctatctatctagatttctgaaccaagatatatttatagttcacttttgggtttttatacccacggtaggattctgcattccagcattaaatctgcttcattttagaacctttataaaagcaatagctggaatatactcccagttttaaaataaatgcctgattgatttaaagcaagtaggttatgctgaagtatataaagaagttttatattctctcaaaaatggtattatctttctttatttgctagattcttacaaatcttttaagagggctgtaacagttgctgctagtattagggttccacatcattctaatgtatagtttcaagtcttaatagacaatctgaattccactacatttcttttggctccaacattccttttagcttgaccagtctaatttaaaatgtgtttgttggaggtcattaacgttacttgtacaatgctgtcactgtgtgacatccatatgaattttggtatatatcaatcaatcaatcaatcacattgcattcaatcaatcagctgtgattgattgattatgcttagaaatactatagtaactagatgcagtgtgaattttttccattaacaaacaaacaagtcagtggcttaaatgtgattatggtcctgcaaggtgattcttgctaaaatatctaaacttttgttttgttttaactgaatcattttttaacttaaaaagctggaaaatatcaaatgctgttttttttttttcattgtcaacagtggtgtgtcattttatgtatgttcctaatgcttatggaactcctccaaaataaagttactcaaagagagcaaata

SEQ ID NO: 27 - protein, 543 aa, Homo sapiens [sequence omitted]

SEQ ID NO: 28 - protein, 938 aa, Homo sapiens
MPALEHMNQILHILFVFLPFLWALGTLPPPDALLLWAMEQVLEFGLGGSSMSTHLRLLVMFIMSAGTAIASYFIPSTVGVVLFMTGFGFLLSLNLSDMGHKIGTKSKDLPSGPEKHFSWKECLFYIIILVLALLETSLLHHFAGFSQISKSNSQAIVGYGLMILLIILWILREIQSVYIIGIFRNPFYPKDVQTVTVFFEKQTRLMKIGIVRRILLTLVSPFAMIAFLSLDSSLQGLHSVSVCIGFTRAFRMVWQNTENALLETVIVSTVHLISSTDIWWNRSLDTGLRLLLVGIIRDRLIQFISKLQFAVTVLLTSWTEKKQRRKTTATLCILNIVFSPFVLVIIVFSTLLSSPLLPLFTLPVFLVGFPRPIQSWPGAAGTTACVCADTVYYYQMVPRLTAVLQTAMAAGSLGLLLPGSHYLGRFQDRLMWIMILECGYTYCSINIKGLELQETSCHTAEARRVDEVFEDAFEQEYTRVCSLNEHFGNVLTPCTVLPVKLYSDARNVLSGIIDSHENLKEFKGDLIKVLVWILVQYCSKRPGMKENVHNTENKGKAPLMLPALNTLPPPKSPEDIDSLNSETFNDWSDDNIFDDEPTIKKVIEEKHQLKDLPGTNLFIPGSVESQRVGDHSTGTVPENDLYKAVLLGYPAVDKGKQEDMPYIPLMEFSCSHSHLVCLPAEWRTSCMPSSKMKEMSSLFPEDWYQFVLRQLECYHSEEKASNVLEEIAKDKVLKDFYVHTVMTCYFSLFGIDNMAPSPGHILRVYGGVLPWSVALDWLTEKPELFQLALKAFRYTLKLMIDKASLGPIEDFRELIKYLEEYERDWYIGLVSDEKWKEAILQEKPYLFSLGYDSNMGIYTGRVLSLQELLIQVGKLNPEAVRGQWANLSWELLYATNDDEERYSIQAHPLLLRNLTVQAAEPPLGYPIYSSKPLHIHLY

SEQ ID NO: 29 - protein, 230 aa, Homo sapiens
MSPDVPLLNDYKQDFFLKRFPQTVLGGPRFKLGYCAPPYIYVNQIILFLMPWVWGGVGTLLYQLGILKDYYTAALSGGLMLFTAFVIQFTSLYAKNKSTTVERILTTDILAEEDEHEFTSCTGAETVKFLIPGKKYVANTVFHSILAGLACGLGTWYLLPNRITLLYGSTGGTALLFFFGWMTLCIAEYSLIVNTATETATFQTQDTYEIIPLMRPLYIFFFVSVDLAHR

SEQ ID NO: 30 - protein, 1172 aa, Homo sapiens
MSPDVPLLNDYKQDFFLKRFPQTVLGGPRFKLGYCAPPYIYVNQIILFLMPWVWGGVGTLLYQLGILKDYYTAALSGGLMLFTAFVIQFTSLYAKNKSTTVERILTTDILAEEDEHEFTSCTGAETVKFLIPGKKYVANTVFHSILAGLACGLGTWYLLPNRITLLYGSTGGTALLFFFGWMTLCIAEYSLIVNTATETATFQTQDTYEIIPLMRPLYIFFFVSVDLAHRFVVNMPALEHMNQILHILFVFLPFLWALGTLPPPDALLLWAMEQVLEFGLGGSSMSTHLRLLVMFIMSAGTAIASYFIPSTVGVVLFMTGFGFLLSLNLSDMGHKIGTKSKDLPSGPEKHFSWKECLFYIIILVLALLETSLLHHFAGFSQISKSNSQAIVGYGLMILLIILWILREIQSVYIIGIFRNPFYPKDVQTVTVFFEKQTRLMKIGIVRRILLTLVSPFAMIAFLSLDSSLQGLHSVSVCIGFTRAFRMVWQNTENALLETVIVSTVHLISSTDIWWNRSLDTGLRLLLVGIIRDRLIQFISKLQFAVTVLLTSWTEKKQRRKTTATLCILNIVFSPFVLVIIVFSTLLSSPLLPLFTLPVFLVGFPRPIQSWPGAAGTTACVCADTVYYYQMVPRLTAVLQTAMAAGSLGLLLPGSHYLGRFQDRLMWIMILECGYTYCSINIKGLELQETSCHTAEARRVDEVFEDAFEQEYTRVCSLNEHFGNVLTPCTVLPVKLYSDARNVLSGIIDSHENLKEFKGDLIKVLVWILVQYCSKRPGMKENVHNTENKGKAPLMLPALNTLPPPKSPEDIDSLNSETFNDWSDDNIFDDEPTIKKVIEEKHQLKDLPGTNLFIPGSVESQRVGDHSTGTVPENDLYKAVLLGYPAVDKGKQEDMPYIPLMEFSCSHSHLVCLPAEWRTSCMPSSKMKEMSSLFPEDWYQFVLRQLECYHSEEKASNVLEEIAKDKVLKDFYVHTVMTCYFSLFGIDNMAPSPGHILRVYGGVLPWSVALDWLTEKPELFQLALKAFRYTLKLMIDKASLGPIEDFRELIKYLEEYERDWYIGLVSDEKWKEAILQEKPYLFSLGYDSNMGIYTGRVLSLQELLIQVGKLNPEAVRGQWANLSWELLYATNDDEERYSIQAHPLLLRNLTVQAAEPPLGYPIYSSKPLHIHLY

SEQ ID NO: 31 - protein, 873 aa, Homo sapiens
MPALEHMNQILHILFVFLPFLWALGTLPPPDALLLWAMEQVLEFGLGGSSMSTHLRLLVMFIMSAGTAIASYFIPSTVGVVLFMTGFGFLLSLNLSDMGHKIGTKSKDLPSGPEKHFSWKECLFYIIILVLALLETSLLHHFAGFSQISKSNSQAIVGYGLMILLIILWILREIQSVYIIGIFRNPFYPKDVQTVTVFFEKQTRLMKIGIVRRILLTLVSPFAMIAFLSLDSSLQGLHSVSVCIGFTRAFRMVWQNTENALLETVIVSTVHLISSTDIWWNRSLDTGLRLLLVGIIRDRLIQFISKLQFAVTVLLTSWTEKKQRRKTTATLCILNIVFSPFVLVIIVFSTLLSSPLLPLFTLPVFLVGFPRPIQSWPGAAGTTACVCADTVYYYQMVPRLTAVLQTAMAAGSLGLLLPGSHYLGRFQDRLMWIMILECGYTYCSINIKGLELQETSCHTAEARRVDEVFEDAFEQEYTRVCSLNEHFGNVLTPCTVLPVKLYSDARNVLSGIIDSHENLKEFKGDLIKVLVWILVQYCSKRPGMKENVHNTENKGKAPLMLPALNTLPPPKSPEDIDSLNSETFNDWSDDNIFDDEPTIKKVIEEKHQLKDLPGTNLFIPGSVESQRVGDHSTGTVPENDLYKAVLLGYPAVDKGKQEDMPYIPLMEFSCSHSHLVCLPAEWRTSCMPSSKMKEMSSLFPEDWYQFVLRQLECYHSEEKASNVLEEIAKDKVLKDFYVHTVMTCYFSLFGIDNMAPSPGHILRVYGGVLPWSVALDWLTEKPELFQLALKAFRYTLKLMIDKASLGPIEDFRELIKYLEEYERDWYIGLVSDEKWKEAILQEKPYLFSLGYDSNMPGPALEISRVNRNLWSQI

SEQ ID NO: 32 - DNA, 3927 bp, Homo sapiens
aacgacgctcttgcgtaaaggcccggcccaagggaacgttcagggcgtctcggctttccccgctgctgcttctgctaggcccagtgcgagaccagagcacgagcgactcccgtcgtccccggccaggcagatgttggcctagtcctggcgcgaacgaagcgcgctatttccctgcttcctctaggccaagcctgctttacggcagggcccgcctcgggagcgagcacagaccggggcagcgaggccagccaggcgccgacgaggtccccgaacgcgcacgcgctccgttcagctccgggtggcggccgccggagtagacgttagccatggaaaccgagagctggcccgggcggggccgcggtgagctcgttattcggccgccgcagcttttctgcctccgcattcgggcactaaccaacctcccggcgggagcgcccagcccgagtttacctgcaaaaatgcggtccctgggatgccttcgcgtcttctcttccctcgggtgacttgaggtttgtggtaaatatgccagctctagaacacatgaatcagattttacacatcttgtttgtatttttaccctttctgtgggcacttgggactctgcccccacccgatgcacttctcttatgggcaatggagcaggttttagagttcggccttggaggctcatctatgtcaacccacttacggttattagtaatgttcatcatgtctgctggaacagctatagcatcatatttcattccaagcactgttggtgtggttcttttcatgactggatttggtttcttgctgagtctgaacttaagtgatatgggtcacaaaattggaaccaaatctaaggatttacccagtggtccggaaaaacatttttcatggaaggaatgccttttctacatcattatattagtcttggctcttttagaaactagcttgcttcatcactttgctggcttctcacagatttctaaaagcaattcccaggctattgtgggctatggtttgatgatattacttataatactgtggatacttagagaaattcaaagcgtatatatcattggaattttccgaaatcccttttatccgaaggatgtgcaaactgtgactgtattctttgagaagcaaactaggctcatgaagattggtattgtcagacggattttgctaactttagtatcaccttttgccatgatagcatttctttcattggacagttccttacaagggctccactcagtgtctgtctgtattggattcacaagagcctttagaatggtatggcagaatacagaaaatgctttattggagacagtcattgtatcaacagtacacttgatctccagtacagacatatggtggaacagaagcctggatacaggactcagactcttactggttggtatcatacgtgatcgtttgattcagttcatctctaaattgcagtttgccgtgactgtgcttttgacatcatggacagagaaaaaacaacgtcgaaaaacaactgccactttatgtatactcaacattgtcttttctccattcgtgttggtcatcatagttttttctacactactctcttctcccttactccctcttttcacccttcctgtgttcttggtggggtttccccgacctattcagagttggccaggagcagcaggcaccacagcctgtgtgtgtgcagatacagtgtactactaccaaatggtgcccaggttgactgctgtactgcagactgcaatggcagctggaagtttaggtctcctcctacctggatctcattacttgggccgttttcaggatcgtttaatgtggataatgattctggaatgtggctatacttactgctctattaacattaaggggttagaattgcaggaaacatcctgtcatactgcagaagctcgcagagttgatgaagtttttgaagatgcttttgagcaagaatacacaagagtatgttcccttaatgaacactttggaaatgtcttgacaccctgtactgttttgcctgtgaaattgtattctgatgccaggaatgttctatcaggcataattgattctcatgaaaacttaaaagaatttaaaggtgacctcattaaagtacttgtgtggatacttgttcaatactgctccaaaaggcctggcatgaaagagaatgttcacaacactgaaaataaagggaaagcacctctaatgttgcctgctttgaacactttgccacctcccaaatccccagaagacatagacagtttaaattcagaaacttttaatgactggtctgatgataatatttttgatgatgagccaactatcaaaaaagtaatagaagaaaaacatcagttgaaagatttgccaggtacaaatttgtttattccaggatcagtagaatcacagagggttggtgatcattctacaggcactgttcctgaaaacgatctttacaaagcagttctattaggataccctgctgttgacaaaggaaaacaagaggacatgccatatattcctctcatggagttcagttgttcacattctcacttagtatgcttacccgcagagtggaggactagctgtatgcccagttccaaaatgaaggagatgagctcgttatttccagaagactggtaccaatttgttctaaggcagttggaatgttatcattcagaagagaaggcctcaaatgtactggaagaaattgccaaggacaaagttttaaaagacttttatgttcatacagtaatgacttgttattttagtttatttggaatagacaatatggctcctagtcctggtcatatattgagagtttacggtggtgttttgccttggtctgttgctttggactggctcacagaaaagccagaactgtttcaactagcactgaaagcattcaggtatactctgaaactaatgattgataaagcaagtttaggtccaatagaagactttagagaactgattaagtaccttgaagaatatgaacgtgactggtacattggtttggtatctgatgaaaagtggaaggaagcaattttacaagaaaagccatacttgttttctctggggtatgattctaatatgggaatttacactgggagagtgcttagccttcaagaattattgatccaagtgggaaagttaaatcctgaagctgttagaggtcagtgggccaatctttcatgggaattactttatgccacaaacgatgatgaagaacgttatagtatacaagctcatccactacttttaagaaatcttacggtacaagcagcagaacctcccctgggatatccgatttattcttcaaaacctctccacatacatttgtattagagctcattttgactgtaatgtcatcaaatgcaatgtttttattttttcatcctaaaaaagtaactgtgattcttgtaacttgaggacttctccacacccccattcagatgcctgagaacagctaagctccgtaaagttggttctcttagccatcttaatggttctaaaaaacagcaaaaacatctttatgtctaagataaaagaactatttggccaatatttgtgccctctggactttagtaggctttggtaaatgtgagaaaacttttgtagaattatcatataatgaattttgtaatgctttcttaaatgtgttataggtgaattgccatacaaagttaacagctatgtaatttttacatacttaagagataaacatatcagtgttctaagtagtgataatggatcctgttgaaggttaacataatgtgtatatatttgtttgaaatataatttatagtattttcaaatgtgctgatttattttgacatctaatatctgaatgtttttgtatcaagtagtttgttttcatagacttcaattcataaactttaaaaaacttttaataaaatattttccttccttttc

SEQ ID NO: 33 - DNA, 3932 bp, Homo sapiens
aacgacgctcttgcgtaaaggcccggcccaagggaacgttcagggcgtctcggctttccccgctgctgcttctgctaggcccagtgcgagaccagagcacgagcgactcccgtcgtccccggccaggcagatgttggcctagtcctggcgcgaacgaagcgcgctatttccctgcttcctctaggccaagcctgctttacggcagggcccgcctcgggagcgagcacagaccggggcagcgaggccagccaggcgccgacgaggtccccgaacgcgcacgcgctccgttcagctccgggtggcggccgccggagtagacgttagccatggaaaccgagagctggcccgggcggggccgcggtgagctcgttattcggccgccgcagcttttctgcctccgcattcgggcactaaccaacctcccggcgggagcgcccagcccgagtttacctgcaaaaatgcggtccctgggatgccttcgcgtcttctcttccctcgggtgacttgaggtttgtggtaaatatgccagctctagaacacatgaatcagattttacacatcttgtttgtatttttaccctttctgtgggcacttgggactctgcccccacccgatgcacttctcttatgggcaatggagcaggttttagagttcggccttggaggctcatctatgtcaacccacttacggttattagtaatgttcatcatgtctgctggaacagctatagcatcatatttcattccaagcactgttggtgtggttcttttcatgactggatttggtttcttgctgagtctgaacttaagtgatatgggtcacaaaattggaaccaaatctaaggatttacccagtggtccggaaaaacatttttcatggaaggaatgccttttctacatcattatattagtcttggctcttttagaaactagcttgcttcatcactttgctggcttctcacagatttctaaaagcaattcccaggctattgtgggctatggtttgatgatattacttataatactgtggatacttagagaaattcaaagcgtatatatcattggaattttccgaaatcccttttatccgaaggatgtgcaaactgtgactgtattctttgagaagcaaactaggctcatgaagattggtattgtcagacggattttgctaactttagtatcaccttttgccatgatagcatttctttcattggacagttccttacaagggctccactcagtgtctgtctgtattggattcacaagagcctttagaatggtatggcagaatacagaaaatgctttattggagacagtcattgtatcaacagtacacttgatctccagtacagacatatggtggaacagaagcctggatacaggactcagactcttactggttggtatcatacgtgatcgtttgattcagttcatctctaaattgcagtttgccgtgactgtgcttttgacatcatggacagagaaaaaacaacgtcgaaaaacaactgccactttatgtatactcaacattgtcttttctccattcgtgttggtcatcatagttttttctacactactctcttctcccttactccctcttttcacccttcctgtgttcttggtggggtttccccgacctattcagagttggccaggagcagcaggcaccacagcctgtgtgtgtgcagatacagtgtactactaccaaatggtgcccaggttgactgctgtactgcagactgcaatggcagctggaagtttaggtctcctcctacctggatctcattacttgggccgttttcaggatcgtttaatgtggataatgattctggaatgtggctatacttactgctctattaacattaaggggttagaattgcaggaaacatcctgtcatactgcagaagctcgcagagttgatgaagtttttgaagatgcttttgagcaagaatacacaagagtatgttcccttaatgaacactttggaaatgtcttgacaccctgtactgttttgcctgtgaaattgtattctgatgccaggaatgttctatcaggcataattgattctcatgaaaacttaaaagaatttaaaggtgacctcattaaagtacttgtgtggatacttgttcaatactgctccaaaaggcctggcatgaaagagaatgttcacaacactgaaaataaagggaaagcacctctaatgttgcctgctttgaacactttgccacctcccaaatccccagaagacatagacagtttaaattcagaaacttttaatgactggtctgatgataatatttttgatgatgagccaactatcaaaaaagtaatagaagaaaaacatcagttgaaagatttgccaggtacaaatttgtttattccaggatcagtagaatcacagagggttggtgatcattctacaggcactgttcctgaaaacgatctttacaaagcagttctattaggataccctgctgttgacaaaggaaaacaagaggacatgccatatattcctctcatggagttcagttgttcacattctcacttagtatgcttacccgcagagtggaggactagctgtatgcccagttccaaaatgaaggagatgagctcgttatttccagaagactggtaccaatttgttctaaggcagttggaatgttatcattcagaagagaaggcctcaaatgtactggaagaaattgccaaggacaaagttttaaaagacttttatgttcatacagtaatgacttgttattttagtttatttggaatagacaatatggctcctagtcctggtcatatattgagagtttacggtggtgttttgccttggtctgttgctttggactggctcacagaaaagccagaactgtttcaactagcactgaaagcattcaggtatactctgaaactaatgattgataaagcaagtttaggtccaatagaagactttagagaactgattaagtaccttgaagaatatgaacgtgactggtacattggtttggtatctgatgaaaagtggaaggaagcaattttacaagaaaagccatacttgttttctctggggtatgattctaatatgggaatttacactgggagagtgcttagccttcaagaattattgatccaagtgggaaagttaaatcctgaagctgttagaggtcagtgggccaatctttcatgggaattactttatgccacaaacgatgatgaagaacgttatagtatacaagctcatccactacttttaagaaatcttacggtacaagcagcagaacctcccctgggatatccgatttattcttcaaaacctctccacatacatttgtattagagctcattttgactgtaatgtcatcaaatgcaatgtttttattttttcatcctaaaaaagtaactgtgattcttgtaacttgaggacttctccacacccccattcagatgcctgagaacagctaagctccgtaaagttggttctcttagccatcttaatggttctaaaaaacagcaaaaacatctttatgtctaagataaaagaactatttggccaatatttgtgccctctggactttagtaggctttggtaaatgtgagaaaacttttgtagaattatcatataatgaattttgtaatgctttcttaaatgtgttataggtgaattgccatacaaagttaacagctatgtaatttttacatacttaagagataaacatatcagtgttctaagtagtgataatggatcctgttgaaggttaacataatgtgtatatatttgtttgaaatataatttatagtattttcaaatgtgctgatttattttgacatctaatatctgaatgtttttgtatcaagtagtttgttttcatagacttcaattcataaactttaaaaaacttttaataaaatattttccttccttttcaaata

SEQ ID NO: 34 - DNA, 4073 bp, Homo sapiens
tcttgcgtaaaggcccggcccaagggaacgttcagggcgtctcggctttccccgctgctgcttctgctaggcccagtgcgagaccagagcacgagcgactcccgtcgtccccggccaggcagatgttggcctagtcctggcgcgaacgaagcgcgctatttccctgcttcctctaggccaagcctgctttacggcagggcccgcctcgggagcgagcacagaccggggcagcgaggccagccaggcgccgacgaggtccccgaacgcgcacgcgctccgttcagctccgggtggcggccgccggagtagacgttagccatggaaaccgagagctggcccgggcggggccgcggtgagctcgttattcggccgccgcagcttttctgcctccgcattcgggcactaaccaacctcccggcgggagcgcccagcccgagtttacctgcaaaaatgcggtccctgggatgccttcgcgtcttctcttccctcgggtgacttgagaaactgctgtgttacagaaaagcatgtgactttcagaataatcccgagtgaggatgagtccagatgtgcctctactgaatgattacaagcaggacttctttctgaagcgctttccacagactgttcttggaggccctcgattcaaattaggctattgtgcccctccttacatatatgttaatcaaattattctttttctaatgccatgggtttggggtggagtcggaacacttttataccagttaggcatcctgaaagactattatacagcagcactttcaggtggattaatgctttttactgcatttgtcatccagttcacaagtttatacgccaaaaacaaatcaacaacagtagaaagaatactaaccacggatatcttagcagaggaggatgagcatgaatttaccagttgtactggtgctgagactgtcaaatttctcattcctggcaagaaatatgtagccaatacagtttttcattctattcttgctggattagcgtgtggtcttggaacatggtatctgctcccaaatagaataaccttgctgtatggcagtacaggaggcactgctctactattcttctttggatggatgacactatgtatagcagaatattctttaattgtaaacacagctacagagactgcgactttccaaacacaggatacttatgaaattattcctcttatgagacctctttatatttttttctttgtttctgtggatctggcacacaggtaaaaacctaccaaatactttgtaactaactttgtttttaagtatacagagtaagagagctttccttttagtgttacaaaaaaatgaatccatggattaaaaatcatcaaaccattgggtgacaggttattttgataattattcttttaggattaatctctgtaaaacatactaaagcaatagttaaaacttattaaagagtttttttaaaaaaccctttttgagataaggaacttttcaattttgtgtttcactttaaataaggagctttgagtttttaagatagcctggctaaaacctgtgtaaggagatggaactttcctgtggggggaaagaagaaattaaaatttatacatataaatattatatacagattgaatgaatttaagacaaatacaaaatttatttctaattttatgatagcaacaatagtagaagtaatattgattttttaaaaaccaacttgttacagaagaaaagtagaaaatagtttttttaacagacataattgttcacaaaattgtttgataaccccttttacttgccttttcaagtttgacttttctttctccgtctccgtagattgcagctttcttttcttaggttagtgtccaggtagaaatgttcagcatgttatggactgaatatttgtgtctctccagaattcatatattacagtcttaaaccccaatgtgatggtattttgagatgaggcctttgggaggtaattaggtcatgaaggtgggtccttggtatgatgggactagtccccttatgagaagaggtaccagagagcttgatttgtccctctctgtgccatctaaggacacagcaaaaatgtggccacctgcaagtcaaagaagagagctctcatcaaaacctgaccatactggcagcctgatcatggtctttcggccttcagaagtgtgagaaagtaaattgatgttgtttaagccactcagcttatggcatttttttatggcagcccatgctgattaagattttgctaccaagaagtgggatgcctttgtaccaaatacctaaaaatgtggaaatggctttgtaactgttggtaatgggtagaggctgaaagaggtttttttgtttgtttctttgttttgttttgtttgagatggagtcttcactctcttgcccaggctggagtgcagtggcacaatcttggcccactgcaacgtccgcctcctggcttcaagtgatactcctccctgagcctcctgagtagctggaattacagacatgcgccaccatgcccggctaatttttatatttttaatagagacagggtttcgccatgttggccaggctggtcccaaagtcctgacctcaagtgatccacccgcctcggcctcccaaagttctgggattacaggcttgagccatcacacccagccaagggttttgatgtgcatgctagaaatatggacattaagggtgattctgatgaagtctgaagtcccctgaacccagaggaaaggcagtccttgttacaaagcgtcaaagaacttagctgaactgtgtcctagtgttttgtggagccatgaagttggatacctacgtaaggagagttcaaagcagtgttgaaaggagcagttggcttctcctgtttctagtaaaatgtgaaaggagagagatggattgaagaagggattgttaagcaagagaaggaaccagaacttgaagatttggaaaattttcagcctggattatccatattgtaaaacatgagaaagcatattctgaagagaacaccaaaagtgtggggctggactgtccctcagtaaagagcttttggcattatgtgagtagaaacactgccagtttgaattgaagtggttggagacaggaagtaatgaaggccgacagttgaacttcttggatttgacaggatgtaatgatagaactgttcagctacaaaagtgcagtattcttcaagaaaaggggaaaattatgccaaaggtgatttaagggtcttgagggctaccacctgtttcaacaagtcagctagcctctacccaaagcctcgggagcagaactgaacttcagagccacagaagcaggaccctcacctagagcactggcggtgacctgccaccccagtggcctggtgggcagagtatggaaccaaacagaattattctcaagctctaagatctaatggaatttgccttgctaggttttgacttccttgggatcatcaccctttttttcctgtttctcctattggagtggggatgtctcttctatacctgccctacccttagattttggaagcacgtaactcatctggtttcacagattcacagctggagaggaattttgcctcaggatggattgtacctaggtctcatccatatctgatttagatgagactttgaattttagccatcagagttaatgctagaatgagtgaagactttgggggatatggggatggaatgaatgtcttttgcatttgaatttggaaaggacatgagttttgaggggccagggatggaatgttatagactaattgtgttccctcaaaaatgtttatgttgaagccctaacccccaatgtgttggtatttggagatgggcctctgggaggtagtttatgaaggtgagaccctagtctgataggattagtgcccttaggagagatgccaaagagcttgatgtctctctttttgctacaaaaagacacagcaaaaaggcagccatgtgtaagccaggaagagagtcttcaccagaacctgactatactggcagcctgatcttgtacttgtagcccccagaactgttagaaaataaatttctgttgtttaagcca

SEQ ID NO: 35 - DNA, 4666 bp, Homo sapiens
tcttgcgtaaaggcccggcccaagggaacgttcagggcgtctcggctttccccgctgctgcttctgctaggcccagtgcgagaccagagcacgagcgactcccgtcgtccccggccaggcagatgttggcctagtcctggcgcgaacgaagcgcgctatttccctgcttcctctaggccaagcctgctttacggcagggcccgcctcgggagcgagcacagaccggggcagcgaggccagccaggcgccgacgaggtccccgaacgcgcacgcgctccgttcagctccgggtggcggccgccggagtagacgttagccatggaaaccgagagctggcccgggcggggccgcggtgagctcgttattcggccgccgcagcttttctgcctccgcattcgggcactaaccaacctcccggcgggagcgcccagcccgagtttacctgcaaaaatgcggtccctgggatgccttcgcgtcttctcttccctcgggtgacttgagaaactgctgtgttacagaaaagcatgtgactttcagaataatcccgagtgaggatgagtccagatgtgcctctactgaatgattacaagcaggacttctttctgaagcgctttccacagactgttcttggaggccctcgattcaaattaggctattgtgcccctccttacatatatgttaatcaaattattctttttctaatgccatgggtttggggtggagtcggaacacttttataccagttaggcatcctgaaagactattatacagcagcactttcaggtggattaatgctttttactgcatttgtcatccagttcacaagtttatacgccaaaaacaaatcaacaacagtagaaagaatactaaccacggatatcttagcagaggaggatgagcatgaatttaccagttgtactggtgctgagactgtcaaatttctcattcctggcaagaaatatgtagccaatacagtttttcattctattcttgctggattagcgtgtggtcttggaacatggtatctgctcccaaatagaataaccttgctgtatggcagtacaggaggcactgctctactattcttctttggatggatgacactatgtatagcagaatattctttaattgtaaacacagctacagagactgcgactttccaaacacaggatacttatgaaattattcctcttatgagacctctttatatttttttctttgtttctgtggatctggcacacaggtttgtggtaaatatgccagctctagaacacatgaatcagattttacacatcttgtttgtatttttaccctttctgtgggcacttgggactctgcccccacccgatgcacttctcttatgggcaatggagcaggttttagagttcggccttggaggctcatctatgtcaacccacttacggttattagtaatgttcatcatgtctgctggaacagctatagcatcatatttcattccaagcactgttggtgtggttcttttcatgactggatttggtttcttgctgagtctgaacttaagtgatatgggtcacaaaattggaaccaaatctaaggatttacccagtggtccggaaaaacatttttcatggaaggaatgccttttctacatcattatattagtcttggctcttttagaaactagcttgcttcatcactttgctggcttctcacagatttctaaaagcaattcccaggctattgtgggctatggtttgatgatattacttataatactgtggatacttagagaaattcaaagcgtatatatcattggaattttccgaaatcccttttatccgaaggatgtgcaaactgtgactgtattctttgagaagcaaactaggctcatgaagattggtattgtcagacggattttgctaactttagtatcaccttttgccatgatagcatttctttcattggacagttccttacaagggctccactcagtgtctgtctgtattggattcacaagagcctttagaatggtatggcagaatacagaaaatgctttattggagacagtcattgtatcaacagtacacttgatctccagtacagacatatggtggaacagaagcctggatacaggactcagactcttactggttggtatcatacgtgatcgtttgattcagttcatctctaaattgcagtttgccgtgactgtgcttttgacatcatggacagagaaaaaacaacgtcgaaaaacaactgccactttatgtatactcaacattgtcttttctccattcgtgttggtcatcatagttttttctacactactctcttctcccttactccctcttttcacccttcctgtgttcttggtggggtttccccgacctattcagagttggccaggagcagcaggcaccacagcctgtgtgtgtgcagatacagtgtactactaccaaatggtgcccaggttgactgctgtactgcagactgcaatggcagctggaagtttaggtctcctcctacctggatctcattacttgggccgttttcaggatcgtttaatgtggataatgattctggaatgtggctatacttactgctctattaacattaaggggttagaattgcaggaaacatcctgtcatactgcagaagctcgcagagttgatgaagtttttgaagatgcttttgagcaagaatacacaagagtatgttcccttaatgaacactttggaaatgtcttgacaccctgtactgttttgcctgtgaaattgtattctgatgccaggaatgttctatcaggcataattgattctcatgaaaacttaaaagaatttaaaggtgacctcattaaagtacttgtgtggatacttgttcaatactgctccaaaaggcctggcatgaaagagaatgttcacaacactgaaaataaagggaaagcacctctaatgttgcctgctttgaacactttgccacctcccaaatccccagaagacatagacagtttaaattcagaaacttttaatgactggtctgatgataatatttttgatgatgagccaactatcaaaaaagtaatagaagaaaaacatcagttgaaagatttgccaggtacaaatttgtttattccaggatcagtagaatcacagagggttggtgatcattctacaggcactgttcctgaaaacgatctttacaaagcagttctattaggataccctgctgttgacaaaggaaaacaagaggacatgccatatattcctctcatggagttcagttgttcacattctcacttagtatgcttacccgcagagtggaggactagctgtatgcccagttccaaaatgaaggagatgagctcgttatttccagaagactggtaccaatttgttctaaggcagttggaatgttatcattcagaagagaaggcctcaaatgtactggaagaaattgccaaggacaaagttttaaaagacttttatgttcatacagtaatgacttgttattttagtttatttggaatagacaatatggctcctagtcctggtcatatattgagagtttacggtggtgttttgccttggtctgttgctttggactggctcacagaaaagccagaactgtttcaactagcactgaaagcattcaggtatactctgaaactaatgattgataaagcaagtttaggtccaatagaagactttagagaactgattaagtaccttgaagaatatgaacgtgactggtacattggtttggtatctgatgaaaagtggaaggaagcaattttacaagaaaagccatacttgttttctctggggtatgattctaatatgggaatttacactgggagagtgcttagccttcaagaattattgatccaagtgggaaagttaaatcctgaagctgttagaggtcagtgggccaatctttcatgggaattactttatgccacaaacgatgatgaagaacgttatagtatacaagctcatccactacttttaagaaatcttacggtacaagcagcagaacctcccctgggatatccgatttattcttcaaaacctctccacatacatttgtattagagctcattttgactgtaatgtcatcaaatgcaatgtttttattttttcatcctaaaaaagtaactgtgattcttgtaacttgaggacttctccacacccccattcagatgcctgagaacagctaagctccgtaaagttggttctcttagccatcttaatggttctaaaaaacagcaaaaacatctttatgtctaagataaaagaactatttggccaatatttgtgccctctggactttagtaggctttggtaaatgtgagaaaacttttgtagaattatcatataatgaattttgtaatgctttcttaaatgtgttataggtgaattgccatacaaagttaacagctatgtaatttttacatacttaagagataaacatatcagtgttctaagtagtgataatggatcctgttgaaggttaacataatgtgtatatatttgtttgaaatataatttatagtattttcaaatgtgctgatttattttgacatctaatatctgaatgtttttgtatcaagtagtttgttttcatagacttcaattcataaactttaaaaaacttttaataaaatattttccttccttttcaaata

SEQ ID NO: 36 - DNA, 3513 bp, Homo sapiens
aaaggcccggcccaagggaacgttcagggcgtctcggctttccccgctgctgcttctgctaggcccagtgcgagaccagagcacgagcgactcccgtcgtccccggccaggcagatgttggcctagtcctggcgcgaacgaagcgcgctatttccctgcttcctctaggccaagcctgctttacggcagggcccgcctcgggagcgagcacagaccggggcagcgaggccagccaggcgccgacgaggtccccgaacgcgcacgcgctccgttcagctccgggtggcggccgccggagtagacgttagccatggaaaccgagagctggcccgggcggggccgcggtgagctcgttattcggccgccgcagcttttctgcctccgcattcgggcactaaccaacctcccggcgggagcgcccagcccgagtttacctgcaaaaatgcggtccctgggatgccttcgcgtcttctcttccctcgggtgacttgaggtttgtggtaaatatgccagctctagaacacatgaatcagattttacacatcttgtttgtatttttaccctttctgtgggcacttgggactctgcccccacccgatgcacttctcttatgggcaatggagcaggttttagagttcggccttggaggctcatctatgtcaacccacttacggttattagtaatgttcatcatgtctgctggaacagctatagcatcatatttcattccaagcactgttggtgtggttcttttcatgactggatttggtttcttgctgagtctgaacttaagtgatatgggtcacaaaattggaaccaaatctaaggatttacccagtggtccggaaaaacatttttcatggaaggaatgccttttctacatcattatattagtcttggctcttttagaaactagcttgcttcatcactttgctggcttctcacagatttctaaaagcaattcccaggctattgtgggctatggtttgatgatattacttataatactgtggatacttagagaaattcaaagcgtatatatcattggaattttccgaaatcccttttatccgaaggatgtgcaaactgtgactgtattctttgagaagcaaactaggctcatgaagattggtattgtcagacggattttgctaactttagtatcaccttttgccatgatagcatttctttcattggacagttccttacaagggctccactcagtgtctgtctgtattggattcacaagagcctttagaatggtatggcagaatacagaaaatgctttattggagacagtcattgtatcaacagtacacttgatctccagtacagacatatggtggaacagaagcctggatacaggactcagactcttactggttggtatcatacgtgatcgtttgattcagttcatctctaaattgcagtttgccgtgactgtgcttttgacatcatggacagagaaaaaacaacgtcgaaaaacaactgccactttatgtatactcaacattgtcttttctccattcgtgttggtcatcatagttttttctacactactctcttctcccttactccctcttttcacccttcctgtgttcttggtggggtttccccgacctattcagagttggccaggagcagcaggcaccacagcctgtgtgtgtgcagatacagtgtactactaccaaatggtgcccaggttgactgctgtactgcagactgcaatggcagctggaagtttaggtctcctcctacctggatctcattacttgggccgttttcaggatcgtttaatgtggataatgattctggaatgtggctatacttactgctctattaacattaaggggttagaattgcaggaaacatcctgtcatactgcagaagctcgcagagttgatgaagtttttgaagatgcttttgagcaagaatacacaagagtatgttcccttaatgaacactttggaaatgtcttgacaccctgtactgttttgcctgtgaaattgtattctgatgccaggaatgttctatcaggcataattgattctcatgaaaacttaaaagaatttaaaggtgacctcattaaagtacttgtgtggatacttgttcaatactgctccaaaaggcctggcatgaaagagaatgttcacaacactgaaaataaagggaaagcacctctaatgttgcctgctttgaacactttgccacctcccaaatccccagaagacatagacagtttaaattcagaaacttttaatgactggtctgatgataatatttttgatgatgagccaactatcaaaaaagtaatagaagaaaaacatcagttgaaagatttgccaggtacaaatttgtttattccaggatcagtagaatcacagagggttggtgatcattctacaggcactgttcctgaaaacgatctttacaaagcagttctattaggataccctgctgttgacaaaggaaaacaagaggacatgccatatattcctctcatggagttcagttgttcacattctcacttagtatgcttacccgcagagtggaggactagctgtatgcccagttccaaaatgaaggagatgagctcgttatttccagaagactggtaccaatttgttctaaggcagttggaatgttatcattcagaagagaaggcctcaaatgtactggaagaaattgccaaggacaaagttttaaaagacttttatgttcatacagtaatgacttgttattttagtttatttggaatagacaatatggctcctagtcctggtcatatattgagagtttacggtggtgttttgccttggtctgttgctttggactggctcacagaaaagccagaactgtttcaactagcactgaaagcattcaggtatactctgaaactaatgattgataaagcaagtttaggtccaatagaagactttagagaactgattaagtaccttgaagaatatgaacgtgactggtacattggtttggtatctgatgaaaagtggaaggaagcaattttacaagaaaagccatacttgttttctctggggtatgattctaatatgccaggaccagccttggagataagcagagtgaacagaaatttatggtctcagatttaagaaaaacaaaattctttcttgcttcttaaatcatactccatcccattggcttgcaaacatgctgacactcctcataatttcactcttcataaaccaaagcataaggtcagaggagaacttgacatattagaacacttaggcattgaaagtggttagtctaactaacccattgaagttttggagaacctgggactcaaattttggaagatgtgacagatgatatgttaacatacattgcaccgaggctgaagtgggaggattgcttgagactgcctggaaggcagaagttgcagtgagccgagactgatggtgtcactgcactccagcctgggcaacagagcaagaccctgtcttaaaaaaacaaaacaaacaaacaaaagaaacc

SEQ ID NO: 37 - protein, 406 aa, Homo sapiens
MDCRTKANPDRTFDLVLKVKCHASENEDPVVLWKFPEDFGDQEILQSVPKFCFPFDVERVSQNQVGQHFTFVLTDIESKQRFGFCRLTSGGTICLCILSYLPWFEVYYKLLNTLADYLAKELENDLNETLRSLYNHPVPKANTPVNLSVHSYFIAPDVTGLPTIPESRNLTEYFVAVDVNNMLQLYASMLHERRIVIISSKLSTLTACIHGSAALLYPMYWQHIYIPVLPPHLLDYCCAPMPYLIGIHSSLIERVKNKSLEDVVMLNVDTNTLESPFSDLNNLPSDVVSALKNKLKKQSTATGDGVARAFLRAQAALFGSYRDALRYKPGEPITFCEESFVKHRSSVMKQFLETAINLQLFKQFIDGRLAKLNAGRGFSDVFEEEITSGGFCGGKDKLQYDYPFSQ

SEQ ID NO: 38 - protein, 426 aa, Homo sapiens
MDCRTKANPDRTFDLVLKVKCHASENEDPVVLWKFPEDFGDQEILQSVPKFCFPFDVERVSQNQVGQHFTFVLTDIESKQRFGFCRLTSGGTICLCILSYLPWFEVYYKLLNTLADYLAKELENDLNETLRSLYNHPVPKANTPVNLSVNQEIFIACEQVLKDQPALVPHSYFIAPDVTGLPTIPESRNLTEYFVAVDVNNMLQLYASMLHERRIVIISSKLSTLTACIHGSAALLYPMYWQHIYIPVLPPHLLDYCCAPMPYLIGIHSSLIERVKNKSLEDVVMLNVDTNTLESPFSDLNNLPSDVVSALKNKLKKQSTATGDGVARAFLRAQAALFGSYRDALRYKPGEPITFCEESFVKHRSSVMKQFLETAINLQLFKQFIDGRLAKLNAGRGFSDVFEEEITSGGFCGGKDKLQYDYPFSQ

SEQ ID NO: 39 - protein, 426 aa, Homo sapiens
IETKTRANPDRTFDLVLKVKCHASENEDPVVLWKFPEDFGDQEILQSVPKFCFPFDVERVSQNQVGQHFTFVLTDIESKQRFGFCRLTSGGTICLCILSYLPWFEVYYKLLNTLADYLAKELENDLNETLRSLYNHPVPKANTPVNLSVNQEIFIACEQVLKDQPALVPHSYFIAPDVTGLPTIPESRNLTEYFVAVDVNNMLQLYASMLHERRIVIISSKLSTLTACIHGSAALLYPMYWQHIYIPVLPPHLLDYCCAPMPYLIGIHSSLIERVKNKSLEDVVMLNVDTNTLESPFSDLNNLPSDVVSALKNKLKKQSTATGDGVARAFLRAQAALFGSYRDALRYKPGEPITFCEESFVKHRSSVMKQFLETAINLQLFKQFIDGRLAKLNAGRGFSDVFEEEITSGGFCGGKDKLQYDYPFSQ

SEQ ID NO: 40 - protein, 396 aa, Homo sapiens
MAAAPREEKRWPQPVFSNPVVLWKFPEDFGDQEILQSVPKFCFPFDVERVSQNQVGQHFTFVLTDIESKQRFGFCRLTSGGTICLCILSYLPWFEVYYKLLNTLADYLAKELENDLNETLRSLYNHPVPKANTPVNLSVHSYFIAPDVTGLPTIPESRNLTEYFVAVDVNNMLQLYASMLHERRIVIISSKLSTLTACIHGSAALLYPMYWQHIYIPVLPPHLLDYCCAPMPYLIGIHSSLIERVKNKSLEDVVMLNVDTNTLESPFSDLNNLPSDVVSALKNKLKKQSTATGDGVARAFLRAQAALFGSYRDALRYKPGEPITFCEESFVKHRSSVMKQFLETAINLQLFKQFIDGRLAKLNAGRGFSDVFEEEITSGGFCGGKDKLQYDYPFSQ

SEQ ID NO: 41 - DNA, 2117 bp, Homo sapiens
gccgggggcgcagccgacatgggcccgccgccacggctgctgtgagcagcctctttccctgtgtggccgccggcgtgggcggggacggcgcgaccctcgcgcggccgggctgcgggcttccaggccagcgcgcgggggccggacggacagccccacaccgacatgtaaccatggactgcaggaccaaggcaaatccagacagaacctttgacttggtgttgaaagtgaaatgtcatgcctctgaaaatgaagatcctgtggtattgtggaaattcccagaggactttggagaccaggaaatactacagagtgtgccaaagttctgttttccctttgacgttgaaagggtgtctcagaatcaagttggacagcactttacctttgtactgacagacattgaaagtaaacagagatttggattctgcagactgacgtcaggaggcacaatttgtttatgcatccttagttaccttccctggtttgaagtgtattacaagcttctaaatactcttgcagattacttggctaaggaactggaaaatgatttgaatgaaactctcagatcactgtataaccacccagtaccaaaggcaaatactcctgtaaatttgagtgtgcattcctacttcattgcccctgatgtaactggactcccaacaatacccgagagtagaaatcttacagaatattttgttgccgtggatgtgaacaacatgctgcagctgtatgccagtatgctgcatgaaaggcgcatcgtgattatctcgagcaaattaagcactttaactgcctgtatccatggatcagctgctcttctatacccaatgtattggcaacacatatacatcccagtgcttcctccacacctgctggactactgctgtgccccaatgccatacctgattggaatacactccagcctcatagagagagtgaaaaacaaatcattggaagatgttgttatgttaaatgttgatacaaacacattagaatcaccatttagtgacttgaacaacctaccaagtgatgtggtctcggccttgaaaaataaactgaagaagcagtctacagctacgggtgatggagtagctagggcctttcttagagcacaggctgctttgtttggatcctacagagatgcactgagatacaaacctggtgagcccatcactttctgtgaggagagttttgtaaagcaccgctcaagcgtgatgaaacagttcctggaaactgccattaacctccagctttttaagcagtttatcgatggtcgactggcaaaactaaatgcaggaaggggtttctctgatgtatttgaagaagagatcacttcaggtggcttttgtggaggtaaagacaagttacaatatgattatccattttctcaataacaattttcttggtctttgcacttgtgtctgataaaacctatttcataaacaactaatgatttcctcctaaatatgtaatgtcttaaatacatttttcatcttataaaagctatggaattagcttattttgcctgatacctgttactcaaggcattaagttggcctcctgaattggcagctgttggcctcgataatctcttaatattgctggaaattagtaatacagaaatccaatcaactcatatcttcctgtctttccttctgaatagtagtattctctgctagaaaactactagtgatggttattactgagtatgaatttaagaactgaggttatgattggtaatacaatccaaaaagaagggtctgaacaccaaaattctttatacatatttaagtaactgtattattattatacagatgtctttacctttttgactttatagatcactgcagcattaagaaagtttccagtttaccattccataagtacaattaatccttctagtgtaaatgttcaaatactgttataattatctaggcaattaataatttacaaactgatatttttgcacgattgtagtggtgtatagtcttgacttgcagagcattttgcttgagtccttgaaatgtcgtgttcattcattatttgctgagtgcttacaatgtattaggcactgttctaaatattaagtgtactaaataaacaaaaatccttgtattctg

SEQ ID NO: 42 - DNA, 2177 bp, Homo sapiens
gccgggggcgcagccgacatgggcccgccgccacggctgctgtgagcagcctctttccctgtgtggccgccggcgtgggcggggacggcgcgaccctcgcgcggccgggctgcgggcttccaggccagcgcgcgggggccggacggacagccccacaccgacatgtaaccatggactgcaggaccaaggcaaatccagacagaacctttgacttggtgttgaaagtgaaatgtcatgcctctgaaaatgaagatcctgtggtattgtggaaattcccagaggactttggagaccaggaaatactacagagtgtgccaaagttctgttttccctttgacgttgaaagggtgtctcagaatcaagttggacagcactttacctttgtactgacagacattgaaagtaaacagagatttggattctgcagactgacgtcaggaggcacaatttgtttatgcatccttagttaccttccctggtttgaagtgtattacaagcttctaaatactcttgcagattacttggctaaggaactggaaaatgatttgaatgaaactctcagatcactgtataaccacccagtaccaaaggcaaatactcctgtaaatttgagtgtgaaccaagagatatttattgcctgtgagcaagttctgaaagatcagcctgctctagtaccgcattcctacttcattgcccctgatgtaactggactcccaacaatacccgagagtagaaatcttacagaatattttgttgccgtggatgtgaacaacatgctgcagctgtatgccagtatgctgcatgaaaggcgcatcgtgattatctcgagcaaattaagcactttaactgcctgtatccatggatcagctgctcttctatacccaatgtattggcaacacatatacatcccagtgcttcctccacacctgctggactactgctgtgccccaatgccatacctgattggaatacactccagcctcatagagagagtgaaaaacaaatcattggaagatgttgttatgttaaatgttgatacaaacacattagaatcaccatttagtgacttgaacaacctaccaagtgatgtggtctcggccttgaaaaataaactgaagaagcagtctacagctacgggtgatggagtagctagggcctttcttagagcacaggctgctttgtttggatcctacagagatgcactgagatacaaacctggtgagcccatcactttctgtgaggagagttttgtaaagcaccgctcaagcgtgatgaaacagttcctggaaactgccattaacctccagctttttaagcagtttatcgatggtcgactggcaaaactaaatgcaggaaggggtttctctgatgtatttgaagaagagatcacttcaggtggcttttgtggaggtaaagacaagttacaatatgattatccattttctcaataacaattttcttggtctttgcacttgtgtctgataaaacctatttcataaacaactaatgatttcctcctaaatatgtaatgtcttaaatacatttttcatcttataaaagctatggaattagcttattttgcctgatacctgttactcaaggcattaagttggcctcctgaattggcagctgttggcctcgataatctcttaatattgctggaaattagtaatacagaaatccaatcaactcatatcttcctgtctttccttctgaatagtagtattctctgctagaaaactactagtgatggttattactgagtatgaatttaagaactgaggttatgattggtaatacaatccaaaaagaagggtctgaacaccaaaattctttatacatatttaagtaactgtattattattatacagatgtctttacctttttgactttatagatcactgcagcattaagaaagtttccagtttaccattccataagtacaattaatccttctagtgtaaatgttcaaatactgttataattatctaggcaattaataatttacaaactgatatttttgcacgattgtagtggtgtatagtcttgacttgcagagcattttgcttgagtccttgaaatgtcgtgttcattcattatttgctgagtgcttacaatgtattaggcactgttctaaatattaagtgtactaaataaacaaaaatccttgtattctg

SEQ ID NO: 43 - DNA, 2007 bp, Homo sapiens
attgagacaaaaacaagggcaaatccagacagaacctttgacttggtgttgaaagtgaaatgtcatgcctctgaaaatgaagatcctgtggtattgtggaaattcccagaggactttggagaccaggaaatactacagagtgtgccaaagttctgttttccctttgacgttgaaagggtgtctcagaatcaagttggacagcactttacctttgtactgacagacattgaaagtaaacagagatttggattctgcagactgacgtcaggaggcacaatttgtttatgcatccttagttaccttccctggtttgaagtgtattacaagcttctaaatactcttgcagattacttggctaaggaactggaaaatgatttgaatgaaactctcagatcactgtataaccacccagtaccaaaggcaaatactcctgtaaatttgagtgtgaaccaagagatatttattgcctgtgagcaagttctgaaagatcagcctgctctagtaccgcattcctacttcattgcccctgatgtaactggactcccaacaatacccgagagtagaaatcttacagaatattttgttgccgtggatgtgaacaacatgctgcagctgtatgccagtatgctgcatgaaaggcgcatcgtgattatctcgagcaaattaagcactttaactgcctgtatccatggatcagctgctcttctatacccaatgtattggcaacacatatacatcccagtgcttcctccacacctgctggactactgctgtgccccaatgccatacctgattggaatacactccagcctcatagagagagtgaaaaacaaatcattggaagatgttgttatgttaaatgttgatacaaacacattagaatcaccatttagtgacttgaacaacctaccaagtgatgtggtctcggccttgaaaaataaactgaagaagcagtctacagctacgggtgatggagtagctagggcctttcttagagcacaggctgctttgtttggatcctacagagatgcactgagatacaaacctggtgagcccatcactttctgtgaggagagttttgtaaagcaccgctcaagcgtgatgaaacagttcctggaaactgccattaacctccagctttttaagcagtttatcgatggtcgactggcaaaactaaatgcaggaaggggtttctctgatgtatttgaagaagagatcacttcaggtggcttttgtggaggtaaagacaagttacaatatgattatccattttctcaataacaattttcttggtctttgcacttgtgtctgataaaacctatttcataaacaactaatgatttcctcctaaatatgtaatgtcttaaatacatttttcatcttataaaagctatggaattagcttattttgcctgatacctgttactcaaggcattaagttggcctcctgaattggcagctgttggcctcgataatctcttaatattgctggaaattagtaatacagaaatccaatcaactcatatcttcctgtctttccttctgaatagtagtattctctgctagaaaactactagtgatggttattactgagtatgaatttaagaactgaggttatgattggtaatacaatccaaaaagaagggtctgaacaccaaaattctttatacatatttaagtaactgtattattattatacagatgtctttacctttttgactttatagatcactgcagcattaagaaagtttccagtttaccattccataagtacaattaatccttctagtgtaaatgttcaaatactgttataattatctaggcaattaataatttacaaactgatatttttgcacgattgtagtggtgtatagtcttgacttgcagagcattttgcttgagtccttgaaatgtcgtgttcattcattatttgctgagtgcttacaatgtattaggcactgttctaaatattaagtgtactaaataaacaaaaatccttgtattctg

SEQ ID NO: 44 - DNA, 2197 bp, Homo sapiens
gcgggggccggacggacagccccacaccgacatgtaaccatggactgcaggaccaaggcaaatccagacagaacctttgacttggtgttgaaagtgaaatgtcatgcctctgaaaatgaagaggacagtccagcttatctgccgaggattccccctggaaaagtacgccgattcgcattttgcattaagaaactggaaaactttcctgtcggtcctggcgtagcgcctcccgtgtccggggtagaccttgtaccggctgaaaccgcatagctcgaccttcatggcggcagctccacgggaggagaaaagatggccccaacctgtattttcgaatcctgtggtattgtggaaattcccagaggactttggagaccaggaaatactacagagtgtgccaaagttctgttttccctttgacgttgaaagggtgtctcagaatcaagttggacagcactttacctttgtactgacagacattgaaagtaaacagagatttggattctgcagactgacgtcaggaggcacaatttgtttatgcatccttagttaccttccctggtttgaagtgtattacaagcttctaaatactcttgcagattacttggctaaggaactggaaaatgatttgaatgaaactctcagatcactgtataaccacccagtaccaaaggcaaatactcctgtaaatttgagtgtgcattcctacttcattgcccctgatgtaactggactcccaacaatacccgagagtagaaatcttacagaatattttgttgccgtggatgtgaacaacatgctgcagctgtatgccagtatgctgcatgaaaggcgcatcgtgattatctcgagcaaattaagcactttaactgcctgtatccatggatcagctgctcttctatacccaatgtattggcaacacatatacatcccagtgcttcctccacacctgctggactactgctgtgccccaatgccatacctgattggaatacactccagcctcatagagagagtgaaaaacaaatcattggaagatgttgttatgttaaatgttgatacaaacacattagaatcaccatttagtgacttgaacaacctaccaagtgatgtggtctcggccttgaaaaataaactgaagaagcagtctacagctacgggtgatggagtagctagggcctttcttagagcacaggctgctttgtttggatcctacagagatgcactgagatacaaacctggtgagcccatcactttctgtgaggagagttttgtaaagcaccgctcaagcgtgatgaaacagttcctggaaactgccattaacctccagctttttaagcagtttatcgatggtcgactggcaaaactaaatgcaggaaggggtttctctgatgtatttgaagaagagatcacttcaggtggcttttgtggaggtaaagacaagttacaatatgattatccattttctcaataacaattttcttggtctttgcacttgtgtctgataaaacctatttcataaacaactaatgatttcctcctaaatatgtaatgtcttaaatacatttttcatcttataaaagctatggaattagcttattttgcctgatacctgttactcaaggcattaagttggcctcctgaattggcagctgttggcctcgataatctcttaatattgctggaaattagtaatacagaaatccaatcaactcatatcttcctgtctttccttctgaatagtagtattctctgctagaaaactactagtgatggttattactgagtatgaatttaagaactgaggttatgattggtaatacaatccaaaaagaagggtctgaacaccaaaattctttatacatatttaagtaactgtattattattatacagatgtctttacctttttgactttatagatcactgcagcattaagaaagtttccagtttaccattccataagtacaattaatccttctagtgtaaatgttcaaatactgttataattatctaggcaattaataatttacaaactgatatttttgcacgattgtagtggtgtatagtcttgacttgcagagcattttgcttgagtccttgaaatgtcgtgttcattcattatttgctgagtgcttacaatgtattaggcactgttctaaatattaagtgtactaaataaacaaaaatccttgtattctg

SEQ ID NO: 45 - protein, 423 aa, Homo sapiens
MGGPRAWALLCLGLLLPGGGAAWSIGAAPFSGRRNWCSYVVTRTISCHVQNGTYLQRVLQNCPWPMSCPGSRTVVRPTYKVMYKIVTAPSSASLEPMWSGSTMRRMALRPTAFSGCLNCSKVSELTERLKVLEAKMTMLTVIEQPVPPTPATPEDPAPLWGPPPAQGSPGDGGLQDQVGAWGLPGPTGPKGDAGSRGPMGMRGPPGPQGPPGSPGRAGAVGTPGERGPPGPPGPPGPPGPPAPVGPPHARISQHGDPLLSNTFTETNNHWPQGPTGPPGPPGPMGPPGPPGPTGVPGSPGHIGPPGPTGPKGISGHPGEKGERGLRGEPGPQGSAGQRGEPGPKGDPGEKSHWGEGLHQLREALKILAERVLILETMIGLYEPELGSGAGPAGTGTPSLLRGKRGGHATNYRIVAPRSRDERG

SEQ ID NO: 46 - protein, 212 aa, Homo sapiens
MGGPRAWALLCLGLLLPGGGAAWSIGAAPFSGRRNWCSYVVTRTISCHVQNGTYLQRVLQNCPWPMSCPGSSYRTVVRPTYKVMYKIVTAREWRCCPGHSGVSCEEVAASSASLEPMWSGSTMRRMALRPTAFSGCLNCSKVSELTERLKVLEAKMTMLTVIEQPVPPTPATPEDPAPLWGPPPAQGSPGDGGLQGDPLLSNTFTETNNHWP

SEQ ID NO: 47 - protein, 175 aa, Homo sapiens
MGGPRAWALLCLGLLLPGGGAAWSIGAAPFSGRRNWCSYVVTRTISCHVQNGTYLQRVLQNCPWPMSCPGSSYRTVVRPTYKVMYKIVTAREWRCCPGHSGVSCEEGCLNCSKVSELTERLKVLEAKMTMLTVIEQPVPPTPATPEDPAPLWGPPPAQGSPGDGGLQDQVGAWGL

SEQ ID NO: 48 - protein, 34 aa, Homo sapiens
MGGPRAWALLCLGLLLPGGGAAWSIGAAPFSGRR

SEQ ID NO: 49 - protein, 445 aa, Homo sapiens
MGGPRAWALLCLGLLLPGGGAAWSIGAAPFSGRRNWCSYVVTRTISCHVQNGTYLQRVLQNCPWPMSCPGSSYRTVVRPTYKVMYKIVTAREWRCCPGHSGVSCEEVAASSASLEPMWSGSTMRRMALRPTAFSGCLNCSKVSELTERLKVLEAKMTMLTVIEQPVPPTPATPEDPAPLWGPPPAQGSPGDGGLQDQVGAWGLPGPTGPKGDAGSRGPMGMRGPPGPQGPPGSPGRAGAVGTPGERGPPGPPGPPGPPGPPAPVGPPHARISQHGDPLLSNTFTETNNHWPQGPTGPPGPPGPMGPPGPPGPTGVPGSPGHIGPPGPTGPKGISGHPGEKGERGLRGEPGPQGSAGQRGEPGPKGDPGEKSHWAPSLQSFLQQQAQLELLARRVTLLEAIIWPEPELGSGAGPAGTGTPSLLRGKRGGHATNYRIVAPRSRDERG

SEQ ID NO: 50 - protein, 443 aa, Homo sapiens
MGGPRAWALLCLGLLLPGGGAAWSIGAAPFSGRRNWCSYVVTRTISCHVQNGTYLQRVLQNCPWPMSCPGSSYRTVVRPTYKVMYKIVTAREWRCCPGHSGVSCEEVAASSASLEPMWSGSTMRRMALRPTAFSGCLNCSKVSELTERLKVLEAKMTMLTVIEQPVPPTPATPEDPAPLWGPPPAQGSPGDGGLQDQVGAWGLPGPTGPKGDAGSRGPMGMRGPPGPQGPPGSPGRAGAVGTPGERGPPGPPGPPGPPGPPAPVGPPHARISQHGDPLLSNTFTETNNHWPQGPTGPPGPPGPMGPPGPPGPTGVPGSPGHIGPPGPTGPKGISGHPGEKGERGLRGEPGPQGSAGQRGEPGPKGDPGEKSHWGEGLHQLREALKILAERVLILETMIGLYEPELGSGAGPAGTGTPSLLRGKRGGHATNYRIVAPRSRDERG

SEQ ID NO: 51 - protein, 441 aa, Homo sapiens
MGGPRAWALLCLGLLLPGGGAAWSIGAAPFSGRRNWCSYVVTRTISCHVQNGTYLQRVLQNCPWPMSCPGSSYRTVVRPTYKVMYKIVTAREWRCCPGHSGVSCEEASSASLEPMWSGSTMRRMALRPTAFSGCLNCSKVSELTERLKVLEAKMTMLTVIEQPVPPTPATPEDPAPLWGPPPAQGSPGDGGLQDQVGAWGLPGPTGPKGDAGSRGPMGMRGPPGPQGPPGSPGRAGAVGTPGERGPPGPPGPPGPPGPPAPVGPPHARISQHGDPLLSNTFTETNNHWPQGPTGPPGPPGPMGPPGPPGPTGVPGSPGHIGPPGPTGPKGISGHPGEKGERGLRGEPGPQGSAGQRGEPGPKGDPGEKSHWGEGLHQLREALKILAERVLILETMIGLYEPELGSGAGPAGTGTPSLLRGKRGGHATNYRIVAPRSRDERG

SEQ ID NO: 52 - protein, 439 aa, Homo sapiens
MGGPRAWALLCLGLLLPGGGAAWSIGAAPFSGRRNWCSYVVTRTISCHVQNGTYLQRVLQNCPWPMSCPGSRTVVRPTYKVMYKIVTAREWRCCPGHSGVSCEEASSASLEPMWSGSTMRRMALRPTAFSGCLNCSKVSELTERLKVLEAKMTMLTVIEQPVPPTPATPEDPAPLWGPPPAQGSPGDGGLQDQVGAWGLPGPTGPKGDAGSRGPMGMRGPPGPQGPPGSPGRAGAVGTPGERGPPGPPGPPGPPGPPAPVGPPHARISQHGDPLLSNTFTETNNHWPQGPTGPPGPPGPMGPPGPPGPTGVPGSPGHIGPPGPTGPKGISGHPGEKGERGLRGEPGPQGSAGQRGEPGPKGDPGEKSHWGEGLHQLREALKILAERVLILETMIGLYEPELGSGAGPAGTGTPSLLRGKRGGHATNYRIVAPRSRDERG

SEQ ID NO: 53 - protein, 422 aa, Homo sapiens
MGGPRAWALLCLGLLLPGGGAAWSIGAAPFSGRRNWCSYVVTRTISCHVQNGTYLQRVLQNCPWPMSCPGSSYRTVVRPTYKVMYKIVTAREWRCCPGHSGVSCEEVAASSASLEPMWSGSTMRRMALRPTAFSGCLNCSKVSELTERLKVLEAKMTMLTVIEQPVPPTPATPEDPAPLWGPPPAQGSPGDGGLQDQVGAWGLPGPTGPKGDAGSRGPMGMRGPPGPQGPPGSPGRAGAVGTPGERGPPGPPGPPGPPGPPAPVGPPHARISQHGDPLLSNTFTETNNHWPQGPTGPPGPPGPMGPPGPPGPTGVPGSPGHIGLRGEPGPQGSAGQRGEPGPKGDPGEKSHWGEGLHQLREALKILAERVLILETMIGLYEPELGSGAGPAGTGTPSLLRGKRGGHATNYRIVAPRSRDERG

SEQ ID NO: 54 - protein, 212 aa, Homo sapiens; identical to SEQ ID NO: 46

SEQ ID NO: 55 - protein, 175 aa, Homo sapiens; identical to SEQ ID NO: 47

SEQ ID NO: 56 - protein, 236 aa, Homo sapiens
  misc_feature (1)..(1): Xaa can be any naturally occurring amino acid
XMTMLTVIEQPVPPTPATPEDPAPLWGPPPAQGSPGDGGLQGLPGAIESVRVPLLPRNDQVGAWGLPGPTGPKGDAGSRGPMGMRGPPGPQGPPGSPGRAGAVGTPGERGPPGPPGPPGPPGPPAPVGPPHARISQHGDPLLSNTFTETNNHWPQGPTGPPGPPGPMGPPGPPGPTGVPGSPGHIGPPGPTGPKGISGHPGEKGERGLRGEPGPQGSAGQRGEPGPKGDPGEKSHW

SEQ ID NO: 57 - protein, 305 aa, Homo sapiens
MTMLTVIEQPVPPTPATPEDPAPLWGPPPAQGSPGDGGLQGLPGAIESVRVPLLPRNDQVGAWGLPGPTGPKGDAGSRGPMGMRGPPGPQGPPGSPGRAGAVGTPGERGPPGPPGPPGPPGPPAPVGPPHARISQHGDPLLSNTFTETNNHWPQGPTGPPGPPGPMGPPGPPGPTGVPGSPGHIGPPGPTGPKGISGHPGEKGERGLRGEPGPQGSAGQRGEPGPKGDPGEKSHWGEGLHQLREALKILAERVLILETMIGLYEPELGSGAGPAGTGTPSLLRGKRGGHATNYRIVAPRSRDERG

SEQ ID NO: 58 - protein, 226 aa, Homo sapiens [sequence omitted]

SEQ ID NO: 59 - DNA, 2031 bp, Homo sapiens
gggctccgcgcgtccggggcggctggcggcgcgggcaggcaggcggggaggacaggctgggggcggcgaccgcgaggggccgcgcgcggagggcgcctggtgcagcatgggcggcccgcgggcttgggcgctgctctgcctcgggctcctgctcccgggaggcggcgctgcgtggagcatcggggcagctccgttctccggacgcaggaactggtgctcctatgtggtgacccgcaccatctcatgccatgtgcagaatggcacctaccttcagcgagtgctgcagaactgcccctggcccatgagctgtccggggagcagaactgtggtgagacccacatacaaggtgatgtacaagatagtgaccgccccttcctctgcctccttggagcccatgtggtcgggcagtaccatgcggcggatggcgcttcggcccacagccttctcaggttgtctcaactgcagcaaagtgtcagagctgacagagcggctgaaggtgctggaggccaagatgaccatgctgactgtcatagagcagccagtacctccaacaccagctacccctgaggaccctgccccgctctggggtccccctcctgcccagggcagccccggagatggaggcctccaggaccaagtcggtgcttgggggcttcccgggcccaccggccccaagggagatgccggcagtcggggcccaatggggatgagaggcccaccaggtccacagggccccccagggagccctggccgggctggagctgtgggcacccctggagagaggggacctcctgggccaccagggcctcctggcccccctgggcccccagcccctgttgggccaccccatgcccggatctcccagcatggagacccattgctgtccaacaccttcactgagaccaacaaccactggccccagggacccactgggcctccaggccctccagggcccatgggtccccctgggcctcctggccccacaggtgtccctgggagtcctggtcacataggacccccaggccccactggacccaaaggaatctctggccacccaggagagaagggcgagagaggactgcgtggggagcctggcccccaaggctctgctgggcagcggggggaacctggccctaagggagaccctggtgagaagagccactggggggaggggttgcaccagctacgcgaggctttgaagattttagctgagagggttttaatcttggaaacaatgattgggctctatgaaccagagctggggtctggggcgggccctgccggcacaggcacccccagcctccttcggggcaagaggggcggacatgcaaccaactaccggatcgtggcccccaggagccgggacgagagaggctgagggtggtggcggcccctgaggcagaccaggccaggcttcccctcctacctggactcggccagctgcctccagggaccgcccgtccatatttattaatgtcctcagggtcccttctgccatctaggccttaggggtaagcaggtctcagtcctggcaccatgcacatgtctgaggctgagcaagggctgagaggagaggcttgggcctcagtttccctctgtgaagtggggggaggcaggccttcaaggagggatagaggtacaaggcttcgtctcatctgctgtctgagcatccaggcccaaaggcactgagggagtcaggagctggggctcggcacatgcagagatgacagggcagggggcagtcttcctccccctccccgaccaaacctcggggagccctcctgtgcccctccctccttgttgtccagtgctgggctccccaccccgaggtcaggctgcccaatcctctgactggatcaccgggggcttcttgcctcagttcttccctctgagcccccaggccctcccgcatctcaggttggggatggggacatggagaggaaggggccgcctactcctgcaaatgcttgtgacagatgccaggaggtagatgtgtgctggccaataaaggcccctacctgattccccgca

SEQ ID NO: 60 - DNA, 769 bp, Homo sapiens
cgccctccggccgcggagctggaaaccgggctccgcgcgtccggggcggctggcggcgcgggcaggcaggcggggaggacaggctgggggcggcgaccgcgaggggccgcgcgcggagggcgcctggtgcagcatgggcggcccgcgggcttgggcgctgctctgcctcgggctcctgctcccgggaggcggcgctgcgtggagcatcggggcagctccgttctccggacgcaggaactggtgctcctatgtggtgacccgcaccatctcatgccatgtgcagaatggcacctaccttcagcgagtgctgcagaactgcccctggcccatgagctgtccggggagcagctacagaactgtggtgagacccacatacaaggtgatgtacaagatagtgaccgcccgtgagtggaggtgctgccctgggcactcaggagtgagctgcgaggaagttgcagcttcctctgcctccttggagcccatgtggtcgggcagtaccatgcggcggatggcgcttcggcccacagccttctcaggttgtctcaactgcagcaaagtgtcagagctgacagagcggctgaaggtgctggaggccaagatgaccatgctgactgtcatagagcagccagtacctccaacaccagctacccctgaggaccctgccccgctctggggtccccctcctgcccagggcagccccggagatggaggcctccagggagacccattgctgtccaacaccttcactgagaccaacaaccactggccc

SEQ ID NO: 61 - DNA, 641 bp, Homo sapiens
gctggaaaccgggctccgcgcgtccggggcggctggcggcgcgggcaggcaggcggggaggacaggctgggggcggcgaccgcgaggggccgcgcgcggagggcgcctggtgcagcatgggcggcccgcgggcttgggcgctgctctgcctcgggctcctgctcccgggaggcggcgctgcgtggagcatcggggcagctccgttctccggacgcaggaactggtgctcctatgtggtgacccgcaccatctcatgccatgtgcagaatggcacctaccttcagcgagtgctgcagaactgcccctggcccatgagctgtccggggagcagctacagaactgtggtgagacccacatacaaggtgatgtacaagatagtgaccgcccgtgagtggaggtgctgccctgggcactcaggagtgagctgcgaggaaggttgtctcaactgcagcaaagtgtcagagctgacagagcggctgaaggtgctggaggccaagatgaccatgctgactgtcatagagcagccagtacctccaacaccagctacccctgaggaccctgccccgctctggggtccccctcctgcccagggcagccccggagatggaggcctccaggaccaagtcggtgcttgggggctt

SEQ ID NO: 62 - DNA, 482 bp, Homo sapiens
cggcgcgggcaggcaggcggggaggacaggctgggggcggcgaccgcgaggggccgcgcgcggagggcgcctggtgcagcatgggcggcccgcgggcttgggcgctgctctgcctcgggctcctgctcccgggaggcggcgctgcgtggagcatcggggcagctccgttctccggacgcagatgaccatgctgactgtcatagagcagccagtacctccaacaccagctacccctgaggaccctgccccgctctggggtccccctcctgcccagggcagccccggagatggaggcctccaggaccaagtcggtgcttgggggcttcccgggcccaccggccccaagggagatgccggcagtcggggcccaatggggatgagaggcccaccaggtccacagggccccccagggagccctggccgggctggagctgtgggcacccctggagagaggggacctcctgggccaccagggcctcctg

SEQ ID NO: 63 - DNA, 2066 bp, Homo sapiens
cgggcaggcaggcggggaggacaggctgggggcggcgaccgcgaggggccgcgcgcggagggcgcctggtgcagcatgggcggcccgcgggcttgggcgctgctctgcctcgggctcctgctcccgggaggcggcgctgcgtggagcatcggggcagctccgttctccggacgcaggaactggtgctcctatgtggtgacccgcaccatctcatgccatgtgcagaatggcacctaccttcagcgagtgctgcagaactgcccctggcccatgagctgtccggggagcagctacagaactgtggtgagacccacatacaaggtgatgtacaagatagtgaccgcccgtgagtggaggtgctgccctgggcactcaggagtgagctgcgaggaagttgcagcttcctctgcctccttggagcccatgtggtcgggcagtaccatgcggcggatggcgcttcggcccacagccttctcaggttgtctcaactgcagcaaagtgtcagagctgacagagcggctgaaggtgctggaggccaagatgaccatgctgactgtcatagagcagccagtacctccaacaccagctacccctgaggaccctgccccgctctggggtccccctcctgcccagggcagccccggagatggaggcctccaggaccaagtcggtgcttgggggcttcccgggcccaccggccccaagggagatgccggcagtcggggcccaatggggatgagaggcccaccaggtccacagggccccccagggagccctggccgggctggagctgtgggcacccctggagagaggggacctcctgggccaccagggcctcctggcccccctgggcccccagcccctgttgggccaccccatgcccggatctcccagcatggagacccattgctgtccaacaccttcactgagaccaacaaccactggccccagggacccactgggcctccaggccctccagggcccatgggtccccctgggcctcctggccccacaggtgtccctgggagtcctggtcacataggacccccaggccccactggacccaaaggaatctctggccacccaggagagaagggcgagagaggactgcgtggggagcctggcccccaaggctctgctgggcagcggggggaacctggccctaagggagaccctggtgagaagagccactgggctcctagcttacagagcttcctgcagcagcaggctcagctggagctcctggccagacgggtcaccctcctggaagccatcatctggccagaaccagagctggggtctggggcgggccctgccggcacaggcacccccagcctccttcggggcaagaggggcggacatgcaaccaactaccggatcgtggcccccaggagccgggacgagagaggctgagggtggtggcggcccctgaggcagaccaggccaggcttcccctcctacctggactcggccagctgcctccagggaccgcccgtccatatttattaatgtcctcagggtcccttctgccatctaggccttaggggtaagcaggtctcagtcctggcaccatgcacatgtctgaggctgagcaagggctgagaggagaggcttgggcctcagtttccctctgtgaagtggggggaggcaggccttcaaggagggatagaggtacaaggcttcgtctcatctgctgtctgagcatccaggcccaaaggcactgagggagtcaggagctggggctcggcacatgcagagatgacagggcagggggcagtcttcctccccctccccgaccaaacctcggggagccctcctgtgcccctccctccttgttgtccagtgctgggctccccaccccgaggtcaggctgcccaatcctctgactggatcaccgggggcttcttgcctcagttcttccctctgagcccccaggccctcccgcatctcaggttggggatggggacatggagaggaaggggccgcctactcctgcaaatgcttgtgacagatgccaggaggtagatgtgtgctggccaataaaggcccctacctgattccccgca

SEQ ID NO: 64 - DNA, 2060 bp, Homo sapiens
cgggcaggcaggcggggaggacaggctgggggcggcgaccgcgaggggccgcgcgcggagggcgcctggtgcagcatgggcggcccgcgggcttgggcgctgctctgcctcgggctcctgctcccgggaggcggcgctgcgtggagcatcggggcagctccgttctccggacgcaggaactggtgctcctatgtggtgacccgcaccatctcatgccatgtgcagaatggcacctaccttcagcgagtgctgcagaactgcccctggcccatgagctgtccggggagcagctacagaactgtggtgagacccacatacaaggtgatgtacaagatagtgaccgcccgtgagtggaggtgctgccctgggcactcaggagtgagctgcgaggaagttgcagcttcctctgcctccttggagcccatgtggtcgggcagtaccatgcggcggatggcgcttcggcccacagccttctcaggttgtctcaactgcagcaaagtgtcagagctgacagagcggctgaaggtgctggaggccaagatgaccatgctgactgtcatagagcagccagtacctccaacaccagctacccctgaggaccctgccccgctctggggtccccctcctgcccagggcagccccggagatggaggcctccaggaccaagtcggtgcttgggggcttcccgggcccaccggccccaagggagatgccggcagtcggggcccaatggggatgagaggcccaccaggtccacagggccccccagggagccctggccgggctggagctgtgggcacccctggagagaggggacctcctgggccaccagggcctcctggcccccctgggcccccagcccctgttgggccaccccatgcccggatctcccagcatggagacccattgctgtccaacaccttcactgagaccaacaaccactggccccagggacccactgggcctccaggccctccagggcccatgggtccccctgggcctcctggccccacaggtgtccctgggagtcctggtcacataggacccccaggccccactggacccaaaggaatctctggccacccaggagagaagggcgagagaggactgcgtggggagcctggcccccaaggctctgctgggcagcggggggaacctggccctaagggagaccctggtgagaagagccactggggggaggggttgcaccagctacgcgaggctttgaagattttagctgagagggttttaatcttggaaacaatgattgggctctatgaaccagagctggggtctggggcgggccctgccggcacaggcacccccagcctccttcggggcaagaggggcggacatgcaaccaactaccggatcgtggcccccaggagccgggacgagagaggctgagggtggtggcggcccctgaggcagaccaggccaggcttcccctcctacctggactcggccagctgcctccagggaccgcccgtccatatttattaatgtcctcagggtcccttctgccatctaggccttaggggtaagcaggtctcagtcctggcaccatgcacatgtctgaggctgagcaagggctgagaggagaggcttgggcctcagtttccctctgtgaagtggggggaggcaggccttcaaggagggatagaggtacaaggcttcgtctcatctgctgtctgagcatccaggcccaaaggcactgagggagtcaggagctggggctcggcacatgcagagatgacagggcagggggcagtcttcctccccctccccgaccaaacctcggggagccctcctgtgcccctccctccttgttgtccagtgctgggctccccaccccgaggtcaggctgcccaatcctctgactggatcaccgggggcttcttgcctcagttcttccctctgagcccccaggccctcccgcatctcaggttggggatggggacatggagaggaaggggccgcctactcctgcaaatgcttgtgacagatgccaggaggtagatgtgtgctggccaataaaggcccctacctgattccccgca

SEQ ID NO: 65 - DNA, 2054 bp, Homo sapiens
cgggcaggcaggcggggaggacaggctgggggcggcgaccgcgaggggccgcgcgcggagggcgcctggtgcagcatgggcggcccgcgggcttgggcgctgctctgcctcgggctcctgctcccgggaggcggcgctgcgtggagcatcggggcagctccgttctccggacgcaggaactggtgctcctatgtggtgacccgcaccatctcatgccatgtgcagaatggcacctaccttcagcgagtgctgcagaactgcccctggcccatgagctgtccggggagcagctacagaactgtggtgagacccacatacaaggtgatgtacaagatagtgaccgcccgtgagtggaggtgctgccctgggcactcaggagtgagctgcgaggaagcttcctctgcctccttggagcccatgtggtcgggcagtaccatgcggcggatggcgcttcggcccacagccttctcaggttgtctcaactgcagcaaagtgtcagagctgacagagcggctgaaggtgctggaggccaagatgaccatgctgactgtcatagagcagccagtacctccaacaccagctacccctgaggaccctgccccgctctggggtccccctcctgcccagggcagccccggagatggaggcctccaggaccaagtcggtgcttgggggcttcccgggcccaccggccccaagggagatgccggcagtcggggcccaatggggatgagaggcccaccaggtccacagggccccccagggagccctggccgggctggagctgtgggcacccctggagagaggggacctcctgggccaccagggcctcctggcccccctgggcccccagcccctgttgggccaccccatgcccggatctcccagcatggagacccattgctgtccaacaccttcactgagaccaacaaccactggccccagggacccactgggcctccaggccctccagggcccatgggtccccctgggcctcctggccccacaggtgtccctgggagtcctggtcacataggacccccaggccccactggacccaaaggaatctctggccacccaggagagaagggcgagagaggactgcgtggggagcctggcccccaaggctctgctgggcagcggggggaacctggccctaagggagaccctggtgagaagagccactggggggaggggttgcaccagctacgcgaggctttgaagattttagctgagagggttttaatcttggaaacaatgattgggctctatgaaccagagctggggtctggggcgggccctgccggcacaggcacccccagcctccttcggggcaagaggggcggacatgcaaccaactaccggatcgtggcccccaggagccgggacgagagaggctgagggtggtggcggcccctgaggcagaccaggccaggcttcccctcctacctggactcggccagctgcctccagggaccgcccgtccatatttattaatgtcctcagggtcccttctgccatctaggccttaggggtaagcaggtctcagtcctggcaccatgcacatgtctgaggctgagcaagggctgagaggagaggcttgggcctcagtttccctctgtgaagtggggggaggcaggccttcaaggagggatagaggtacaaggcttcgtctcatctgctgtctgagcatccaggcccaaaggcactgagggagtcaggagctggggctcggcacatgcagagatgacagggcagggggcagtcttcctccccctccccgaccaaacctcggggagccctcctgtgcccctccctccttgttgtccagtgctgggctccccaccccgaggtcaggctgcccaatcctctgactggatcaccgggggcttcttgcctcagttcttccctctgagcccccaggccctcccgcatctcaggttggggatggggacatggagaggaaggggccgcctactcctgcaaatgcttgtgacagatgccaggaggtagatgtgtgctggccaataaaggcccctacctgattccccgca

SEQ ID NO: 66 - DNA, 2048 bp, Homo sapiens
cgggcaggcaggcggggaggacaggctgggggcggcgaccgcgaggggccgcgcgcggagggcgcctggtgcagcatgggcggcccgcgggcttgggcgctgctctgcctcgggctcctgctcccgggaggcggcgctgcgtggagcatcggggcagctccgttctccggacgcaggaactggtgctcctatgtggtgacccgcaccatctcatgccatgtgcagaatggcacctaccttcagcgagtgctgcagaactgcccctggcccatgagctgtccggggagcagaactgtggtgagacccacatacaaggtgatgtacaagatagtgaccgcccgtgagtggaggtgctgccctgggcactcaggagtgagctgcgaggaagcttcctctgcctccttggagcccatgtggtcgggcagtaccatgcggcggatggcgcttcggcccacagccttctcaggttgtctcaactgcagcaaagtgtcagagctgacagagcggctgaaggtgctggaggccaagatgaccatgctgactgtcatagagcagccagtacctccaacaccagctacccctgaggaccctgccccgctctggggtccccctcctgcccagggcagccccggagatggaggcctccaggaccaagtcggtgcttgggggcttcccgggcccaccggccccaagggagatgccggcagtcggggcccaatggggatgagaggcccaccaggtccacagggccccccagggagccctggccgggctggagctgtgggcacccctggagagaggggacctcctgggccaccagggcctcctggcccccctgggcccccagcccctgttgggccaccccatgcccggatctcccagcatggagacccattgctgtccaacaccttcactgagaccaacaaccactggccccagggacccactgggcctccaggccctccagggcccatgggtccccctgggcctcctggccccacaggtgtccctgggagtcctggtcacataggacccccaggccccactggacccaaaggaatctctggccacccaggagagaagggcgagagaggactgcgtggggagcctggcccccaaggctctgctgggcagcggggggaacctggccctaagggagaccctggtgagaagagccactggggggaggggttgcaccagctacgcgaggctttgaagattttagctgagagggttttaatcttggaaacaatgattgggctctatgaaccagagctggggtctggggcgggccctgccggcacaggcacccccagcctccttcggggcaagaggggcggacatgcaaccaactaccggatcgtggcccccaggagccgggacgagagaggctgagggtggtggcggcccctgaggcagaccaggccaggcttcccctcctacctggactcggccagctgcctccagggaccgcccgtccatatttattaatgtcctcagggtcccttctgccatctaggccttaggggtaagcaggtctcagtcctggcaccatgcacatgtctgaggctgagcaagggctgagaggagaggcttgggcctcagtttccctctgtgaagtggggggaggcaggccttcaaggagggatagaggtacaaggcttcgtctcatctgctgtctgagcatccaggcccaaaggcactgagggagtcaggagctggggctcggcacatgcagagatgacagggcagggggcagtcttcctccccctccccgaccaaacctcggggagccctcctgtgcccctccctccttgttgtccagtgctgggctccccaccccgaggtcaggctgcccaatcctctgactggatcaccgggggcttcttgcctcagttcttccctctgagcccccaggccctcccgcatctcaggttggggatggggacatggagaggaaggggccgcctactcctgcaaatgcttgtgacagatgccaggaggtagatgtgtgctggccaataaaggcccctacctgattccccgca

SEQ ID NO: 67 - DNA, 1997 bp, Homo sapiens
cgggcaggcaggcggggaggacaggctgggggcggcgaccgcgaggggccgcgcgcggagggcgcctggtgcagcatgggcggcccgcgggcttgggcgctgctctgcctcgggctcctgctcccgggaggcggcgctgcgtggagcatcggggcagctccgttctccggacgcaggaactggtgctcctatgtggtgacccgcaccatctcatgccatgtgcagaatggcacctaccttcagcgagtgctgcagaactgcccctggcccatgagctgtccggggagcagctacagaactgtggtgagacccacatacaaggtgatgtacaagatagtgaccgcccgtgagtggaggtgctgccctgggcactcaggagtgagctgcgaggaagttgcagcttcctctgcctccttggagcccatgtggtcgggcagtaccatgcggcggatggcgcttcggcccacagccttctcaggttgtctcaactgcagcaaagtgtcagagctgacagagcggctgaaggtgctggaggccaagatgaccatgctgactgtcatagagcagccagtacctccaacaccagctacccctgaggaccctgccccgctctggggtccccctcctgcccagggcagccccggagatggaggcctccaggaccaagtcggtgcttgggggcttcccgggcccaccggccccaagggagatgccggcagtcggggcccaatggggatgagaggcccaccaggtccacagggccccccagggagccctggccgggctggagctgtgggcacccctggagagaggggacctcctgggccaccagggcctcctggcccccctgggcccccagcccctgttgggccaccccatgcccggatctcccagcatggagacccattgctgtccaacaccttcactgagaccaacaaccactggccccagggacccactgggcctccaggccctccagggcccatgggtccccctgggcctcctggccccacaggtgtccctgggagtcctggtcacataggactgcgtggggagcctggcccccaaggctctgctgggcagcggggggaacctggccctaagggagaccctggtgagaagagccactggggggaggggttgcaccagctacgcgaggctttgaagattttagctgagagggttttaatcttggaaacaatgattgggctctatgaaccagagctggggtctggggcgggccctgccggcacaggcacccccagcctccttcggggcaagaggggcggacatgcaaccaactaccggatcgtggcccccaggagccgggacgagagaggctgagggtggtggcggcccctgaggcagaccaggccaggcttcccctcctacctggactcggccagctgcctccagggaccgcccgtccatatttattaatgtcctcagggtcccttctgccatctaggccttaggggtaagcaggtctcagtcctggcaccatgcacatgtctgaggctgagcaagggctgagaggagaggcttgggcctcagtttccctctgtgaagtggggggaggcaggccttcaaggagggatagaggtacaaggcttcgtctcatctgctgtctgagcatccaggcccaaaggcactgagggagtcaggagctggggctcggcacatgcagagatgacagggcagggggcagtcttcctccccctccccgaccaaacctcggggagccctcctgtgcccctccctccttgttgtccagtgctgggctccccaccccgaggtcaggctgcccaatcctctgactggatcaccgggggcttcttgcctcagttcttccctctgagcccccaggccctcccgcatctcaggttggggatggggacatggagaggaaggggccgcctactcctgcaaatgcttgtgacagatgccaggaggtagatgtgtgctggccaataaaggcccctacctgattccccgca

SEQ ID NO: 68 - DNA, 1823 bp, Homo sapiens
cgggcaggcaggcggggaggacaggctgggggcggcgaccgcgaggggccgcgcgcggagggcgcctggtgcagcatgggcggcccgcgggcttgggcgctgctctgcctcgggctcctgctcccgggaggcggcgctgcgtggagcatcggggcagctccgttctccggacgcaggaactggtgctcctatgtggtgacccgcaccatctcatgccatgtgcagaatggcacctaccttcagcgagtgctgcagaactgcccctggcccatgagctgtccggggagcagctacagaactgtggtgagacccacatacaaggtgatgtacaagatagtgaccgcccgtgagtggaggtgctgccctgggcactcaggagtgagctgcgaggaagttgcagcttcctctgcctccttggagcccatgtggtcgggcagtaccatgcggcggatggcgcttcggcccacagccttctcaggttgtctcaactgcagcaaagtgtcagagctgacagagcggctgaaggtgctggaggccaagatgaccatgctgactgtcatagagcagccagtacctccaacaccagctacccctgaggaccctgccccgctctggggtccccctcctgcccagggcagccccggagatggaggcctccagggagacccattgctgtccaacaccttcactgagaccaacaaccactggccccagggacccactgggcctccaggccctccagggcccatgggtccccctgggcctcctggccccacaggtgtccctgggagtcctggtcacataggacccccaggccccactggacccaaaggaatctctggccacccaggagagaagggcgagagaggactgcgtggggagcctggcccccaaggctctgctgggcagcggggggaacctggccctaagggagaccctggtgagaagagccactggggggaggggttgcaccagctacgcgaggctttgaagattttagctgagagggttttaatcttggaaacaatgattgggctctatgaaccagagctggggtctggggcgggccctgccggcacaggcacccccagcctccttcggggcaagaggggcggacatgcaaccaactaccggatcgtggcccccaggagccgggacgagagaggctgagggtggtggcggcccctgaggcagaccaggccaggcttcccctcctacctggactcggccagctgcctccagggaccgcccgtccatatttattaatgtcctcagggtcccttctgccatctaggccttaggggtaagcaggtctcagtcctggcaccatgcacatgtctgaggctgagcaagggctgagaggagaggcttgggcctcagtttccctctgtgaagtggggggaggcaggccttcaaggagggatagaggtacaaggcttcgtctcatctgctgtctgagcatccaggcccaaaggcactgagggagtcaggagctggggctcggcacatgcagagatgacagggcagggggcagtcttcctccccctccccgaccaaacctcggggagccctcctgtgcccctccctccttgttgtccagtgctgggctccccaccccgaggtcaggctgcccaatcctctgactggatcaccgggggcttcttgcctcagttcttccctctgagcccccaggccctcccgcatctcaggttggggatggggacatggagaggaaggggccgcctactcctgcaaatgcttgtgacagatgccaggaggtagatgtgtgctggccaataaaggcccctacctgattccccgca

SEQ ID NO: 69 - DNA, 1976 bp, Homo sapiens
cgggcaggcaggcggggaggacaggctgggggcggcgaccgcgaggggccgcgcgcggagggcgcctggtgcagcatgggcggcccgcgggcttgggcgctgctctgcctcgggctcctgctcccgggaggcggcgctgcgtggagcatcggggcagctccgttctccggacgcaggaactggtgctcctatgtggtgacccgcaccatctcatgccatgtgcagaatggcacctaccttcagcgagtgctgcagaactgcccctggcccatgagctgtccggggagcagctacagaactgtggtgagacccacatacaaggtgatgtacaagatagtgaccgcccgtgagtggaggtgctgccctgggcactcaggagtgagctgcgaggaaggttgtctcaactgcagcaaagtgtcagagctgacagagcggctgaaggtgctggaggccaagatgaccatgctgactgtcatagagcagccagtacctccaacaccagctacccctgaggaccctgccccgctctggggtccccctcctgcccagggcagccccggagatggaggcctccaggaccaagtcggtgcttgggggcttcccgggcccaccggccccaagggagatgccggcagtcggggcccaatggggatgagaggcccaccaggtccacagggccccccagggagccctggccgggctggagctgtgggcacccctggagagaggggacctcctgggccaccagggcctcctggcccccctgggcccccagcccctgttgggccaccccatgcccggatctcccagcatggagacccattgctgtccaacaccttcactgagaccaacaaccactggccccagggacccactgggcctccaggccctccagggcccatgggtccccctgggcctcctggccccacaggtgtccctgggagtcctggtcacataggacccccaggccccactggacccaaaggaatctctggccacccaggagagaagggcgagagaggactgcgtggggagcctggcccccaaggctctgctgggcagcggggggaacctggccctaagggagaccctggtgagaagagccactggggggaggggttgcaccagctacgcgaggctttgaagattttagctgagagggttttaatcttggaaacaatgattgggctctatgaaccagagctggggtctggggcgggccctgccggcacaggcacccccagcctccttcggggcaagaggggcggacatgcaaccaactaccggatcgtggcccccaggagccgggacgagagaggctgagggtggtggcggcccctgaggcagaccaggccaggcttcccctcctacctggactcggccagctgcctccagggaccgcccgtccatatttattaatgtcctcagggtcccttctgccatctaggccttaggggtaagcaggtctcagtcctggcaccatgcacatgtctgaggctgagcaagggctgagaggagaggcttgggcctcagtttccctctgtgaagtggggggaggcaggccttcaaggagggatagaggtacaaggcttcgtctcatctgctgtctgagcatccaggcccaaaggcactgagggagtcaggagctggggctcggcacatgcagagatgacagggcagggggcagtcttcctccccctccccgaccaaacctcggggagccctcctgtgcccctccctccttgttgtccagtgctgggctccccaccccgaggtcaggctgcccaatcctctgactggatcaccgggggcttcttgcctcagttcttccctctgagcccccaggccctcccgcatctcaggttggggatggggacatggagaggaaggggccgcctactcctgcaaatgcttgtgacagatgccaggaggtagatgtgtgctggccaataaaggcccctacctgattccccgca

SEQ ID NO: 70 - DNA, 2111 bp, Homo sapiens
cgggcaggcaggcggggaggacaggctgggggcggcgaccgcgaggggccgcgcgcggagggcgcctggtgcagcatgggcggcccgcgggcttgggcgctgctctgcctcgggctcctgctcccgggaggcggcgctgcgtggagcatcggggcagctccgttctccggacgcaggaactggtgctcctatgtggtgacccgcaccatctcatgccatgtgcagaatggcacctaccttcagcgagtgctgcagaactgcccctggcccatgagctgtccggggagcagctacagaactgtggtgagacccacatacaaggtgatgtacaagatagtgaccgcccgtgagtggaggtgctgccctgggcactcaggagtgagctgcgaggaagttgcagcttcctctgcctccttggagcccatgtggtcgggcagtaccatgcggcggatggcgcttcggcccacagccttctcaggttgtctcaactgcagcaaagtgtcagagctgacagagcggctgaaggtgctggaggccaagatgaccatgctgactgtcatagagcagccagtacctccaacaccagctacccctgaggaccctgccccgctctggggtccccctcctgcccagggcagccccggagatggaggcctccaggggctgccaggagccatagagagtgtgagggtcccgctgcttccccgaaatgaccaagtcggtgcttgggggcttcccgggcccaccggccccaagggagatgccggcagtcggggcccaatggggatgagaggcccaccaggtccacagggccccccagggagccctggccgggctggagctgtgggcacccctggagagaggggacctcctgggccaccagggcctcctggcccccctgggcccccagcccctgttgggccaccccatgcccggatctcccagcatggagacccattgctgtccaacaccttcactgagaccaacaaccactggccccagggacccactgggcctccaggccctccagggcccatgggtccccctgggcctcctggccccacaggtgtccctgggagtcctggtcacataggacccccaggccccactggacccaaaggaatctctggccacccaggagagaagggcgagagaggactgcgtggggagcctggcccccaaggctctgctgggcagcggggggaacctggccctaagggagaccctggtgagaagagccactggggggaggggttgcaccagctacgcgaggctttgaagattttagctgagagggttttaatcttggaaacaatgattgggctctatgaaccagagctggggtctggggcgggccctgccggcacaggcacccccagcctccttcggggcaagaggggcggacatgcaaccaactaccggatcgtggcccccaggagccgggacgagagaggctgagggtggtggcggcccctgaggcagaccaggccaggcttcccctcctacctggactcggccagctgcctccagggaccgcccgtccatatttattaatgtcctcagggtcccttctgccatctaggccttaggggtaagcaggtctcagtcctggcaccatgcacatgtctgaggctgagcaagggctgagaggagaggcttgggcctcagtttccctctgtgaagtggggggaggcaggccttcaaggagggatagaggtacaaggcttcgtctcatctgctgtctgagcatccaggcccaaaggcactgagggagtcaggagctggggctcggcacatgcagagatgacagggcagggggcagtcttcctccccctccccgaccaaacctcggggagccctcctgtgcccctccctccttgttgtccagtgctgggctccccaccccgaggtcaggctgcccaatcctctgactggatcaccgggggcttcttgcctcagttcttccctctgagcccccaggccctcccgcatctcaggttggggatggggacatggagaggaaggggccgcctactcctgcaaatgcttgtgacagatgccaggaggtagatgtgtgctggccaataaaggcccctacctgattccccgca

SEQ ID NO: 71 - DNA, 707 bp, Homo sapiens
agatgaccatgctgactgtcatagagcagccagtacctccaacaccagctacccctgaggaccctgccccgctctggggtccccctcctgcccagggcagccccggagatggaggcctccaggggctgccaggagccatagagagtgtgagggtcccgctgcttccccgaaatgaccaagtcggtgcttgggggcttcccgggcccaccggccccaagggagatgccggcagtcggggcccaatggggatgagaggcccaccaggtccacagggccccccagggagccctggccgggctggagctgtgggcacccctggagagaggggacctcctgggccaccagggcctcctggcccccctgggcccccagcccctgttgggccaccccatgcccggatctcccagcatggagacccattgctgtccaacaccttcactgagaccaacaaccactggccccagggacccactgggcctccaggccctccagggcccatgggtccccctgggcctcctggccccacaggtgtccctgggagtcctggtcacataggacccccaggccccactggacccaaaggaatctctggccacccaggagagaagggcgagagaggactgcgtggggagcctggcccccaaggctctgctgggcagcggggggaacctggccctaagggagaccctggtgagaagagccactgg

SEQ ID NO: 72 - DNA, 2034 bp, Homo sapiens
ggtgagtgcccgcaatgctgccccacagctcctctggccatcccctccaccaggtgggcccttccctgctcctgacatggccaggatgacctgggccctttcatctacttgcctcttcactcagcaccccaccacggagtgccctgcccacgcctgggctccatgaagtcctctcttatgttcactgacccacattccctgggcacctacacttatcaggctctgagctgggcactgggtggggtcagacatgtccctgcccttctggagcttccatgctgctgggagcagggctgggcagaggagaagcagcaatgcttgccccatgtgaccagggtttctatgaggggttttgggggtttgggagccccaaggaaggaagactcagcctggacgaggtggagaactaggtgctgtgctcatccccctgttagactaccaggcagcctaggcctgtggactccggggccctctctcatgcccactgctccaggctgccttgtcctgtcgctcaaggccaccctgggcctccttgcctcctgtatacccacaaatccgtgtgattccattgcaggtccacagggccccccagggagccctggccgggctggagctgtgggcacccctggagagaggggacctcctgggccaccagggcctcctggcccccctgggcccccagcccctgttgggccaccccatgcccggatctcccagcatggtgagtccccctgggatcccagcaggtggaggtgggggtggagtagccatcagcacagtgcccgctaccatctgccacgtgccttctgtgtgccagccctgctcacgataggccacatgtgacccagtcctccagcaggcgccgttgtcctcctgtggttacaggtgaggaacactgaggaccagagagggaaggtggcttgccagggtcccacagcctgggcgtaggggaacggcttcaaacccaggctgcctccagaacctgtgcttagagccaccgggcatcaggccctcccaagccttggaactggctggaatccagttctcggaacactgggacgcaaaagacccggcggcaggaagtgagtcctgaactcccaaggccacaggcccggcccctcctccaggccctgacgtgcgtccttggcttcttccctttggcagcccagcctgacctgcccatgggctgccaggggtcagagtgtggagcgccaggtttcagcctcttctccactgtgtttttggtgcacaacccagcacaccattcattcattctgccatcccagcattcattccatctcactatccatacgatggggacaatgacagtgccagcctcccagagctgcgtaacatccatgtacagaagcctggcacacagtaggtggtggataaatggtatcttttattgtcattcccatttgacaggtgacagtacaggctctgaaaagtagaaagtgttgctggatgtcaccagctggattgcagtggggttagaacccacatctccctgcctcctggtcttgcgggaccaacactctccacactcctcaccctggagcaggtgcccaggtggtaccagccatgctgcaggctgccccatagggcagtccaagctgtcttggcagaggtggcaggtgaagactaaccaccccactctacccagctctactcactcatcatctttgctcacccaggagacccattgctgtccaacaccttcactgagaccaacaaccactggccccagggacccactgggcctccaggccctccagggcccatgggtccccctgggcctcctggccccacaggtgtccctgggagtcctggtcacatagtgagtagttctccttgtactctcacccatgtgtctgtccatctttccatctatgcatacatccatacatctgtccatcatccacccttgtatccatctatccatccatccattcatccttccattcattcattcaacaagtatttattgagcacttaatatgcaaactaccttccataaatcttattcaatcc

SEQ ID NO: 73 - protein, 518 aa, Homo sapiens
MLAAASKYRHGNMVFFDVLGLFVIAYPSRIGSIINYMVVMGVVLYLGKKFLQPKHKTGNYKKDFLCGLGITLISWFTSLVTVLIIAVFISLIGQSLSWYNHFYVSVCLYGTATVAKIILIHTLAKRFYYMNASAQYLGEVFFDISLFVHCCFLVTLTYQGLCSAFISAVWVAFPLLTKLCVHKDFKQHGAQGKFIAFYLLGMFIPYLYALYLIWAVFEMFTPILGRSGSEIPPDVVLASILAGCTMILSSYFINFIYLAKSTKKTMLTLTLVCAITFLLVCSGTFFPYSSNPANPKPKRVFLQHMTRTFHDLEGNAVKRDSGIWINGFDYTGISHITPHIPEINDSIRAHCEENAPLCGFPWYLPVHFLIRKNWYLPAPEVSPRNPPHFRLISKEQTPWDSIKLTFEATGPSHMSFYVRAHKGSTLSQWSLGNGTPVTSKGGDYFVFYSHGLQASAWQFWIEVQVSEEHPEGMVTVAIAAHYLSGEDKRSPQLDALKEKFPDWTFPSAWVCTYDLFVF

SEQ ID NO: 74 - protein, 904 aa, Homo sapiens
MEWGSESAAVRRHRVGVERREGAAAAPPPEREARAQEPLVDGCSGGGRTRKRSPGGSGGASRGAGTGLSEVRAALGLALYLIALRTLVQLSLQQLVLRGAAGHRGEFDALQARDYLEHITSIGPRTTGSPENEILTVHYLLEQIKLIEVQSNSLHKISVDVQRPTGSFSIDFLGGFTSYYDNITNVVVKLEPRDGAQHAVLANCHFDSVANSPGASDDAVSCSVMLEVLRVLSTSSEALHHAVIFLFNGAEENVLQASHGFITQHPWASLIRAFINLEAAGVGGKELVFQTGPENPWLVQAYVSAAKHPFASVVAQEVFQSGIIPSDTDFRIYRDFGNIPGIDLAFIENGYIYHTKYDTADRILTDSIQRAGDNILAVLKHLATSDMLAAASKYRHGNMVFFDVLGLFVIAYPSRIGSIINYMVVMGVVLYLGKKFLQPKHKTGNYKKDFLCGLGITLISWFTSLVTVLIIAVFISLIGQSLSWYNHFYVSVCLYGTATVAKIILIHTLAKRFYYMNASAQYLGEVFFDISLFVHCCFLVTLTYQGLCSAFISAVWVAFPLLTKLCVHKDFKQHGAQGKFIAFYLLGMFIPYLYALYLIWAVFEMFTPILGRSGSEIPPDVVLASILAGCTMILSSYFINFIYLAKSTKKTMLTLTLVCAITFLLVCSGTFFPYSSNPANPKPKRVFLQHMTRTFHDLEGNAVKRDSGIWINGFDYTGISHITPHIPEINDSIRAHCEENAPLCGFPWYLPVHFLIRKNWYLPAPEVSPRNPPHFRLISKEQTPWDSIKLTFEATGPSHMSFYVRAHKGSTLSQWSLGNGTPVTSKGGDYFVFYSHGLQASAWQFWIEVQVSEEHPEGMVTVAIAAHYLSGEDKRSPQLDALKEKFPDWTFPSAWVCTYDLFVF

SEQ ID NO: 75 - protein, 419 aa, Homo sapiens
MVVMGVVLYLGKKFLQPKHKTGNYKKDFLCGLGITLISWFTSLVTVLIIAVFISLIGQSLSWYNHFYVSVCLYGTATVAKIILIHTLAKRFYYMNASAQYLGEVFFDISLFVHCCFLVTLTYQGLCSAFISAVWVAFPLLTKLCVHKDFKQHGAQGKFIAFYLLGMFIPYLYALYLIWAVFEMFTPILGRSGSEIPPDVVLASILAGCTMILSSYFINFIYLAKSTKKTMLTLTLVCAITFLLVCSGTFFPYSSNPANPKPKRVFLQHMTRTFHDLEGNAVKRDSGIWINGFDYTGISHITPHIPEINDSIRAHCEENAPLCGFPWYLPVHFLIRKNWYLPAPEVSPRNPPHFRLISKEQTPWDSIKLTFEATACLPILQILDLPASTIMTKPYVLLCSSPQRVNTFSVVSWQWHPSHK

SEQ ID NO: 76 - DNA, 4974 bp, Homo sapiens
ggcgcggggaccgggctgtctgaggtgcgcgccgcgctggggctcgcgctctacctgatcgcgctgcggacgctggtgcagctctcgctgcagcagctcgtgctacgcggggccgctggacaccgcggggagttcgacgcgctccaagccagggattatcttgaacacataacctccattggccccaggactacaggaagtccagaaaatgaaattctgaccgtgcactaccttttggaacagattaaactgattgaagtgcaaagcaacagccttcataagatttcagtagatgtacaacggcccacaggctcttttagcattgatttcttgggaggttttacaagctattatgacaacatcaccaatgttgtggtaaagctggaacccagagatggagcccagcatgctgtcttggctaattgtcattttgactcagtagcaaactcaccaggccagtcatggtttcattactcagcacccctgggctagcttgattcgtgcattcattaacctagaggcagcaggtgtaggagggaaagaacttgtattccaaacaggtcctgaaaatccttggttggttcaagcttatgtttcagcagctaaacacccttttgcttctgtggtggctcaggaggtttttcagagtggaatcattccttcagatactgactttcgtatctacagggattttgggaacattccaggaatagacttagcttttattgagaatggatacatttatcacaccaagtatgacacagcggacagaattctaacagattccattcagagagcaggtgacaacattttagcagttcttaagcatctagctacatctgatatgctggctgctgcttctaagtatcgacatggaaacatggtcttctttgatgtgctgggcctgtttgtcattgcctacccctctcgtattggctcaatcataaactacatggtggtaatgggtgttgttttgtacctgggcaaaaaatttttgcagcccaaacataagactggtaactacaagaaggacttcttgtgtggacttggcatcactttgatcagctggttcactagccttgttaccgttctcattatagcagtgttcatctctcttattggacagtctctctcatggtataaccacttctatgtctccgtttgtctgtatggaactgcaactgtagccaaaataatacttatacatactcttgcgaaaagattttattacatgaatgccagtgcccagtatctgggagaagtattttttgacatttcgctgtttgtccattgctgttttcttgttaccctcacttaccaaggactttgctcggcgtttattagtgctgtctgggtagcattcccattgctcacaaagctctgtgtgcataaggacttcaagcagcatggtgcccaaggaaaatttattgctttttaccttttggggatgtttattccttatctttatgcattgtacctcatctgggcagtatttgagatgtttacccctatcctcgggagaagtggttctgaaatcccacctgatgttgtgctggcatccattttggctggctgtacaatgattctctcgtcctattttattaacttcatctaccttgccaagagcacaaaaaaaaccatgctaactttaactttggtatgtgcaattacattcctccttgtttgcagtggaacattttttccatatagctccaatcctgctaatccgaagccaaagagagtgtttcttcagcatatgactagaacattccatgacttggaaggaaatgcagttaaacgggactctggaatatggatcaatgggtttgattatactggaatttctcacataacccctcacattcctgagatcaatgatagtatccgagctcactgtgaggagaatgcacctctttgtggttttccttggtatcttccagtgcactttctgatcaggaaaaactggtatcttcctgccccagaagtttctccaagaaatcctcctcatttccgactcatatccaaagaacagacaccttgggattctataaaattgacttttgaagcaacaggaccaagccatatgtccttctatgttcgagcccacaaagggtcaacactttctcagtggtctcttggcaatggcaccccagtcacaagtaaaggaggagactactttgtcttttactcccatggactccaggcctctgcatggcagttctggatagaagtgcaggtttcagaagaacatcctgaaggaatggtcaccgtggccattgctgcccactatctgtctggggaagacaagagatcccctcaactggatgctctgaaggaaaagttcccagattggacatttccctctgcctgggtgtgcacctacgatctctttgtattttaatcttgtggatgagctctaagtacatgcccagtggatactccatgtgacatggtttctccctatgttacgtggatgtttgtaacgtaagtcaatgaattttaatgatcatatgttcaaagagctttctgggttaacgcttttcagggccaagcactataagggtttagctgtggcgcagtgatgcatggcctgttgacacttgaaaatgccagtcttttggcacttcagcacatgtgggtactgccactacacacacgtcattttatatgaccttaaggacaaagccaacaatccacttcaatagctgcccctttaggatcaagaaagatgtacactgtcagagcattgttaatgagacaaaagttgtttccaatttaagccccaaaaccatttgttgtattagtggatggtgggtaaaatatcattcactgaggtaatgattccccttgagaatataactctgtgtaggtcactggaaagtgattgccatagggctgggagagaagcattgcactcttgaggctgtagcctgtgtcaagctgtttcttcaggcagcctctcaaatgtgctttgtctctctgtgctgaggcctggaccctgtgctgagctggtgactcactgtcctgacaagtggacacacagatgcactgctgtgctgctttcctgaggtggttttctatgcctgttttcctctgaaacatgtctgttacccctctccatcttaccaagttgaaaaggggaatatttggccacatacccctctggttttcgtaggttcttttggttcagaatattgtttgtgccagtacatgaccttaacttccttcctcagagcactgagctgccatctgggctattctggggtagaaggaaggctgggagtggtgggaattttataaatatttattctcttttctttgtttcataggagtcttgtgttatacaaggttagtccttcatggtataatcttactgatgcactgggcctatctttttgttttccagccagttgaatagattagtttttctcagtaacttactatccagcagactggctttcctgagacttgaggttgtggcttatactggaatgagaccactgtacgtgtaggtggttcagatcctgcgtaatggcagcatgaggacttaaaaggtggttttcattttgaagatggctatgtagcttgtaaggtgtatcacagcagtacctctcatggctttttggttccagcagtgagggcattggtgagatcaatggtaaactgtgcaagctttctttttatcattaggaaatgtgaaacgttggacaaattttgagttttaacaaggacaaaaagttgaaagaaaaggcacagttaacaaaaaagggtggctagatttatcttgggtgatggaggaaatgagagaggaatgctcttgaaaggtggtctgtggatctgtctgaatagaaagagcacagtaagtatgcattgccggagaaaacgtccttgaagctgcttgtctcatgtgtatgatgtgctttttaaatcatgcccctcgttgcctgcctaatctgtgactccctaaaaactaactgggcccatgtagatggggctgcaaccagagctgaataacatgttaggctcacacatgcatcagcactgcacactggaatcattgctcttcctggactttgtagaaatcagtctcaagtgcttcaagagtctggctcctgctacttttatctgtcaggtagcacataaggtttgcagggtttatattttgtatagaatcacagttgtggagaaaaagtaataatttctcaatgaattttaaaaatgggcctattttctatccccgtggttcatctgatataattagtgttccctgtgaattccccccctctatgggaaggatgcctttactctttatcagtaataaattatgactgttttcatattgccttagggttatttccctgtgtaaaccattgtcttttgttttggttttctttagcattatgaagctttggtattgtacaaggtcagtagtaagatgctcactagtctcagggcttgtgtaatattctgggaggtcatttaaatgccagaaatggtcaagcaattatacacagtatttatgactctgttaagcataccgtttgtctgtcacattagtagattctgagattaaaaaaaatttttaaagagtgatcatttaaataatttctaaaagggtcttttcaagctctaacaaagtcactaacaaatgcattattttctacagaattagatgttagtagtacagtactgcatattcagggaaaaagtgtgaggaattgatttcaaaatagttcgttcttgtgtttgacctaagaatgattgtcgcatgaagtgtttgtttttacagtttagcatatataaacaaacatgataggattccttaagatgttaccacccagggggccacaagccagcctgctgtctcaggaagctgtagaaggagtgtttgtcaatttcttgtcactggtttgctgacttactgaggattaattgttgccttacaatgttactgaaataaactgtttaatatac

SEQ ID NO: 77 - DNA, 5338 bp, Homo sapiens
ggccggggctgtcgcgggttggggcggttgggctggcagctgaggctcgtggccatggagtggggttctgagtcggctgctgtgaggcggcaccgcgtcggagtagagcgtcgagagggagcggcggccgcgccaccgccggagagggaggcccgagcgcaggagcctctggtggatgggtgcagcggcggcgggaggacgcggaagaggagccccgggggtagcggcggcgcgagcaggggcgcggggaccgggctgtctgaggtgcgcgccgcgctggggctcgcgctctacctgatcgcgctgcggacgctggtgcagctctcgctgcagcagctcgtgctacgcggggccgctggacaccgcggggagttcgacgcgctccaagccagggattatcttgaacacataacctccattggccccaggactacaggaagtccagaaaatgaaattctgaccgtgcactaccttttggaacagattaaactgattgaagtgcaaagcaacagccttcataagatttcagtagatgtacaacggcccacaggctcttttagcattgatttcttgggaggttttacaagctattatgacaacatcaccaatgttgtggtaaagctggaacccagagatggagcccagcatgctgtcttggctaattgtcattttgactcagtagcaaactcaccaggtgccagtgatgatgcagttagctgctcagtgatgctggaagtccttcgcgtcttgtcaacatcttcagaagccttgcatcatgctgtcatatttctctttaatggtgctgaggaaaatgtcttgcaagccagtcatggtttcattactcagcacccctgggctagcttgattcgtgcattcattaacctagaggcagcaggtgtaggagggaaagaacttgtattccaaacaggtcctgaaaatccttggttggttcaagcttatgtttcagcagctaaacacccttttgcttctgtggtggctcaggaggtttttcagagtggaatcattccttcagatactgactttcgtatctacagggattttgggaacattccaggaatagacttagcttttattgagaatggatacatttatcacaccaagtatgacacagcggacagaattctaacagattccattcagagagcaggtgacaacattttagcagttcttaagcatctagctacatctgatatgctggctgctgcttctaagtatcgacatggaaacatggtcttctttgatgtgctgggcctgtttgtcattgcctacccctctcgtattggctcaatcataaactacatggtggtaatgggtgttgttttgtacctgggcaaaaaatttttgcagcccaaacataagactggtaactacaagaaggacttcttgtgtggacttggcatcactttgatcagctggttcactagccttgttaccgttctcattatagcagtgttcatctctcttattggacagtctctctcatggtataaccacttctatgtctccgtttgtctgtatggaactgcaactgtagccaaaataatacttatacatactcttgcgaaaagattttattacatgaatgccagtgcccagtatctgggagaagtattttttgacatttcgctgtttgtccattgctgttttcttgttaccctcacttaccaaggactttgctcggcgtttattagtgctgtctgggtagcattcccattgctcacaaagctctgtgtgcataaggacttcaagcagcatggtgcccaaggaaaatttattgctttttaccttttggggatgtttattccttatctttatgcattgtacctcatctgggcagtatttgagatgtttacccctatcctcgggagaagtggttctgaaatcccacctgatgttgtgctggcatccattttggctggctgtacaatgattctctcgtcctattttattaacttcatctaccttgccaagagcacaaaaaaaaccatgctaactttaactttggtatgtgcaattacattcctccttgtttgcagtggaacattttttccatatagctccaatcctgctaatccgaagccaaagagagtgtttcttcagcatatgactagaacattccatgacttggaaggaaatgcagttaaacgggactctggaatatggatcaatgggtttgattatactggaatttctcacataacccctcacattcctgagatcaatgatagtatccgagctcactgtgaggagaatgcacctctttgtggttttccttggtatcttccagtgcactttctgatcaggaaaaactggtatcttcctgccccagaagtttctccaagaaatcctcctcatttccgactcatatccaaagaacagacaccttgggattctataaaattgacttttgaagcaacaggaccaagccatatgtccttctatgttcgagcccacaaagggtcaacactttctcagtggtctcttggcaatggcaccccagtcacaagtaaaggaggagactactttgtcttttactcccatggactccaggcctctgcatggcagttctggatagaagtgcaggtttcagaagaacatcctgaaggaatggtcaccgtggccattgctgcccactatctgtctggggaagacaagagatcccctcaactggatgctctgaaggaaaagttcccagattggacatttccctctgcctgggtgtgcacctacgatctctttgtattttaatcttgtggatgagctctaagtacatgcccagtggatactccatgtgacatggtttctccctatgttacgtggatgtttgtaacgtaagtcaatgaattttaatgatcatatgttcaaagagctttctgggttaacgcttttcagggccaagcactataagggtttagctgtggcgcagtgatgcatggcctgttgacacttgaaaatgccagtcttttggcacttcagcacatgtgggtactgccactacacacacgtcattttatatgaccttaaggacaaagccaacaatccacttcaatagctgcccctttaggatcaagaaagatgtacactgtcagagcattgttaatgagacaaaagttgtttccaatttaagccccaaaaccatttgttgtattagtggatggtgggtaaaatatcattcactgaggtaatgattccccttgagaatataactctgtgtaggtcactggaaagtgattgccatagggctgggagagaagcattgcactcttgaggctgtagcctgtgtcaagctgtttcttcaggcagcctctcaaatgtgctttgtctctctgtgctgaggcctggaccctgtgctgagctggtgactcactgtcctgacaagtggacacacagatgcactgctgtgctgctttcctgaggtggttttctatgcctgttttcctctgaaacatgtctgttacccctctccatcttaccaagttgaaaaggggaatatttggccacatacccctctggttttcgtaggttcttttggttcagaatattgtttgtgccagtacatgaccttaacttccttcctcagagcactgagctgccatctgggctattctggggtagaaggaaggctgggagtggtgggaattttataaatatttattctcttttctttgtttcataggagtcttgtgttatacaaggttagtccttcatggtataatcttactgatgcactgggcctatctttttgttttccagccagttgaatagattagtttttctcagtaacttactatccagcagactggctttcctgagacttgaggttgtggcttatactggaatgagaccactgtacgtgtaggtggttcagatcctgcgtaatggcagcatgaggacttaaaaggtggttttcattttgaagatggctatgtagcttgtaaggtgtatcacagcagtacctctcatggctttttggttccagcagtgagggcattggtgagatcaatggtaaactgtgcaagctttctttttatcattaggaaatgtgaaacgttggacaaattttgagttttaacaaggacaaaaagttgaaagaaaaggcacagttaacaaaaaagggtggctagatttatcttgggtgatggaggaaatgagagaggaatgctcttgaaaggtggtctgtggatctgtctgaatagaaagagcacagtaagtatgcattgccggagaaaacgtccttgaagctgcttgtctcatgtgtatgatgtgctttttaaatcatgcccctcgttgcctgcctaatctgtgactccctaaaaactaactgggcccatgtagatggggctgcaaccagagctgaataacatgttaggctcacacatgcatcagcactgcacactggaatcattgctcttcctggactttgtagaaatcagtctcaagtgcttcaagagtctggctcctgctacttttatctgtcaggtagcacataaggtttgcagggtttatattttgtatagaatcacagttgtggagaaaaagtaataatttctcaatgaattttaaaaatgggcctattttctatccccgtggttcatctgatataattagtgttccctgtgaattccccccctctatgggaaggatgcctttactctttatcagtaataaattatgactgttttcatattgccttagggttatttccctgtgtaaaccattgtcttttgttttggttttctttagcattatgaagctttggtattgtacaaggtcagtagtaagatgctcactagtctcagggcttgtgtaatattctgggaggtcatttaaatgccagaaatggtcaagcaattatacacagtatttatgactctgttaagcataccgtttgtctgtcacattagtagattctgagattaaaaaaaatttttaaagagtgatcatttaaataatttctaaaagggtcttttcaagctctaacaaagtcactaacaaatgcattattttctacagaattagatgttagtagtacagtactgcatattcagggaaaaagtgtgaggaattgatttcaaaatagttcgttcttgtgtttgacctaagaatgattgtcgcatgaagtgtttgtttttacagtttagcatatataaacaaacatgataggattccttaagatgttaccacccagggggccacaagccagcctgctgtctcaggaagctgtagaaggagtgtttgtcaatttcttgtcactggtttgctgacttactgaggattaattgttgccttacaatgttactgaaataaactgtttaat

SEQ ID NO: 78 - DNA, 5387 bp, Homo sapiens
ggccggggctgtcgcgggttggggcggttgggctggcagctgaggctcgtggccatggagtggggttctgagtcggctgctgtgaggcggcaccgcgtcggagtagagcgtcgagagggagcggcggccgcgccaccgccggagagggaggcccgagcgcaggagcctctggtggatgggtgcagcggcggcgggaggacgcggaagaggagccccgggggtagcggcggcgcgagcaggggcgcggggaccgggctgtctgaggtgcgcgccgcgctggggctcgcgctctacctgatcgcgctgcggacgctggtgcagctctcgctgcagcagctcgtgctacgcggggccgctggacaccgcggggagttcgacgcgctccaagccagggattatcttgaacacataacctccattggccccaggactacaggaagtccagaaaatgaaattctgaccgtgcactaccttttggaacagattaaactgattgaagtgcaaagcaacagccttcataagatttcagtagatgtacaacggcccacaggctcttttagcattgatttcttgggaggttttacaagctattatgacaacatcaccaatgttgtggtaaagctggaacccagagatggagcccagcatgctgtcttggctaattgtcattttgactcagtagcaaactcaccaggtgccagtgatgatgcagttagctgctcagtgatgctggaagtccttcgcgtcttgtcaacatcttcagaagccttgcatcatgctgtcatatttctctttaatggtgctgaggaaaatgtcttgcaagccagtcatggtttcattactcagcacccctgggctagcttgattcgtgcattcattaacctagaggcagcaggtgtaggagggaaagaacttgtattccaaacaggtcctgaaaatccttggttggttcaagcttatgtttcagcagctaaacacccttttgcttctgtggtggctcaggaggtttttcagagtggaatcattccttcagatactgactttcgtatctacagggattttgggaacattccaggaatagacttagcttttattgagaatggatacatttatcacaccaagtatgacacagcggacagaattctaacagattccattcagagagcaggtgacaacattttagcagttcttaagcatctagctacatctgatatgctggctgctgcttctaagtatcgacatggaaacatggtcttctttgatgtgctgggcctgtttgtcattgcctacccctctcgtattggctcaatcataaactacatggtggtaatgggtgttgttttgtacctgggcaaaaaatttttgcagcccaaacataagactggtaactacaagaaggacttcttgtgtggacttggcatcactttgatcagctggttcactagccttgttaccgttctcattatagcagtgttcatctctcttattggacagtctctctcatggtataaccacttctatgtctccgtttgtctgtatggaactgcaactgtagccaaaataatacttatacatactcttgcgaaaagattttattacatgaatgccagtgcccagtatctgggagaagtattttttgacatttcgctgtttgtccattgctgttttcttgttaccctcacttaccaaggactttgctcggcgtttattagtgctgtctgggtagcattcccattgctcacaaagctctgtgtgcataaggacttcaagcagcatggtgcccaaggaaaatttattgctttttaccttttggggatgtttattccttatctttatgcattgtacctcatctgggcagtatttgagatgtttacccctatcctcgggagaagtggttctgaaatcccacctgatgttgtgctggcatccattttggctggctgtacaatgattctctcgtcctattttattaacttcatctaccttgccaagagcacaaaaaaaaccatgctaactttaactttggtatgtgcaattacattcctccttgtttgcagtggaacattttttccatatagctccaatcctgctaatccgaagccaaagagagtgtttcttcagcatatgactagaacattccatgacttggaaggaaatgcagttaaacgggactctggaatatggatcaatgggtttgattatactggaatttctcacataacccctcacattcctgagatcaatgatagtatccgagctcactgtgaggagaatgcacctctttgtggttttccttggtatcttccagtgcactttctgatcaggaaaaactggtatcttcctgccccagaagtttctccaagaaatcctcctcatttccgactcatatccaaagaacagacaccttgggattctataaaattgacttttgaagcaacagcctgcctgcctatccttcagattttggacttgccagcctcaacaatcatgaccaagccatatgtccttctatgttcgagcccacaaagggtcaacactttctcagtggtctcttggcaatggcaccccagtcacaagtaaaggaggagactactttgtcttttactcccatggactccaggcctctgcatggcagttctggatagaagtgcaggtttcagaagaacatcctgaaggaatggtcaccgtggccattgctgcccactatctgtctggggaagacaagagatcccctcaactggatgctctgaaggaaaagttcccagattggacatttccctctgcctgggtgtgcacctacgatctctttgtattttaatcttgtggatgagctctaagtacatgcccagtggatactccatgtgacatggtttctccctatgttacgtggatgtttgtaacgtaagtcaatgaattttaatgatcatatgttcaaagagctttctgggttaacgcttttcagggccaagcactataagggtttagctgtggcgcagtgatgcatggcctgttgacacttgaaaatgccagtcttttggcacttcagcacatgtgggtactgccactacacacacgtcattttatatgaccttaaggacaaagccaacaatccacttcaatagctgcccctttaggatcaagaaagatgtacactgtcagagcattgttaatgagacaaaagttgtttccaatttaagccccaaaaccatttgttgtattagtggatggtgggtaaaatatcattcactgaggtaatgattccccttgagaatataactctgtgtaggtcactggaaagtgattgccatagggctgggagagaagcattgcactcttgaggctgtagcctgtgtcaagctgtttcttcaggcagcctctcaaatgtgctttgtctctctgtgctgaggcctggaccctgtgctgagctggtgactcactgtcctgacaagtggacacacagatgcactgctgtgctgctttcctgaggtggttttctatgcctgttttcctctgaaacatgtctgttacccctctccatcttaccaagttgaaaaggggaatatttggccacatacccctctggttttcgtaggttcttttggttcagaatattgtttgtgccagtacatgaccttaacttccttcctcagagcactgagctgccatctgggctattctggggtagaaggaaggctgggagtggtgggaattttataaatatttattctcttttctttgtttcataggagtcttgtgttatacaaggttagtccttcatggtataatcttactgatgcactgggcctatctttttgttttccagccagttgaatagattagtttttctcagtaacttactatccagcagactggctttcctgagacttgaggttgtggcttatactggaatgagaccactgtacgtgtaggtggttcagatcctgcgtaatggcagcatgaggacttaaaaggtggttttcattttgaagatggctatgtagcttgtaaggtgtatcacagcagtacctctcatggctttttggttccagcagtgagggcattggtgagatcaatggtaaactgtgcaagctttctttttatcattaggaaatgtgaaacgttggacaaattttgagttttaacaaggacaaaaagttgaaagaaaaggcacagttaacaaaaaagggtggctagatttatcttgggtgatggaggaaatgagagaggaatgctcttgaaaggtggtctgtggatctgtctgaatagaaagagcacagtaagtatgcattgccggagaaaacgtccttgaagctgcttgtctcatgtgtatgatgtgctttttaaatcatgcccctcgttgcctgcctaatctgtgactccctaaaaactaactgggcccatgtagatggggctgcaaccagagctgaataacatgttaggctcacacatgcatcagcactgcacactggaatcattgctcttcctggactttgtagaaatcagtctcaagtgcttcaagagtctggctcctgctacttttatctgtcaggtagcacataaggtttgcagggtttatattttgtatagaatcacagttgtggagaaaaagtaataatttctcaatgaattttaaaaatgggcctattttctatccccgtggttcatctgatataattagtgttccctgtgaattccccccctctatgggaaggatgcctttactctttatcagtaataaattatgactgttttcatattgccttagggttatttccctgtgtaaaccattgtcttttgttttggttttctttagcattatgaagctttggtattgtacaaggtcagtagtaagatgctcactagtctcagggcttgtgtaatattctgggaggtcatttaaatgccagaaatggtcaagcaattatacacagtatttatgactctgttaagcataccgtttgtctgtcacattagtagattctgagattaaaaaaaatttttaaagagtgatcatttaaataatttctaaaagggtcttttcaagctctaacaaagtcactaacaaatgcattattttctacagaattagatgttagtagtacagtactgcatattcagggaaaaagtgtgaggaattgatttcaaaatagttcgttcttgtgtttgacctaagaatgattgtcgcatgaagtgtttgtttttacagtttagcatatataaacaaacatgataggattccttaagatgttaccacccagggggccacaagccagcctgctgtctcaggaagctgtagaaggagtgtttgtcaatttcttgtcactggtttgctgacttactgaggattaattgttgccttacaatgttactgaaataaactgtttaat

SEQ ID NO: 79 - protein, 245 aa, Homo sapiens
MTLFPVLLFLVAGLLPSFPANEDKDPAFTALLTTQTQVQREIVNKHNELRRAVSPPARNMLKMEWNKEAAANAQKWANQCNYRHSNPKDRMTSLKCGENLYMSSASSSWSQAIQSWFDEYNDFDFGVGPKTPNAVVGHYTQVVWYSSYLVGCGNAYCPNQKVLKYYYVCQYCPAGNWANRLYVPYEQGAPCASCPDNCDDGLCTNGCKYEDLYSNCKSLKLTLTCKHQLVRDSCKASCNCSNSIY

SEQ ID NO: 80 - protein, 245 aa, Homo sapiens; identical to SEQ ID NO: 79

SEQ ID NO: 81 - protein, 245 aa, Homo sapiens; identical to SEQ ID NO: 79

SEQ ID NO: 82 - DNA, 2073 bp, Homo sapiens
cccagcaatgacattattcccagtgctgttgttcctggttgctgggctgcttccatcttttccagcaaatgaagataaggatcccgcttttactgctttgttaaccacccaaacacaagtgcaaagggagattgtgaataagcacaatgaactgaggagagcagtatctccccctgccagaaacatgctgaagatggaatggaacaaagaggctgcagcaaatgcccaaaagtgggcaaaccagtgcaattacagacacagtaacccaaaggatcgaatgacaagtctaaaatgtggtgagaatctctacatgtcaagtgcctccagctcatggtcacaagcaatccaaagctggtttgatgagtacaatgattttgactttggtgtagggccaaagactcccaacgcagtggttggacattatacacaggttgtttggtactcttcatacctcgttggatgtggaaatgcctactgtcccaatcaaaaagttctaaaatactactatgtttgccaatattgtcctgctggtaattgggctaatagactatatgtcccttatgaacaaggagcaccttgtgccagttgcccagataactgtgacgatggactatgcaccaatggttgcaagtacgaagatctctatagtaactgtaaaagtttgaagctcacattaacctgtaaacatcagttggtcagggacagttgcaaggcctcctgcaattgttcaaacagcatttattaaatacgcattacacaccgagtagggctatgtagagaggagtcagattatctacttagatttggcatctacttagatttaacatatactagctgagaaattgtaggcatgtttgatacacatttgatttcaaatgtttttcttctggatctgctttttattttacaaaaatatttttcatacaaatggttaaaaagaaacaaaatctataacaacaactttggatttttatatataaactttgtgatttaaatttactgaatttaattagggtgaaaattttgaaagttgtattctcatatgactaagttcactaaaaccctggattgaaagtgaaaattatgttcctagaacaaaatgtacaaaaagaacaatataattttcacatgaacccttggctgtagttgcctttcctagctccactctaaggctaagcatcttcaaagacgttttcccatatgctgtcttaattcttttcactcattcacccttcttcccaatcatctggctggcatcctcacaattgagttgaagctgttcctcctaaaacaatcctgacttttattttgccaaaatcaatacaatcctttgaattttttatctgcataaattttacagtagaatatgatcaaaccttcatttttaaacctctcttctctttgacaaaacttccttaaaaaagaatacaagataatataggtaaataccctccactcaaggaggtagaactcagtcctctcccttgtgagtcttcactaaaatcagtgactcacttccaaagagtggagtatggaaagggaaacatagtaactttacaggggagaaaaatgacaaatgacgtcttcaccaagtgatcaaaattaacgtcaccagtgataagtcattcagatttgttctagataatctttctaaaaattcataatcccaatctaattatgagctaaaacatccagcaaactcaagttgaaggacattctacaaaatatccctggggtattttagagtattcctcaaaactgtaaaaatcatggaaaataagggaatcctgagaaacaatcacagaccacatgagactaaggagacatgtgagccaaatgcaatgtgcttcttggatcagatcctggaacagaaaaagatcagtaatgaaaaaactgatgaagtctgaatagaatctggagtatttttaacagtagtgttgatttcttaatcttgataaatatagcagggtaatgtaagatgataacgttagagaaactgaaactgggtgagggctatctaggaattctctgtactatcttaccaaattttcggtaagtctaagaaagcaatgc

SEQ ID NO: 83 - DNA, 2081 bp, Homo sapiens
ctggaaaccactgcaatgacattattcccagtgctgttgttcctggttgctgggctgcttccatcttttccagcaaatgaagataaggatcccgcttttactgctttgttaaccacccaaacacaagtgcaaagggagattgtgaataagcacaatgaactgaggagagcagtatctccccctgccagaaacatgctgaagatggaatggaacaaagaggctgcagcaaatgcccaaaagtgggcaaaccagtgcaattacagacacagtaacccaaaggatcgaatgacaagtctaaaatgtggtgagaatctctacatgtcaagtgcctccagctcatggtcacaagcaatccaaagctggtttgatgagtacaatgattttgactttggtgtagggccaaagactcccaacgcagtggttggacattatacacaggttgtttggtactcttcatacctcgttggatgtggaaatgcctactgtcccaatcaaaaagttctaaaatactactatgtttgccaatattgtcctgctggtaattgggctaatagactatatgtcccttatgaacaaggagcaccttgtgccagttgcccagataactgtgacgatggactatgcaccaatggttgcaagtacgaagatctctatagtaactgtaaaagtttgaagctcacattaacctgtaaacatcagttggtcagggacagttgcaaggcctcctgcaattgttcaaacagcatttattaaatacgcattacacaccgagtagggctatgtagagaggagtcagattatctacttagatttggcatctacttagatttaacatatactagctgagaaattgtaggcatgtttgatacacatttgatttcaaatgtttttcttctggatctgctttttattttacaaaaatatttttcatacaaatggttaaaaagaaacaaaatctataacaacaactttggatttttatatataaactttgtgatttaaatttactgaatttaattagggtgaaaattttgaaagttgtattctcatatgactaagttcactaaaaccctggattgaaagtgaaaattatgttcctagaacaaaatgtacaaaaagaacaatataattttcacatgaacccttggctgtagttgcctttcctagctccactctaaggctaagcatcttcaaagacgttttcccatatgctgtcttaattcttttcactcattcacccttcttcccaatcatctggctggcatcctcacaattgagttgaagctgttcctcctaaaacaatcctgacttttattttgccaaaatcaatacaatcctttgaattttttatctgcataaattttacagtagaatatgatcaaaccttcatttttaaacctctcttctctttgacaaaacttccttaaaaaagaatacaagataatataggtaaataccctccactcaaggaggtagaactcagtcctctcccttgtgagtcttcactaaaatcagtgactcacttccaaagagtggagtatggaaagggaaacatagtaactttacaggggagaaaaatgacaaatgacgtcttcaccaagtgatcaaaattaacgtcaccagtgataagtcattcagatttgttctagataatctttctaaaaattcataatcccaatctaattatgagctaaaacatccagcaaactcaagttgaaggacattctacaaaatatccctggggtattttagagtattcctcaaaactgtaaaaatcatggaaaataagggaatcctgagaaacaatcacagaccacatgagactaaggagacatgtgagccaaatgcaatgtgcttcttggatcagatcctggaacagaaaaagatcagtaatgaaaaaactgatgaagtctgaatagaatctggagtatttttaacagtagtgttgatttcttaatcttgataaatatagcagggtaatgtaagatgataacgttagagaaactgaaactgggtgagggctatctaggaattctctgtactatcttaccaaattttcggtaagtctaagaaagcaatgc

SEQ ID NO: 84 - DNA, 2122 bp, Homo sapiens
gatgaaacaaatacttcatcctgctctggaaaccactgatgacattattcccagtgctgttgttcctggttgctgggctgcttccatcttttccagcaaatgaagataaggatcccgcttttactgctttgttaaccacccaaacacaagtgcaaagggagattgtgaataagcacaatgaactgaggagagcagtatctccccctgccagaaacatgctgaagatggaatggaacaaagaggctgcagcaaatgcccaaaagtgggcaaaccagtgcaattacagacacagtaacccaaaggatcgaatgacaagtctaaaatgtggtgagaatctctacatgtcaagtgcctccagctcatggtcacaagcaatccaaagctggtttgatgagtacaatgattttgactttggtgtagggccaaagactcccaacgcagtggttggacattatacacaggttgtttggtactcttcatacctcgttggatgtggaaatgcctactgtcccaatcaaaaagttctaaaatactactatgtttgccaatattgtcctgctggtaattgggctaatagactatatgtcccttatgaacaaggagcaccttgtgccagttgcccagataactgtgacgatggactatgcaccaatggttgcaagtacgaagatctctatagtaactgtaaaagtttgaagctcacattaacctgtaaacatcagttggtcagggacagttgcaaggcctcctgcaattgttcaaacagcatttattaaatacgcattacacaccgagtagggctatgtagagaggagtcagattatctacttagatttggcatctacttagatttaacatatactagctgagaaattgtaggcatgtttgatacacatttgatttcaaatgtttttcttctggatctgctttttattttacaaaaatatttttcatacaaatggttaaaaagaaacaaaatctataacaacaactttggatttttatatataaactttgtgatttaaatttactgaatttaattagggtgaaaattttgaaagttgtattctcatatgactaagttcactaaaaccctggattgaaagtgaaaattatgttcctagaacaaaatgtacaaaaagaacaatataattttcacatgaacccttggctgtagttgcctttcctagctccactctaaggctaagcatcttcaaagacgttttcccatatgctgtcttaattcttttcactcattcacccttcttcccaatcatctggctggcatcctcacaattgagttgaagctgttcctcctaaaacaatcctgacttttattttgccaaaatcaatacaatcctttgaattttttatctgcataaattttacagtagaatatgatcaaaccttcatttttaaacctctcttctctttgacaaaacttccttaaaaaagaatacaagataatataggtaaataccctccactcaaggaggtagaactcagtcctctcccttgtgagtcttcactaaaatcagtgactcacttccaaagagtggagtatggaaagggaaacatagtaactttacaggggagaaaaatgacaaatgacgtcttcaccaagtgatcaaaattaacgtcaccagtgataagtcattcagatttgttctagataatctttctaaaaattcataatcccaatctaattatgagctaaaacatccagcaaactcaagttgaaggacattctacaaaatatccctggggtattttagagtattcctcaaaactgtaaaaatcatggaaaataagggaatcctgagaaacaatcacagaccacatgagactaaggagacatgtgagccaaatgcaatgtgcttcttggatcagatcctggaacagaaaaagatcagtaatgaaaaaactgatgaagtctgaatagaatctggagtatttttaacagtagtgttgatttcttaatcttgataaatatagcagggtaatgtaagatgataacgttagagaaactgaaactgggtgagggctatctaggaattctctgtactatcttaccaaattttcggtaagtctaagaaagcaatgcaaaataaaaagtgtcttg

SEQ ID NO: 85 - protein, 409 aa, Homo sapiens
MEESWEAAPGGQAGAELPMEPVGSLVPTLEQPQVPAKVRQPEGPESSPSPAGAVEKAAGAGLEPSSKKKPPSPRPGSPRVPPLSLGYGVCPEPPSPGPALVKLPRNGEAPGAEPAPSAWAPMELQVDVRVKPVGAAGGSSTPSPRPSTRFLKVPVPESPAFSRHADPAHQLLLRAPSQGGTWGRRSPLAAARTESGCDAEGRASPAEGSAGSPGSPTCCRCKELGLEKEDAALLPRAGLDGDEKLPRAVTLTGLPMYVKSLYWALAFMAVLLAVSGVVIVVLASRAGARCQQCPPGWVLSEEHCYYFSAEAQAWEASQAFCSAYHATLPLLSHTQDFLGRYPVSRHSWVGAWRGPQGWHWIDEAPLPPQLLPEDGEDNLDINCGALEEGTLVAANCSTPRPWVCAKGTQ

SEQ ID NO: 86 - protein, 314 aa, Homo sapiens
MEESWEAAPGGQAGAELPMEPVGSLVPTLEQPQVPAKVRQPEGPESSPSPAGAVEKAAGAGLEPSSKKKPPSPRPGSPRVPPLSLGYGVCPEPPSPGPALVKLPRNGEAPGAEPAPSAWAPMELQVDVRVKPVGAAGGSSTPSPRPSTRFLKVPVPESPAFSRHADPAHQLLLRAPSQGGTWGRRSPLAAARTESGCDAEGRASPAEGSAGSPGSPTCCRCKELGLEKEDAALLPRAGLDGDEKLPRAVTLTDSLRTARTIWISTVGPWRKARWWLQTAALQDPGSVPRGPSDLGSAWSSACQADAAPPTGEAS

SEQ ID NO: 87 - DNA, 1544 bp, Homo sapiens
gagagcgaagctcctctgcactgggcccaggtgcgctcctcagcgtctccgggtggcggggcgcgcgggatggaggagtcttgggaggctgcgcccggaggccaagccggggcagagctcccaatggagcccgtgggaagcctggtccccacgctggagcagccgcaggtgcccgcgaaggtgcgacaacctgaaggtcccgaaagcagcccaagtccggccggggccgtggagaaggcggcgggcgcaggcctggagccctcgagcaagaaaaagccgccttcgcctcgccccgggtccccgcgcgtgccgccgctcagcctgggctacggggtctgccccgagccgccgtcaccgggccctgccttggtcaagctgccccggaatggcgaggcgcccggggctgagcctgcgcccagcgcctgggcgcccatggagctgcaggtagatgtgcgcgtgaagcccgtgggcgcggccggtggcagcagcacgccatcgcccaggccctccacgcgcttcctcaaggtgccggtgcccgagtcccctgccttctcccgccacgcggacccggcgcaccagctcctgctgcgcgcaccatcccagggcggcacgtggggccgccgctcgccgctggctgcagcccggacggagagcggctgcgacgcagagggccgggccagccccgcggaaggaagcgccggctccccgggctcccccacgtgctgccgctgcaaggagctggggctggagaaggaggatgcggcgctgttgccccgcgcggggttggacggcgacgagaagctgccccgggccgtaacgcttacggggctacccatgtacgtgaagtccctgtactgggccctggcgttcatggctgtgctcctggcagtctctggggttgtcattgtggtcctggcctcaagagcaggagccagatgccagcagtgccccccaggctgggtgttgtccgaggagcactgttactacttctctgcagaagcgcaggcctgggaagccagccaggctttctgctcagcctaccacgctaccctccccctgctaagccacacccaggacttcctgggcagatacccagtctccaggcactcctgggtgggggcctggcgaggcccccagggctggcactggatcgacgaggccccactcccgccccagctactccctgaggacggcgaggacaatctggatatcaactgtggggccctggaggaaggcacgctggtggctgcaaactgcagcactccaagaccctgggtctgtgccaaggggacccagtgatctgggctctgcctggtcctcagcctgccaggcagatgcagcaccccctacaggggaggccagttgagagcttgggcagcctcttcctggacccagttatccaggtcttcatgctctgctcaagggggccacatgagcgagcctaggagctggacttcaacccaggaagatgcatccgagggaaaggagattttctatggcctcaggcctgagtgccaatattagtctccagcttctgtggatga

SEQ ID NO: 88 - DNA, 1192 bp, Homo sapiens
gagagcgaagctcctctgcactgggcccaggtgcgctcctcagcgtctccgggtggcggggcgcgcgggatggaggagtcttgggaggctgcgcccggaggccaagccggggcagagctcccaatggagcccgtgggaagcctggtccccacgctggagcagccgcaggtgcccgcgaaggtgcgacaacctgaaggtcccgaaagcagcccaagtccggccggggccgtggagaaggcggcgggcgcaggcctggagccctcgagcaagaaaaagccgccttcgcctcgccccgggtccccgcgcgtgccgccgctcagcctgggctacggggtctgccccgagccgccgtcaccgggccctgccttggtcaagctgccccggaatggcgaggcgcccggggctgagcctgcgcccagcgcctgggcgcccatggagctgcaggtagatgtgcgcgtgaagcccgtgggcgcggccggtggcagcagcacgccatcgcccaggccctccacgcgcttcctcaaggtgccggtgcccgagtcccctgccttctcccgccacgcggacccggcgcaccagctcctgctgcgcgcaccatcccagggcggcacgtggggccgccgctcgccgctggctgcagcccggacggagagcggctgcgacgcagagggccgggccagccccgcggaaggaagcgccggctccccgggctcccccacgtgctgccgctgcaaggagctggggctggagaaggaggatgcggcgctgttgccccgcgcggggttggacggcgacgagaagctgccccgggccgtaacgcttacggactccctgaggacggcgaggacaatctggatatcaactgtggggccctggaggaaggcacgctggtggctgcaaactgcagcactccaagaccctgggtctgtgccaaggggacccagtgatctgggctctgcctggtcctcagcctgccaggcagatgcagcaccccctacaggggaggccagttgagagcttgggcagcctcttcctggacccagttatccaggtcttcatgctctgctcaagggggccacatgagcgagcctaggagctggacttcaacccaggaagatgcatccgagggaaaggagattttctatggcctcaggcctgagtgccaatattagtctccagcttctgtggatga

SEQ ID NO: 89 - DNA, 21 bp, Homo sapiens
cgaggacaatctggatatcaa

SEQ ID NO: 90 - DNA, 21 bp, Homo sapiens
ctggagccctcgagcaagaaa

SEQ ID NO: 91 - DNA, 21 bp, Homo sapiens
cccgtggttcatctgatataa

SEQ ID NO: 92 - DNA, 21 bp, Homo sapiens
aaggactttgctcggcgttta

SEQ ID NO: 93 - DNA, 21 bp, Homo sapiens
tacgtggatgtttgtaacgta

SEQ ID NO: 94 - DNA, 21 bp, Homo sapiens
ctcgtattggctcaatcataa

(19) United States
(12) Patent Application Publication
(10) Pub. No.: US 2013/0017546 A1
(43) Pub. Date: Jan. 17, 2013

(54) Breast Tumor Markers and Methods of Use Thereof

(71) Applicants: Renata Grifantini, Siena (IT); Piero Pileri, Siena (IT); Susanna Campagnoli, Siena (IT); Alberto Grandi, Siena (IT); Matteo Parri, Siena (IT); Andrea Pierleoni, Siena (IT); Renzo Nogarotto, Siena (IT)

(72) Inventors: Renata Grifantini, Siena (IT); Piero Pileri, Siena (IT); Susanna Campagnoli, Siena (IT); Alberto Grandi, Siena (IT); Matteo Parri, Siena (IT); Andrea Pierleoni, Siena (IT); Renzo Nogarotto, Siena (IT)

(73) Assignee: Externautics S.P.A., Siena (IT)

(21) Appl. No.: 13/503,430

(22) PCT Filed: Oct. 26, 2010
(86) PCT No.: PCT/EP2010/066146

§ 371 Date: Sep. 7, 2012

(30) Foreign Application Priority Data
Oct. 26, 2009 (EP) 09174060.5

(51) Int. Cl. G01N 33/574; C12Q 1/68; C07K 16/40; C07K 14/47; C07K 16/18; C12N 9/76; C07H 21/02; C12N 9/64

(52) U.S. Cl. 435/6.12; 536/24.5; 435/226; 435/7.4; 435/7.23; 435/7.92; 435/7.9; 435/7.1; 435/6.13; 530/350; 530/387.9; 435/213

(57) ABSTRACT

Newly identified proteins as markers for the detection of breast tumors, or as therapeutic targets for treatment thereof; affinity ligands capable of selectively interacting with the newly identified markers, as well as methods for tumor diagnosis and therapy using such ligands.

The present invention relates to newly identified proteins as markers for the detection of breast tumors, or as therapeutic targets for treatment thereof. Also provided are affinity ligands capable of selectively interacting with the newly identified markers, as well as methods for tumor diagnosis and therapy using such ligands.

### BACKGROUND OF THE INVENTION

Tumor Markers (or Biomarkers)

Tumor markers are substances that can be produced by tumor cells or by other cells of the body in response to cancer. In particular, a protein biomarker is either a single protein or a panel of different proteins, that could be used to unambiguously distinguish a disease state. Ideally, a biomarker would have both a high specificity and sensitivity, being represented in a significant percentage of the cases of given disease and not in healthy state.

Biomarkers can be identified in different biological samples, like tissue biopsies or preferably biological fluids (saliva, urine, blood-derivatives and other body fluids), whose collection does not necessitate invasive treatments. Tumor marker levels may be categorized in three major classes on the basis of their clinical use. Diagnostic markers can be used in the detection and diagnosis of cancer. Prognostics markers are indicative of specific outcomes of the disease and can be used to define predictive models that allow the clinicians to predict the likely prognosis of the disease at time of diagnosis. Moreover, prognosis markers are helpful to monitor the patient response to a drug therapy and facilitate a more personalized patient management. A decrease or return to a normal level may indicate that the cancer is responding to therapy, whereas an increase may indicate that the cancer is not responding. After treatment has ended, tumor marker levels may be used to check for recurrence of the tumor. Finally, therapeutic markers can be used to develop tumor-specific drugs or affinity ligand (i.e. antibodies) for a prophylactic intervention.

Currently, although an abnormal tumor marker level may suggest cancer, this alone is usually not enough to accurately diagnose cancer and their measurement in body fluids is frequently combined with other tests, such as a biopsy and radioscopic examination. Frequently, tumor marker levels are not altered in all of people with a certain cancer disease, especially if the cancer is at early stage. Some tumor marker levels can also be altered in patients with noncancerous conditions. Most biomarkers commonly used in clinical practice do not reach a sufficiently high level of specificity and sensitivity to unambiguously distinguish a tumor from a normal state.

To date the number of markers that are expressed abnormally is limited to certain types/subtypes of cancer, some of which are also found in other diseases. (http://www.cancer.gov/cancertopics/factsheet).

For instance, the human epidermal growth factor receptor (HER2) is a marker protein overproduced in about 20% of breast cancers, whose expression is typically associated with a more aggressive and recurrent tumors of this class.

Routine Screening Test for Tumor Diagnosis

Screening tests are a way of detecting cancer early, before there are any symptoms. For a screening test to be helpful, it should have high sensitivity and specificity. Sensitivity refers to the test's ability to identify people who have the disease. Specificity refers to the test's ability to identify people who do not have the disease. Different molecular biology approaches such as analysis of DNA sequencing, small nucleotide polymorphyms, in situ hybridization and whole transcriptional profile analysis have done remarkable progresses to discriminate a tumor state from a normal state and are accelerating the knowledge process in the tumor field. However so far different reasons are delaying their use in the common clinical practice, including the higher analysis complexity and their expensiveness. Other diagnosis tools whose application is increasing in clinics include in situ hybridization and gene sequencing.

Currently, Immuno-HistoChemistry (IHC), a technique that allows the detection of proteins expressed in tissues and cells using specific antibodies, is the most commonly used method for the clinical diagnosis of tumor samples. This technique enables the analysis of cell morphology and the classification of tissue samples on the basis of their immunoreactivity. However, at present, IHC can be used in clinical practice to detect cancerous cells of tumor types for which protein markers and specific antibodies are available. In this context, the identification of a large panel of markers for the most frequent cancer classes would have a great impact in the clinical diagnosis of the disease.

Anti-Cancer Therapies

In the last decades, an overwhelming number of studies remarkably contributed to the comprehension of the molecular mechanisms leading to cancer. However, this scientific progress in the molecular oncology field has not been paralleled by a comparable progress in cancer diagnosis and therapy. Surgery and/or radiotherapy are the still the main modality of local treatment of cancer in the majority of patients. However, these treatments are effective only at initial phases of the disease and in particular for solid tumors of epithelial origin, as is the case of colon, lung, breast, prostate and others, while they are not effective for distant recurrence of the disease. In some tumor classes, chemotherapy treatments have been developed, which generally relies on drugs, hormones and antibodies, targeting specific biological processes used by cancers to grow and spread. However, so far many cancer therapies had limited efficacy due to severity of side effects and overall toxicity. Indeed, a major effort in cancer therapy is the development of treatments able to target specifically tumor cells causing limited damages to surrounding normal cells thereby decreasing adverse side effects. Recent developments in cancer therapy in this direction are encouraging, indicating that in some cases a cancer specific therapy is feasible. In particular, the development and commercialization of humanized monoclonal antibodies that recognize specifically tumor-associated markers and promote the elimination of cancer is one of the most promising solutions that appears to be an extremely favorable market opportunity for pharmaceutical companies. However, at present the number of therapeutic antibodies available on the market or under clinical studies is very limited and restricted to specific cancer classes. So far licensed monoclonal antibodies currently used in clinics for the therapy of specific tumor classes, show only a partial efficacy and are frequently associated with chemotherapies to increase their therapeutic effect. Administration of Trastuzumab (Herceptin), a commercial monoclonal antibody targeting HER2, a protein overproduced in about 20% of breast cancers, in conjunction with Taxol adjuvant chemotherapy induces tumor remission in about 42% of the cases. Bevacizumab (Avastin) and Cetuximab (Erbitux) are two monoclonal antibodies recently licensed for use in humans, targeting the endothelial and epithelial growth factors respectively that, combined with adjuvant chemotherapy, proved to be effective against different tumor diseases. Bevacizumab proved to be effective in prolonging the life of patients with metastatic colorectal, breast and lung cancers. Cetuximab demonstrated efficacy in patients with tumor types refractory to standard chemotherapeutic treatments (Adams G. P. and Weiner L. M. (2005) Monoclonal antibody therapy cancer. Nat. Biotechnol. 23:1147-57).

In summary, available screening tests for tumor diagnosis are uncomfortable or invasive and this sometimes limits their applications. Moreover tumor markers available today have a limited utility in clinics due to either their incapability to detect all tumor subtypes of the defined cancers types and/or to distinguish unambiguously tumor vs. normal tissues. Similarly, licensed monoclonal antibodies combined with standard chemotherapies are not effective against the majority of cases. Therefore, there is a great demand for new tools to advance the diagnosis and treatment of cancer.

Experimental Approaches Commonly Used to Identify Tumor Markers

Most popular approaches used to discover new tumor markers are based on genome-wide transcription profile or total protein content analyses of tumor. These studies usually lead to the identification of groups of mRNAs and proteins which are differentially expressed in tumors. Validation experiments then follow to eventually single out, among the hundreds of RNAs/proteins identified, the very few that have the potential to become useful markers. Although often successful, these approaches have several limitations and often, do not provide firm indications on the association of protein markers with tumor. A first limitation is that, since frequently mRNA levels not always correlate with corresponding protein abundance (approx. 50% correlation), studies based on transcription profile do not provide solid information regarding the expression of protein markers in tumor (1, 2, 3, 4).

A second limitation is that neither transcription profiles nor analysis of total protein content discriminate post-translation modifications, which often occur during oncogenesis. These modifications, including phosphorylations, acetylations, and glycosylations, or protein cleavages influence significantly protein stability, localization, interactions, and functions (5).

As a consequence, large scale studies generally result in long lists of differentially expressed genes that would require complex experimental paths in order to validate the potential markers. However, large scale genomic/proteomic studies reporting novel tumor markers frequently lack of confirmation data on the reported potential novel markers and thus do not provide solid demonstration on the association of the described protein markers with tumor.

The approach that we used to identify the protein markers included in the present invention is based on an innovative immuno-proteomic technology. In essence, a library of recombinant human proteins has been produced from E. coli and is being used to generate polyclonal antibodies against each of the recombinant proteins.

The screening of the antibodies library on Tissue microarrays (TMAs) carrying clinical samples from different patients affected by the tumor under investigation leads to the identification of specific tumor marker proteins. Therefore, by screening TMAs with the antibody library, the tumor markers are visualized by immuno-histochemistry, the classical technology applied in all clinical pathology laboratories. Since TMAs also include healthy tissues, the specificity of the antibodies for the tumors can be immediately appreciated and information on the relative level of expression and cellular localization of the markers can be obtained. In our approach the markers are subjected to a validation process consisting in a molecular and cellular characterization.

Altogether, the detection the marker proteins disclosed in the present invention selectively in tumor samples and the subsequent validation experiments leads to an unambiguous confirmation of the marker identity and confirm its association with defined tumor classes. Moreover this process provides an indication of the possible use of the proteins as tools for diagnostic or therapeutic intervention. For instance, markers showing a surface cellular localization could be both diagnostic and therapeutic markers against which both chemical and antibody therapies can be developed. Differently, markers showing a cytoplasmic expression could be more likely considered for the development of tumor diagnostic tests and chemotherapy/small molecules treatments.

### SUMMARY OF THE INVENTION

The present invention provides new means for the detection and treatment of breast tumors, based on the identification of protein markers specific for these tumor types, namely:

i) Endoplasmic reticulum metallopeptidase 1 (ERMP1)

ii) Chromosome 6 open reading frame 98 (C6orf98);

iii) Chromosome 9 open reading frame 46 (c9orf46);

iv) Putative uncharacterized protein (FLJ37107);

v) Yip1 domain family, member 2 (YIPF2);

vi) Uncharacterized protein UNQ6126/PRO20091 (UNQ6126);

vii) Trypsin-X3 Precursor (TRYX3);

viii) DPY-19-like 3 (DPY19L3);

ix) solute carrier family 39 (zinc transporter), member 10 (SLC39A10);

x) Chromosome 14 open reading frame 135 (c14orf135);

xi) DENN/MADD domain containing 1B (DENND1B);

xii) EMI domain-containing protein 1 Precursor (EMID1);

xiii) Cysteine-rich secretory protein 3 Precursor (CRISP3)

xiv) Killer cell lectin-like receptor subfamily G member 2 (C-type lectin domain family 15 member B) (KLRG2).

In preferred embodiments, the invention provides the use of, alone or in combination, C6orf98, C9orf46, FLJ37107, YIPF2, UNQ6126, TRYX3, DPY19L3, SLC39A10, C14orf135; DENND1B, EMID1, ERMP1, CRISP3 and KLRG2 as markers or targets for breast tumor.

The invention also provides a method for the diagnosis of these cancer types, comprising a step of detecting the above-identified markers in a biological sample, e.g. in a tissue sample of a subject suspected of having or at risk of developing malignancies or susceptible to cancer recurrences.

In addition, the tumor markers identify novel targets for affinity ligands, which can be used for therapeutic applications. Also provided are affinity ligands, particularly antibodies, capable of selectively interacting with the newly identified protein markers.

### DETAILED DISCLOSURE OF THE INVENTION

The present invention is based on the surprising finding of antibodies that are able to specifically stain breast tumor tissues from patients, while negative or very poor staining is observed in normal breast tissues from the same patients. These antibodies have been found to specifically bind to proteins for which no previous association with tumor has been reported. Hence, in a first aspect, the invention provides a breast tumor marker, which is selected from the group consisting of:

i) C6orf98 in one of its variant isoforms SEQ ID NO:1, or a different isoform having sequence identity of at least 80%, preferably at least 90%, more preferably at least 95% to SEQ ID NO:1; or a nucleic acid molecule containing a sequence coding for a C6orf98 protein, said encoding sequence being preferably SEQ ID NO: 2;

ii) C9orf46, in one of its variant isoforms SEQ ID NO:3, or a different isoform having sequence identity of at least 80%, preferably at least 90%, more preferably at least 95% to SEQ ID NO:3, or a nucleic acid molecule containing a sequence coding for a C9orf46 protein, said encoding sequence being preferably SEQ ID NO:4;

iii) FLJ37107, in one of its variant isoforms SEQ ID NO:5, or a different isoform having sequence identity of at least 80%, preferably at least 90%, more preferably at least 95% to SEQ ID NO:5; or a nucleic acid molecule containing a sequence coding for a FLJ37107 protein, said encoding sequence being preferably SEQ ID NO: 6;

iv) YIPF2, SEQ ID NO:7, SEQ ID NO:8, or a different isoform having sequence identity of at least 80%, preferably at least 90%, more preferably at least 95% to SEQ ID NO:7 or SEQ ID NO:8, or a nucleic acid molecule containing a sequence coding for a YIPF2 protein, said encoding sequence being preferably selected from SEQ ID NO:9 and SEQ ID NO:10;

v) UNQ6126, in one of its variant isoforms SEQ ID NO:11, or a different isoform having sequence identity of at least 80%, preferably at least 90%, more preferably at least 95% to SEQ ID NO:11, or a nucleic acid molecule containing a sequence coding for a UNQ6126 protein, said encoding sequence being preferably SEQ ID NO: 12;

vi) TRYX3, in one of its variant isoforms SEQ ID NO:13, or a different isoform having sequence identity of at least 80%, preferably at least 90%, more preferably at least 95% to SEQ ID NO:13, or a nucleic acid molecule containing a sequence coding for a TRYX3 protein, said encoding sequence being preferably SEQ ID NO:14;

vii) DPY19L3, in one of its variant isoforms SEQ ID NO:15, SEQ ID NO:16, SEQ ID NO:17, SEQ ID NO:18, or a different isoform having sequence identity of at least 80%, preferably at least 90%, more preferably at least 95% to any of SEQ ID NO:15, SEQ ID NO:16, SEQ ID NO:17 or SEQ ID NO:18, or a nucleic acid molecule containing a sequence coding for a DPY19L3 protein, said encoding sequence being preferably selected from SEQ ID NO:19, SEQ ID NO:20, SEQ ID NO:21, SEQ ID NO:22;

viii) SLC39A10, SEQ ID NO:23, SEQ ID NO:24 or a different isoform having sequence identity of at least 80%, preferably at least 90%, more preferably at least 95% to SEQ ID NO:23 or SEQ ID NO:24, or a nucleic acid molecule containing a sequence coding for a SLC39A10 protein, said encoding sequence being preferably selected from SEQ ID NO:25 and SEQ ID NO:26;

ix) C14orf135, SEQ ID NO:27, SEQ ID NO:28, SEQ ID NO:29, SEQ ID NO:30, SEQ ID NO:31, or a different isoform having sequence identity of at least 80%, preferably at least 90%, more preferably at least 95% to any of SEQ ID NO:27, SEQ ID NO:28, SEQ ID NO:29, SEQ ID NO:30 or SEQ ID NO:31, or a nucleic acid molecule containing a sequence coding for a C14orf135 protein, said encoding sequence being preferably selected from SEQ ID NO:32, SEQ ID NO:33, SEQ ID NO:34, SEQ ID NO:35 and SEQ ID NO:36;

x) DENND1B; in one of its variant isoforms SEQ ID NO:37, SEQ ID NO:38, SEQ ID NO:39, SEQ ID NO:40, or a different isoform having sequence identity of at least 80%, preferably at least 90%, more preferably at least 95% to any of SEQ ID NO:37, SEQ ID NO:38, SEQ ID NO:39 or SEQ ID NO:40, or a nucleic acid molecule containing a sequence coding for a DENND1B protein, said encoding sequence being preferably selected from SEQ ID NO:41, SEQ ID NO:42, SEQ ID NO:43 and SEQ ID NO:44;

xi) EMID1, in one of its variant isoforms SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, or a different isoform having sequence identity of at least 80%, preferably at least 90%, more preferably at least 95% to any of SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57 or SEQ ID NO:58, or a nucleic acid molecule containing a sequence coding for a DENND1B protein, said encoding sequence being preferably selected from SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71 and SEQ ID NO:72;

xii) ERMP1, SEQ ID NO:73, SEQ ID NO:74, SEQ ID NO:75, or a different isoform having sequence identity of at least 80%, preferably at least 90%, more preferably at least 95% to SEQ ID NO:73, SEQ ID NO:74, SEQ ID NO:75, or a nucleic acid molecule containing a sequence coding for a ERMP1, protein, said encoding sequence being preferably selected from SEQ ID NO:76 SEQ ID NO: 77 and SEQ ID NO:78;

xiii) CRISP-3, SEQ ID NO: 79, SEQ ID NO:80, SEQ ID NO:81, or a different isoform having sequence identity of at least 80%, preferably at least 90%, more preferably at least 95% to SEQ ID NO: 79, SEQ ID NO:80, SEQ ID NO:81, or a nucleic acid molecule containing a sequence coding for a CRISP-3, protein, said encoding sequence being preferably selected from SEQ ID NO:82, SEQ ID NO:83 and SEQ ID NO:84.

xiv) KLRG2, SEQ ID: NO 85, SEQ ID NO:86 or a different isoform having sequence identity of at least 80%, preferably at least 90%, more preferably at least 95% to SEQ ID: NO 85 or SEQ ID: NO 86, or a nucleic acid molecule containing a sequence coding for a KLRG2 protein, said encoding sequence being preferably selected from SEQ ID NO: 87 and SEQ ID NO: 88;

As used herein, “Percent (%) amino acid sequence identity” with respect to the marker protein sequences identified herein indicates the percentage of amino acid residues in a full-length protein variant or isoform according to the invention, or in a portion thereof, that are identical with the amino acid residues in the specific marker sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Identity between nucleotide sequences is preferably determined by the Smith-Waterman homology search algorithm as implemented in the MPSRCH program (Oxford Molecular), using an affine gap search with parameters gap open penalty=12 and gap extension penalty=1.

Solute carrier family 39 member 10 (SLC39A10, synonyms: Zinc transporter ZIP10 Precursor, Zrt- and Irt-like protein 10, ZIP-10, Solute carrier family 39 member 10; gene ID: ENSG00000196950; transcript IDs: ENST00000359634, ENST00000409086; protein ID: ENSP00000352655, ENSP00000386766) belongs to a subfamily of proteins that show structural characteristics of zinc transporters. It is an integral membrane protein likely involved in zinc transport. While other members of the zinc transport family have been at least partially studied in tumors, little is known about the association of SLC39A10 with these diseases. SLC39A10 mRNA has been shown to increase moderately in breast cancer tissues as compared to normal samples (approximately 1.5 fold). Loss of SLC39A10 transcription in breast cell lines has been shown to reduce cell migratory activity (6). However, published studies on the expression of SLC39A10 in breast tumor cells are limited to the analysis of SLC39A10 transcript whilst, to the best of our knowledge, no data have been reported documenting the presence of SLC39A10 protein in these tumor cells.

SLC39A10 is mentioned in a patent application reporting long lists of differentially transcribed genes in tumor cells based on the use of genome-scale transcription profile analysis (e.g. in Publication Number: US20070237770A1). However, since mRNA levels not always correlate with protein levels, studies solely based on transcription profile do not provide solid information regarding the expression of protein markers. Moreover, the lack of correlation between mRNA and protein expression has been specifically demonstrated for LIV-1, another member of the zinc transporter family, suggesting that a similar phenomenon could be extended to other proteins of this class (7).

In the present invention we disclose SLC39A10 as a protein without previous known association with breast tumor classes and preferably used as a marker for breast tumors and in general for cancers of these types. As described below, an antibody generated towards the SLC39A10 protein shows a selective immunoreactivity in histological preparation of breast cancer tissues which indicates the presence of SLC39A10 in these cancer samples and makes SLC39A10 protein and its antibody highly interesting tools for specifically distinguishing these cancer types from a normal state. Moreover, we show that the protein is localized on the surface of tumor cell lines, indicating that this protein is an ideal candidate target for anti-tumor therapies.

Chromosome 6 open reading frame 98 (C6orf98; synonym: dJ45H2.2; Gene ID: EG:387079, da ENSG00000222029 has 1 transcript: ENST00000409023, associated peptide: ENSP00000386324 and 1 exon: ENSE00001576965) is an uncharacterized protein. Analysis of human genome databases (E.g. Ensembl) erroneously assigns C6orf98 as SYNE1. Although SYNE nucleic acid sequences overlap with C60RF98 transcript, the encoded proteins show no match. In fact C6orf98 locus maps on an SYNE1 untranslated region (intron) and its product derives from a different reading frame than those annotated for SYNE1 isoforms in public databases. C6orf98 is a protein without previous known association with tumor and is preferably used as a marker for breast tumor and in general for these cancer types. As described below, an antibody generated towards C6orf98 protein shows a selective immune-reactivity in histological preparation of breast cancer tissues, which indicates the presence of this protein in these cancer samples.

Chromosome 9 open reading frame 46 (C9orf46; synonyms: Transmembrane protein C9orf46; Gene ID: ENSG00000107020; Transcript ID:ENST00000223864; Protein ID: ENSP00000223864) is a poorly characterized protein. So far expression of C9orf46 has only been shown at transcriptional level in metastasis in oral squamous cell carcinoma (8) while no data are available on the expression of its encoded product in tumor. Based on available scientific publications, C9orf46 is a protein without previous known association with breast tumor and is preferably used as a marker for breast tumors. As described below, an antibody generated towards C9orf46 protein shows a selective immunoreactivity in histological preparation of breast cancer tissues, which indicates the presence of this protein in these cancer samples.

Putative uncharacterized protein FLJ37107—(FLJ37107; synonyms: LOC284581; Gene ID: ENSG00000177990, Transcript ID: gi|58218993|ref‥NM_{—}001010882.1, Protein ID: gi|58218994|ref|NP_{—}001010882.1| hypothetical protein LOC284581 [Homo sapiens], gi|74729692|sp|Q8N9I1.1|YA028 HUMAN) is an uncharacterized protein without previous known association with tumor and is preferably used as a marker for breast tumor and in general for these cancer types. As described below, an antibody generated towards FLJ37107 protein shows a selective immunoreactivity in histological preparation of breast cancer tissues, which indicates the presence of this protein in these cancer samples.

Yip1 domain family, member 2 (YIPF2; synonyms: FinGER2; Gene ID: ENSG00000130733; Transcript IDs: ENST00000393508, ENST00000253031; Protein IDs: ENSP00000377144, ENSP00000253031) is an uncharacterized without previous known association with tumor and is preferably used as a marker for breast tumor in general for these cancer types. As described below, an antibody generated towards YIPF2 protein shows a selective immunoreactivity in histological preparation of breast cancer tissues, which indicates the presence of this protein in these cancer samples.

Uncharacterized protein UNQ6126/PRO20091 (UNQ6126, LPEQ6126, synonyms: LOC100128818; Gene ID: gi|169216088; Transcript ID: GB:AY358194, Protein ID: SP:Q6UXV3); is an uncharacterized protein without previous known association with tumor and is preferably used as a marker for breast tumor, and in general for cancers of this type. As described below, an antibody generated towards UNQ6126 protein shows a selective immunoreactivity in histological preparation of breast cancer tissues.

A TRYX3 sequence has been listed in several generic patents, in which no solid data are reported showing the association of TRYX3 protein with tumor (e.g. U.S. Pat. No. 7,105,335, U.S. Pat. No. 7,285,626). Based on the above, TRYX3 is a protein without previous known association with tumor and preferably used as a marker for breast tumor and in general for cancers of this type. As described below, an antibody generated towards TRYX3 protein shows a selective immunoreactivity in histological preparation of breast cancer tissues, which indicates the presence of this protein in these cancer samples.

Protein dpy-19 homolog 3 (DPY19L3; synonym: Dpy-19-like protein 3; Gene ID: ENSG00000178904; Transcript IDs: ENST00000319326, ENST00000392250, ENST00000342179, ENST00000392248; Protein IDs: ENSP00000315672, ENSP00000376081. ENSP00000344937, ENSP00000376079) is a poorly characterized characterized protein. DPY19L3 transcript has been reported as differentially expressed in a large-scale study on multiple myeloma (Publication Number: US20080280779A1). However no data are available at level of protein expression. In the present invention we disclose DPY19L3 protein as associated with tumor and preferably used as a marker for breast tumor, and in general for these cancer types. As described below, an antibody generated towards DPY19L3 protein shows a selective immunoreactivity in histological preparation of breast cancer tissues, which indicates the presence of this protein in these cancer samples. Finally the protein is detected on the surface of tumor cell lines by the specific antibody, suggesting that it can be exploited as target for affinity ligands with therapeutic activity.

Chromosome 14 open reading frame 135 (C14orf135, Pecanex-like protein C14orf135, synonyms: Hepatitis C virus F protein-binding protein 2, HCV F protein-binding protein 2; Gene ID: ENSG00000126773; Transcript IDs: ENST00000317623, ENST00000404681; Protein IDs: ENSP00000317396, ENSP00000385713) is a uncharacterized protein. This protein is mentioned in a patent application on ovarian tumor (Publication number: US2006432604A). In the present invention we report C14orf135 as a protein without previous known association with breast tumor class and preferably used as a marker for breast tumor, and in general for cancers of this type. As described below, an antibody generated towards C14orf135 protein shows a selective immunoreactivity in histological preparation of breast cancer tissues, which indicates the presence of this protein in this cancer samples; moreover this antibody also stains plasma membranes of tumor cells, indicating that c14orf135 protein is localized on the cell surface.

DENN/MADD domain containing 1B (DENND1B; synonyms: DENN domain-containing protein 1B, Protein FAM31B, C1orf218; Gene ID: ENSG00000162701. Transcript IDs: ENST00000294738, ENST00000367396, ENST00000400967, ENST00000235453; Protein IDs: ENSP00000294738, ENSP00000356366, ENSP00000383751, ENSP00000235453) is a poorly characterized protein without previous known association with breast tumors and is preferably used as a marker for breast tumor and in general for these cancer types. As described below, an antibody generated towards DENND1B protein shows a selective immunoreactivity in histological preparation of breast cancer tissues, which indicates the presence of this protein in these cancer samples.

EMI domain-containing protein 1 Precursor (EMID1; synonyms: Emilin and multimerin domain-containing protein 1, Protein Emul; Gene ID: >OTTHUMG00000030824

| Transcript IDs: | Protein IDs: |
|---|---|
| OTTHUMT00000075712 | OTTHUMP00000028901, |
| ENST00000429226 | ENSP00000403816, |
| ENST00000430127 | ENSP00000399760, |
| ENST00000435427 | ENSP00000402621, |
| ENST00000404820 | ENSP00000384452, |
| ENST00000334018 | ENSP00000335481, |
| ENST00000429415 | ENSP00000409801, |
| ENST00000448676 | ENSP00000413034, |
| ENST00000404755 | ENSP00000385414, |
| ENST00000435194 | ENSP00000417004, |
| ENST00000426629 | ENSP00000403484, |
| ENST00000457925 | ENSP00000405422, |
| ENST00000433143 | ENSP00000408339, |
| ENST00000455501 | ENSP00000413947), |

is a poorly characterized protein. EMID gene is mentioned in a patent application on follicular thyroid carcinoma (Publication number US2006035244 (A1). However, no data are available on the presence of this protein in breast tumor. Therefore, we disclose EMID1 as a protein without previous known association with breast tumors and preferably used as a marker for breast tumor and in general for these cancer types. As described below, an antibody generated towards EMID1 protein shows a selective immunoreactivity in histological preparation of breast cancer tissues, which indicates the presence of this protein in these cancer samples. In particular this antibody stains tumor secretion products indicating that EMID1 protein is specifically released by tumor cells.

Endoplasmic reticulum metallopeptidase 1 (ERMP1, synonyms: FLJ23309, FXNA, KIAA1815; GENE ID: ENSG00000099219; Transcript IDs: ENST00000214893, ENST00000339450, ENST00000381506; Protein IDs: ENSP00000214893, ENSP00000340427, ENSP00000370917) is a transmembrane metallopeptidase, so far described as localized to the endoplasmic reticulum. ERMP1 transcript has been found differentially expressed in the rat ovary at the time of folliculogenesis. A lower level of ERMP1 transcript in the rat ovary resulted in substantial loss of primordial, primary and secondary follicles, and structural disorganization of the ovary, suggesting that is required for normal ovarian histogenesis (9). ERMP1 has been also included in a patent application (Publication number US 2003064439) on novel nucleic acid sequences encoding melanoma associated antigen molecules, however no solid data documented the expression of ERMP1 protein in tumor. Based on this, ERMP1 protein has never been previously associated with tumor. In the present invention, differently with published scientific data, we disclose ERMP1 as a protein associated with tumor, preferably used as a marker for breast tumor, and in general for cancers of this type. As described below, an antibody generated towards ERMP1 protein shows a selective immunoreactivity in histological preparation of breast cancer tissues, which indicates the presence of this protein in this cancer type. In particular our immunoistochemistry analysis indicates that the protein shows plasma membrane localization in tumor cells.

Moreover, localization analysis of ovary tumor cell lines showed that this proteins is exposed on the cell surface and accessible to the binding of specific antibodies. Finally, silencing of ERMP1 gene significantly reduced the invasiveness and proliferation properties of tumor cells lines. Based on the above evidences, ERMP1 is a likely target for the development of anti-cancer therapies being exposed to the action of affinity ligand and being involved in cellular processes relevant for tumor development.

Cysteine-rich secretory protein 3 Precursor (CRISP-3, synonyms: SGP28 protein; GENE ID: ENSG00000096006; Transcript IDs: ENST00000393666, ENST00000371159, ENST00000263045; Protein IDs: ENSP00000377274, ENSP00000360201, ENSP00000263045) is also known as specific granule protein of 28 KDa. In humans, high level of CRISP3 transcript protein has been detected in salivary glands, pancreas and prostate, while low expression was found in other tissues such as epidydimis, ovary, thymus and colon (10). Upregulation of CRISP3 has been shown in malignant prostatic epithelium at RNA and protein level. Strong immunostaining for CRISP3 has been associated with high-grade prostatic-intraepithelial-neoplasia and preserved in prostatic cancer (11). CRISP3 has been proposed as predictor of recurrence after radical prostatectomy for localized prostate cancer (12). While CRISP3 protein has been detected and largely characterized in prostate tumor, no previous data exist on its association with breast tumor. In the present invention we disclose CRISP3 as a marker for breast tumor, and in general for cancers of this type. As described below, an antibody generated towards CRISP3 protein shows a selective immunoreactivity in histological preparation of breast cancer tissues, which indicates the presence of this protein in this cancer type.

Killer cell lectin-like receptor subfamily G member 2 (C-type lectin domain family 15 member B) (KLRG2, synonyms: CLEC15B, FLJ44186; GENE ID: ENSG00000188883; Transcript IDs: ENST00000340940, ENST00000393039; Protein IDs: ENSP00000339356, ENSP00000376759) is a poorly uncharacterized protein. A KLRG2 sequence is included in a patent application on the use of an agent with tumor-inhibiting action of a panel of targets associated with different tumors, whose expression is mainly shown at RNA level (Publication number WO2005030250). However no data are provided documenting the presence of KLRG2 protein in the tumors. Moreover, no experimental evidence is given on the specificity of the proposed anti-tumor agent for KLRG2. Based on these considerations, in the present invention we disclose KLRG2 as a protein without previous known association with tumor class under investigation and preferably used as a marker for breast tumor, and in general for cancers of this type. As described below, an antibody generated towards KLRG2 protein shows a selective immunoreactivity in histological preparation of breast cancer tissues, which indicates the presence of this protein in this cancer type. In particular our immunoistochemistry analysis indicates that the protein shows plasma membrane localization in tumor cells. Moreover, localization analysis of tumor cell lines showed that the proteins is exposed on the cell surface and accessible to the binding of specific antibodies. Finally, silencing of KLRG2 significantly reduced the invasiveness and proliferation properties of breast tumor cells lines. Based on the above evidences, KLRG2 is a likely target for the development of anti-cancer therapies being exposed to the action of affinity ligands and being involved in cellular processes relevant for tumor development.

A further aspect of this invention is a method of screening a tissue sample for malignancy, which comprises determining the presence in said sample of at least one of the above-mentioned tumor markers. This method includes detecting either the marker protein, e.g. by means of labeled monoclonal or polyclonal antibodies that specifically bind to the target protein, or the respective mRNA, e.g. by means of polymerase chain reaction techniques such as RT-PCR. The methods for detecting proteins in a tissue sample are known to one skilled in the art and include immunoradiometric, immunoenzymatic or immunohistochemical techniques, such as radioimmunoassays, immunofluorescent assays or enzyme-linked immunoassays. Other known protein analysis techniques, such as polyacrylamide gel electrophoresis (PAGE), Western blot or Dot blot are suitable as well. Preferably, the detection of the protein marker is carried out with the immune-histochemistry technology, particularly by means of High Through-Put methods that allow the analyses of the antibody immune-reactivity simultaneously on different tissue samples immobilized on a microscope slide. Briefly, each Tissue Micro Array (TMA) slide includes tissue samples suspected of malignancy taken from different patients, and an equal number of normal tissue samples from the same patients as controls. The direct comparison of samples by qualitative or quantitative measurement, e.g. by enzimatic or colorimetric reactions, allows the identification of tumors.

In one embodiment, the invention provides a method of screening a sample of breast tissue for malignancy, which comprises determining the presence in said sample of the C6orf98, C9orf46, FLJ37107, YIPF2, UNQ6126, TRYX3, DPY19L3, SLC39A10, C14orf135, DENND1B, EMID1, ERMP1, CRISP3 and KLRG2 protein tumor marker, variants or isoforms thereof as described above.

A further aspect of the invention is a method in vitro for determining the presence of a breast tumor in a subject, which comprises the steps of:
providing a sample of the tissue suspected of containing tumor cells;
determining the presence of a tumor marker as above defined, or a combination thereof in said tissue sample by detecting the expression of the marker protein or the presence of the respective mRNA transcript;

wherein the detection of one or more tumor markers in the tissue sample is indicative of the presence of tumor in said subject.

The methods and techniques for carrying out the assay are known to one skilled in the art and are preferably based on immunoreactions for detecting proteins and on PCR methods for the detection of mRNAs. The same methods for detecting proteins or mRNAs from a tissue sample as disclosed above can be applied.

A further aspect of this invention is the use of the tumor markers herein provided as targets for the identification of candidate antitumor agents. Accordingly, the invention provides a method for screening a test compound which comprises contacting the cells expressing a tumor-associated protein selected from: Chromosome 6 open reading frame 98 (C6orf98); Chromosome 9 open reading frame 46 (C9orf46); Putative uncharacterized protein (FLJ37107); Yip1 domain family, member 2 (YIPF2); Uncharacterized protein UNQ6126/PRO20091 (UNQ6126); Trypsin-X3 Precursor (TRYX3); DPY-19-like 3 (DPY19L3); Solute carrier family 39 (zinc transporter), member 10 (SLC39A10); Chromosome 14 open reading frame 135 (C14orf135); DENN/MADD domain containing 1B (DENND1B), EMI domain-containing protein 1 Precursor (EMID1) Endoplasmic reticulum metallopeptidase 1 (ERMP1), Cysteine-rich secretory protein 3 Precursor (CRISP3) and Killer cell lectin-like receptor subfamily G member 2 (KLRG2).

with the test compound, and determining the binding of said compound to said tumor-associated protein. In addition, the ability of the test compound to modulate the activity of each target molecule can be assayed.

A further aspect of the invention is an antibody or a fragment thereof, which is able to specifically recognize and bind to one of the tumor-associated proteins described above. The term “antibody” as used herein refers to all types of immunoglobulins, including IgG, IgM, IgA, IgD and IgE. Such antibodies may include polyclonal, monoclonal, chimeric, single chain, antibodies or fragments such as Fab or scFv. The antibodies may be of various origin, including human, mouse, rat, rabbit and horse, or chimeric antibodies. The production of antibodies is well known in the art. For the production of antibodies in experimental animals, various hosts including goats, rabbits, rats, mice, and others, may be immunized by injection with polypeptides of the present invention or any fragment or oligopeptide or derivative thereof which has immunogenic properties or forms a suitable epitope. Monoclonal antibodies may be produced following the procedures described in Kohler and Milstein, Nature 265:495 (1975) or other techniques known in the art.

The antibodies to the tumor markers of the invention can be used to detect the presence of the marker in histologic preparations or to distinguish tumor cells from normal cells. To that purpose, the antibodies may be labeled with radiocative, fluorescent or enzyme labels.

In addition, the antibodies can be used for treating proliferative diseases by modulating, e.g. inhibiting or abolishing the activity of a target protein according to the invention. Therefore, in a further aspect the invention provides the use of antibodies to a tumor-associated protein selected from: Chromosome 6 open reading frame 98 (C6orf98); Chromosome 9 open reading frame 46 (C9orf46); Putative uncharacterized protein (F1137107); Yip1 domain family, member 2 (YIPF2); Uncharacterized protein UNQ6126/PRO20091 (UNQ6126); Trypsin-X3 Precursor (TRYX3); DPY-19-like 3 (DPY19L3); Solute carrier family 39 (zinc transporter), member 10 (SLC39A10); Chromosome 14 open reading frame 135 (C14orf135); DENN/MADD domain containing 1B (DENND1B), EMI domain-containing protein 1 Precursor (EMID1), Endoplasmic reticulum metallopeptidase 1 (ERMP1) Cysteine-rich secretory protein 3 Precursor (CRISP3) and Killer cell lectin-like receptor subfamily G member 2 (KLRG2).

for the preparation of a therapeutic agent for the treatment of proliferative diseases. For use in therapy, the antibodies can be formulated with suitable carriers and excipients, optionally with the addition of adjuvants to enhance their effects.

A further aspect of the invention relates to a diagnostic kit containing suitable means for detection, in particular the polypeptides or polynucleotides, antibodies or fragments or derivatives thereof described above, reagents, buffers, solutions and materials needed for setting up and carrying out the immunoassays, nucleic acid hybridization or PCR assays described above.

Parts of the kit of the invention can be packaged individually in vials or bottles or in combination in containers or multicontainer units.

### DESCRIPTION OF THE FIGURES

FIG. 1. Analysis of Purified C6orf98 Recombinant Protein

Left panel: Comassie staining of purified His-tag C6orf98 fusion protein separated by SDS-PAGE; Right panel: WB on the purified recombinant protein stained with anti-C6orf98 antibody. Arrow marks the protein band of the expected size. Molecular weight markers are reported on the left.

FIG. 2. Staining of Breast Tumor TMA with anti-C6orf98 Antibodies

Examples of TMA of tumor (lower panel) and normal tissue samples (upper panel) stained with anti-C6orf98 antibodies. The antibody-stains specifically tumor cells (in dark gray);

FIG. 3. Analysis of Purified C9orf46 Recombinant Protein

Left panel: Comassie staining of purified His-tag C9orf46 fusion protein separated by SDS-PAGE; Right panel: WB on the C9orf46 protein stained with anti-C9orf46 antibody. Arrow marks the protein band of the expected size. Molecular weight markers are reported on the left.

FIG. 4. Staining of Breast Tumor TMA with Anti-C9orf46 Antibodies

Examples of TMA of tumor (lower panel) and normal tissue samples (upper panel) stained with anti-C9orf46 antibodies. The antibody stains specifically tumor cells (in dark gray).

FIG. 5. Expression of C9orf46 in Breast Tumor Cell Lines and Tissue Homogenates

Western blot analysis of C9orf46 expression in total protein extracts from: A) BT549 (line 1) and MCF-7 (line-2) breast tumor cells (corresponding to 2×10⁵ cells); B) HeLa cells (corresponding to 2×10⁵ cells) transfected with the empty pcDNA3 vector (lane 1) or with the plasmid construct encoding the C9orf46 gene (lane 2); C) Normal (lane 1=Pt#1; lane 2=Pt#2) or cancerous breast tissues from patients (lane 3=Pt#1; lane 4=Pt#2); stained with anti-C9orf46 antibody. Arrow marks the expected C9orf46 band. Molecular weight markers are reported on the left.

FIG. 6. Analysis of Purified FLJ37107 Recombinant Protein

Left panel: Comassie staining of purified His-tag FLJ37107 fusion protein separated by SDS-PAGE; Right panel: WB on the recombinant FLJ37107 protein stained with anti-FLJ37107 antibody. Arrow marks the protein band of the expected size. Molecular weight markers are reported on the left.

FIG. 7. Staining of Breast Tumor TMA with Anti-FLJ37107 Antibodies

Examples of TMA of tumor (lower panel) and normal tissue samples (upper panel) stained with anti-FLJ37107 antibodies. The antibody stains specifically tumor cells (in dark gray).

FIG. 8. Analysis of Purified YIPF2 Recombinant Protein

Left panel: Comassie staining of purified His-tag YIPF2 fusion protein separated by SDS-PAGE; Right panel: WB on the purified protein stained with anti-YIPF2 antibody. Arrow marks the protein band of the expected size.

Molecular weight markers are reported on the left.

FIG. 9. Staining of Breast Tumor TMA with Anti-YIPF2 Antibodies

Examples of TMA of tumor (lower panel) and normal tissue samples (upper panel) stained with anti-YIPF2 antibodies. The antibody stains specifically tumor cells (in dark gray).

FIG. 10. Analysis of Purified UNQ6126 Recombinant Protein

Left panel: Comassie staining of purified His-tag UNQ6126 fusion protein separated by SDS-PAGE; Right panel: WB on the purified recombinant protein stained with anti-UNQ6126 antibody. Arrow marks the protein band of the expected size. Molecular weight markers are reported on the left.

FIG. 11. Staining of Breast Tumor TMA with Anti-UNQ6126 Antibodies

Examples of TMA of tumor (lower panel) and normal tissue samples (upper panel) stained with anti-UNQ6126 antibodies. The antibody stains specifically tumor cells (in dark gray).

FIG. 12. Analysis of Purified TRYX3 Recombinant Protein

Left panel: Comassie staining of purified His-tag TRYX3 fusion protein separated by SDS-PAGE; Right panel: WB on the purified recombinant protein stained with anti-TRYX3 antibody. Arrow marks the protein band of the expected size. Molecular weight markers are reported on the left.

FIG. 13. Staining of Breast Tumor TMA with Anti-TRYX3 Antibodies

Examples of TMA of tumor (lower panel) and normal tissue samples (upper panel) stained with anti-TRYX3 antibodies. The antibody stains specifically tumor cells (in dark gray).

FIG. 14. Analysis of Purified DPY19L3 Recombinant Protein

Left panel: Comassie staining of purified His-tag DPY19L3 fusion protein separated by SDS-PAGE; Right panel: WB on the purified protein stained with anti-DPY19L3 antibody. Arrow marks the protein band of the expected size. Molecular weight markers are reported on the left.

FIG. 15. Staining of Breast Tumor TMA with Anti-DPY19L3 Antibodies

Examples of TMA of tumor (lower panel) and normal tissue samples (upper panel) stained with anti-DPY19L3 antibodies. The antibody stains specifically tumor cells (in dark gray).

FIG. 16. Expression and Localization of DPY19L3 in Tumor Cell Lines

Panel A: Western blot analysis of DPY19L3 expression in total protein extracts separated by SDS-PAGE from: MCF-7 (lane 1), MDA-MB231 (lane 2), SKBR-3 (lane 3), breast derived tumor cells; Arrow marks the protein band of the expected size.

Molecular weight markers are reported on the left.

Panel B: Flow cytometry analysis of DPY19L3 cell surface localization in MCF-7 and SKBR-3 cells stained with a negative control antibody (filled curve) or with anti-DPY19L3 antibody (empty curve). X axis, Fluorescence scale; Y axis, Cells (expressed as % relatively to major peaks).

FIG. 17. Analysis of Purified SLC39A10 Recombinant Protein

Left panel: Comassie staining of purified His-tag SLC39A10 protein separated by SDS-PAGE; Right panel: WB on the recombinant protein stained with anti-SLC39A10 antibody. The low molecular weight bands correspond to partially degraded forms of SLC39A10 protein. Molecular weight markers are reported on the left.

FIG. 18. Staining of Breast Tumor TMA with anti-SLC39A10 Antibodies

Examples of TMA of tumor (lower panel) and normal tissue samples (upper panel) stained with anti-SLC39A10 antibodies. The antibody stains specifically tumor cells (in dark gray).

FIG. 19. Confocal Microscopy Analysis of Expression and Localization of SLC39A10

HeLa cells transfected with the empty pcDNA3 vector (upper panels) or with the plasmid construct encoding the SLC39A10 gene (lower panels) stained with secondary antibodies (left panels) and with anti-SLC39A10 antibodies (right panels). Arrowheads mark surface specific localization.

FIG. 20. Expression and Localization of SLC39A10 in Breast Tumor Cells

Flow cytometry analysis of SLC39A10 cell surface localization SKBR3 tumor cells stained with a negative control antibody (filled curve or with anti-SLC39A10 antibody (empty curve). X axis, Fluorescence scale; Y axis, Cells (expressed as percentage relatively to major peaks).

FIG. 21. Analysis of Purified C14orf135 Recombinant Protein

Left panel: Comassie staining of purified His-tag C14orf135 fusion protein espressed in E. coli separated by SDS-PAGE; Right panel: WB on the recombinant protein stained with anti-C14orf135 antibody. Arrow marks the protein band of the expected size. Molecular weight markers are reported on the left.

FIG. 22. Staining of Breast Tumor TMA with Anti-C14orf135 Antibodies

Examples of TMA of tumor (lower panel) and normal tissue samples (upper panel) stained with anti-C14orf135 antibodies. The antibody stains specifically tumor cells and their secretion products (in dark gray). Moreover antibody stain also accumulated at the plasma membrane of tumor cells (boxed image, marker by arrows).

FIG. 23. Analysis of purified DENND1B recombinant protein

Left panel: Comassie staining of purified His-tag DENND1B fusion protein separated by SDS-PAGE; Right panel: WB on the purified DENND1B protein stained with anti-DENND1B antibody. Arrow marks the protein band of the expected size. Molecular weight markers are reported on the left.

FIG. 24. Staining of Breast Tumor TMA with Anti-DENND1B Antibodies

Examples of TMA of tumor (lower panel) and normal tissue samples (upper panel) stained with anti-DENND1B antibodies. The antibody stains specifically tumor cells (in dark gray).

FIG. 25. Analysis of Purified EMID1 Recombinant Protein

Left panel: Comassie staining of purified His-tag EMID1 fusion protein separated by SDS-PAGE; Right panel: WB on the recombinant protein stained with anti-EMID1 antibody. Arrow marks the protein band of the expected size. The high molecular weight bands are consistent with multimers of the protein as defined by Mass spectromic analysis. Molecular weight markers are reported on the left.

FIG. 26. Staining of Breast Tumor TMA with Anti-EMID1 Antibodies

Examples of TMA of tumor (lower panel) and normal tissue samples (upper panel) stained with anti-EMID1 antibodies. The antibody specifically stains secretion products of tumor cells (in dark gray).

FIG. 27. Analysis of Purified ERMP1 Recombinant Protein

Left panel: Comassie staining of purified His-tag ERMP1 fusion protein separated by SDS-PAGE; Right panel: WB on the recombinant protein stained with anti-ERMP1 antibody. Arrow marks the protein band of the expected size. Molecular weight markers are reported on the left.

FIG. 28. Staining of Breast Tumor TMA with Anti-ERMP1 Antibodies

Examples of TMA of tumor (lower panel) and normal tissue samples (upper panel) stained with anti-ERMP1 antibodies. The antibody stains specifically tumor cells (in dark gray).

FIG. 29. Expression and Localization of ERMP1 in Tumor Cell Lines

Panel A:

Western blot analysis of ERMP1 expression in total protein extracts separated by SDS-PAGE from HEK-293T cells (corresponding to 1×10⁶ cells) transfected with the empty pcDNA3 vector (lane 1) or with the plasmid construct encoding the ERMP1 gene (lane 2);

Panel B:

Western blot analysis of ERMP1 expression in total protein extracts separated by SDS-PAGE from MCF-7 (lane 1) and SKBR-3 (lane 2) tumor cells (corresponding to 2×10⁵ cells). Arrow marks the expected ERMP1 band. Molecular weight markers are reported on the left.

Panel C:

Flow cytometry analysis of ERMP1 cell surface localization in SKBR-3 tumor cells stained with a negative control antibody (filled curve or with anti-ERMP1 antibody (empty curve). X axis, Fluorescence scale; Y axis, Cells (expressed as % relatively to major peaks).

FIG. 30. ERMP1 confers malignant cell phenotype—The proliferation and the invasiveness properties of the MCF7 cell line were assessed after transfection with ERMP1-siRNA and a scramble siRNA control using the MTT and the Boyden in vitro invasion assays, respectively.

Panel A.

Cell migration/invasiveness measured by the Boyden migration assay. The graph represents the reduced migration/invasiveness of MCF7 treated with the ERMP1-specific siRNA. Small boxes under the columns show the visual counting of the migrated cells.

Panel B.

Cell proliferation determined by the MTT incorporation assay. The graph represents the reduced proliferation of the MCF7 tumor cells upon treatment with ERMP1-siRNA, as determined by spectrophotometric reading.

FIG. 31. Analysis of Purified CRISP3 Recombinant Protein

Left panel: Comassie staining of purified His-tag CRISP3 fusion protein separated by SDS-PAGE; Right panel: WB on the purified recombinant CRISP3 protein stained with anti-CRISP3 antibody. Arrow marks the protein band of the expected size. The high molecular weight bands are consistent with protein dimersas defined by Mass spectromic analysis. Molecular weight markers are reported on the left.

FIG. 32. Staining of Breast Tumor TMA with Anti-CRISP3 Antibodies

Examples of TMA of breast tumor (lower panel) and normal tissue samples (upper panel) stained with anti-CRISP3 antibodies. The antibody stains specifically tumor cells (in dark gray).

FIG. 33. Analysis of Purified KLRG2 Recombinant Protein Expressed in E. coli

Left panel: Comassie staining of purified His-tag KLRG2 fusion protein expressed in E. coli separated by SDS-PAGE; Right panel: WB on the purified recombinant protein stained with anti-KLRG2 antibody. Arrow marks the protein band of the expected size. Molecular weight markers are reported on the left.

FIG. 34. Staining of breast tumor TMA with anti-KLRG2 antibodies. Examples of TMA of tumor (lower panel) and normal tissue samples (upper panel) stained with anti-KLRG2 antibodies. The antibody-stains specifically tumor cells (in dark gray).

FIG. 35. Expression and Localization of KLRG2 in Tumor Cell Lines

Panel A:

Western blot analysis of KLRG2 expression in total protein extracts separated by SDS-PAGE from HeLa cells (corresponding to 1×10⁶ cells) transfected with the empty pcDNA3 vector (lane 1), with the plasmid construct encoding the isoform 2 of the KLRG2 gene (lane 2); or with the plasmid construct encoding the isoformal of the KLRG2 gene (lane 3); Arrows mark the expected KLRG2 bands.

Panel B:

Western blot analysis of KLRG2 expression in total protein extracts separated by SDS-PAGE from normal breast tissues (1=Pt#1; 2=Pt#2; 3=Pt#3; 4=Pt#4) or from breast cancer tissues 5=Pt#1; 6=Pt#2; 7=Pt#3; 8=Pt#4); stained with anti-KLRG2 antibody. Arrow marks one of the expected KLRG2 band. Molecular weight markers are reported on the right.

Panel C:

Flow cytometry analysis of KLRG2 cell surface localization in SKBR-3 cells stained with a negative control antibody (filled curve or with anti-KLRG2 antibody (empty curve). X axis, Fluorescence scale; Y axis, Cells (expressed as % relatively to major peaks).

FIG. 36. KLRG2 Confer Malignant Cell Phenotypes

The proliferation and the migration/invasiveness phenotypes of MCF7 cell line were assessed after transfection with KLRG2-siRNA and a scramble siRNA control using the MTT and the Boyden in vitro invasion assay, respectively.

Panel A.

Cell migration/invasiveness measured by the Boyden migration assay. The graph represents the reduced migration/invasiveness of MCF7 treated with the KLRG2 specific siRNA. Small boxes under the columns show the visual counting of the migrated cells.

Panel B.

Cell proliferation determined by the MTT incorporation assay. The graph represents the reduced proliferation of the MCF7 tumor cells upon treatment with KLRG2-siRNA, as determined by spectrophotometric reading.

The following examples further illustrate the invention.

### EXAMPLES

### Example 1

### Generation of Recombinant Human Protein Antigens and Antibodies to Identify Tumor Markers

Methods

The entire coding region or suitable fragments of the genes encoding the target proteins, were designed for cloning and expression using bioinformatic tools with the human genome sequence as template (Lindskog M et al (2005). Where present, the leader sequence for secretion was replaced with the ATG codon to drive the expression of the recombinant proteins in the cytoplasm of E. coli. For cloning, genes were PCR-amplified from templates derived from Mammalian Gene Collection (http://mgc.nci.nih.gov/) clones using specific primers. Clonings were designed so as to fuse a 10 histidine tag sequence at the 5′ end, annealed to in house developed vectors, derivatives of vector pSP73 (Promega) adapted for the T4 ligation independent cloning method (Nucleic Acids Res. 1990 October 25; 18(20): 6069-6074) and used to transform E. coli NovaBlue cells recipient strain. E. coli tranformants were plated onto selective LB plates containing 100 μg/ml ampicillin (LB Amp) and positive E. coli clones were identified by restriction enzyme analysis of purified plasmid followed by DNA sequence analysis. For expression, plasmids were used to transform BL21-(DE3) E. coli cells and BL21-(DE3) E. coli cells harbouring the plasmid were inoculated in ZYP-5052 growth medium (Studier, 2005) and grown at 37° C. for 24 hours. Afterwards, bacteria were collected by centrifugation, lysed into B-Per Reagent containing 1 mM MgCl2, 100 units DNAse I (Sigma), and 1 mg/ml lysozime (Sigma). After 30 min at room temperature under gentle shaking, the lysate was clarified by centrifugation at 30.000 g for 40 min at 4° C. All proteins were purified from the inclusion bodies by resuspending the pellet coming from lysate centrifugation in 40 mM TRIS-HCl, 1 mM TCEP {Tris(2-carboxyethyl)-phosphine hydrochloride, Pierce} and 6M guanidine hydrochloride, pH 8 and performing an IMAC in denaturing conditions. Briefly, the resuspended material was clarified by centrifugation at 30.000 g for 30 min and the supernatant was loaded on 0.5 ml columns of Ni-activated Chelating Sepharose Fast Flow (Pharmacia). The column was washed with 50 mM TRIS-HCl buffer, 1 mM TCEP, 6M urea, 60 mM imidazole, 0.5M NaCl, pH 8. Recombinant proteins were eluted with the same buffer containing 500 mM imidazole. Proteins were analysed by SDS-Page and their concentration was determined by Bradford assay using the BIORAD reagent (BIORAD) with a bovine serum albumin standard according to the manufacturer's recommendations. The identity of recombinant affinity purified proteins was further confirmed by mass spectrometry (MALDI-TOF), using standard procedures.

To generate antisera, the purified proteins were used to immunize CD1 mice (6 week-old females, Charles River laboratories, 5 mice per group) intraperitoneally, with 3 protein doses of 20 micrograms each, at 2 week-interval. Freund's complete adjuvant was used for the first immunization, while Freund's incomplete adjuvant was used for the two booster doses. Two weeks after the last immunization animals were bled and sera collected from each animal was pooled.

Results

Gene fragments of the expected size were obtained by PCR from specific clones of the Mammalian Gene Collection or, alternatively, from cDNA generated from pools of total RNA derived from Human testis, Human placenta, Human bone marrow, Human fetal brain, using primers specific for each gene.

For the C6orf98 gene, a fragment corresponding to nucleotides 67 to 396 of the transcript ENST00000409023 and encoding a protein of 110 residues, corresponding to the amino acid region from 22 to 132 of ENSP00000386324 sequence was obtained.

For the C9orf46 gene, a fragment corresponding to nucleotides 439 to 663 of the transcript ENST00000107020 and encoding a protein of 75 residues, corresponding to the amino acid region from 73 to 147 of ENSP00000223864 sequence was obtained.

For the F1137107 gene, a fragment corresponding to nucleotides 661-972 of the transcript gi|58218993|ref|NM_{—}001010882.1 and encoding a protein of 104 residues, corresponding to the amino acid region from 1 to 104 of gi|58218994|ref|NP_{—}001010882.1 sequence was obtained.

For the YIPF2 gene, a fragment corresponding to nucleotides 107 to 478 of the transcript ENST00000393508 and encoding a protein of 124 residues, corresponding to the amino acid region from 1 to 124 of ENSP00000377144 sequence was obtained.

For the UNQ6126 gene, a fragment corresponding to a fragment corresponding to nucleotides 88 to 471 of the transcript gi|169216088|ref|XM_{—}001719570.1| and encoding a protein of 128 residues, and encoding an amino acid region from 30 to 147 of sp|Q6UXV3|YV010 sequence was obtained.

For the TRYX3 gene, a fragment corresponding to nucleotides 230 to 781 of the transcript ENST00000304182 and encoding a protein of 184 residues, corresponding to the amino acid region from 41 to 224 of ENSP00000307206 sequence was obtained.

For the DPY19L3 gene, a fragment corresponding to nucleotides 158 to 463 of the transcript ENST00000392250 and encoding a protein of 102 residues, corresponding to the amino acid region from 1 to 102 of ENSP00000376081 sequence was obtained.

For the SLC39A10 gene, a DNA fragment corresponding to nucleotides 154-1287 of the transcript ENST00000359634 and encoding a protein of 378 residues, corresponding to the amino acid region from 26 to 403 of ENSP00000352656 sequence was obtained.

For the C14orf135 gene, a fragment corresponding to nucleotides 2944 to 3336 of the transcript ENST00000317623 and encoding a protein of 131 residues, corresponding to the amino acid region 413 to 543 of ENSP00000317396 sequence was obtained.

For the DENND1B gene, a fragment corresponding to nucleotides 563 to 1468 of the transcript ENST00000235453 and encoding a protein of 302 residues, corresponding to the amino acid region from 95 to 396 of ENSP00000235453 sequence was obtained.

For the EMID1 gene, a fragment corresponding to nucleotides 203-670 of the transcript OTTHUMT00000075712 and encoding a protein of 156 residues, corresponding to the amino acid region from 33 to 188 of OTTHUMP00000028901 sequence was obtained.

For the ERMP1 gene, a fragment corresponding to nucleotides 55-666 of the transcript ENST00000339450 and encoding a protein of 204 residues, corresponding to the amino acid region from 1 to 204 of ENSP00000340427 sequence was obtained.

For the CRISP3 gene, a fragment corresponding to nucleotides 62-742 of the transcript ENST00000393666 and encoding a protein of 227 residues, corresponding to the amino acid region from 19 to 245 of ENSP0000037727 sequence was obtained.

For the KLRG2 gene, a fragment corresponding to nucleotides 70 to 849 of the transcript ENST00000340940 and encoding a protein of 260 residues, corresponding to the amino acid region from 1 to 260 of ENSP00000339356 sequence was obtained.

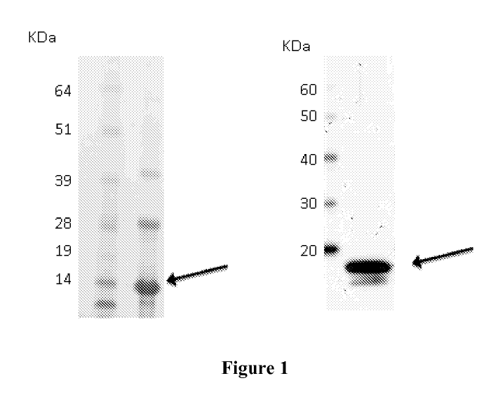

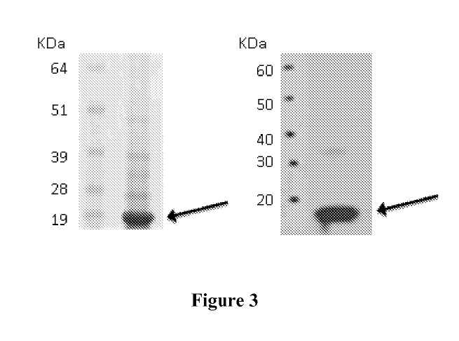

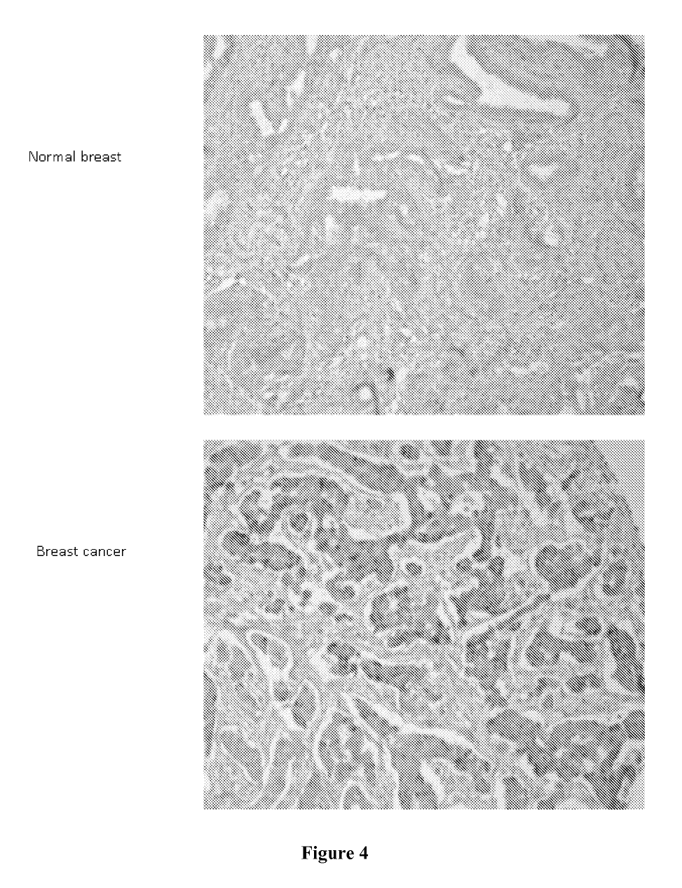

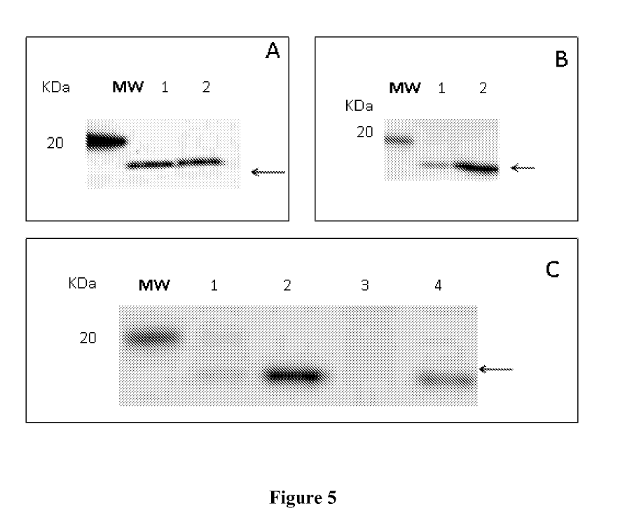

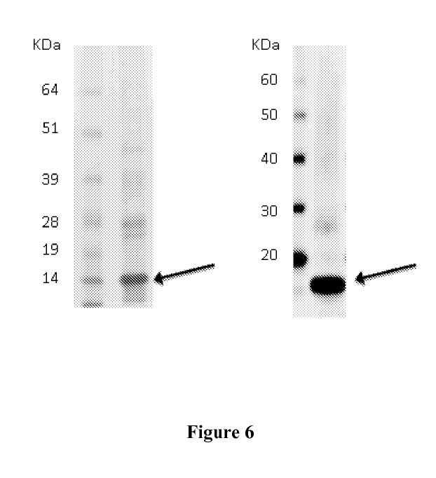

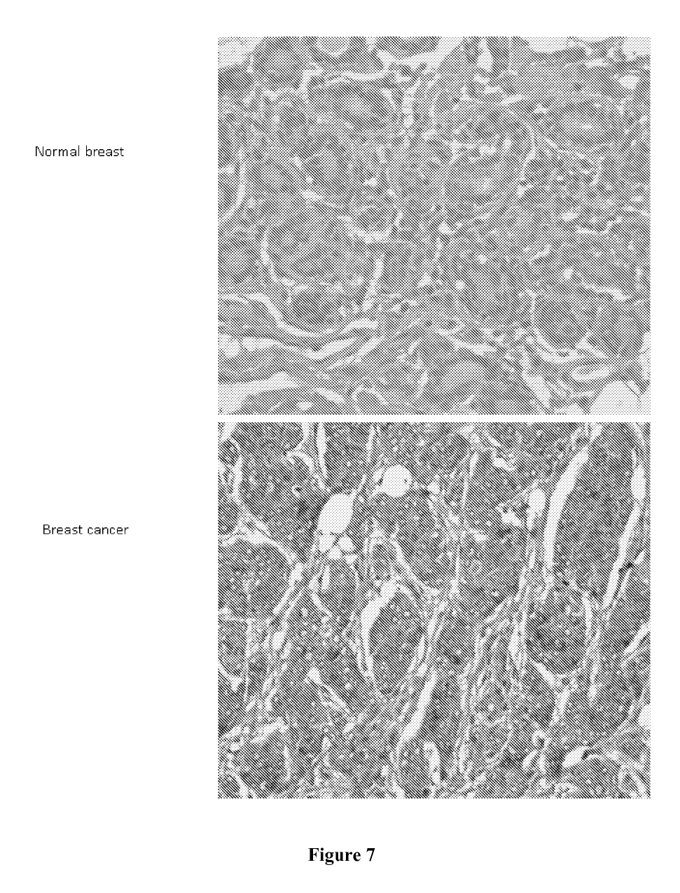

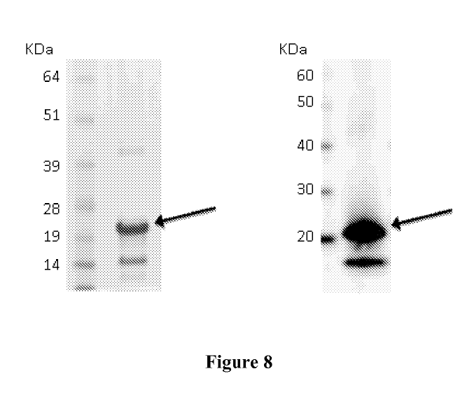

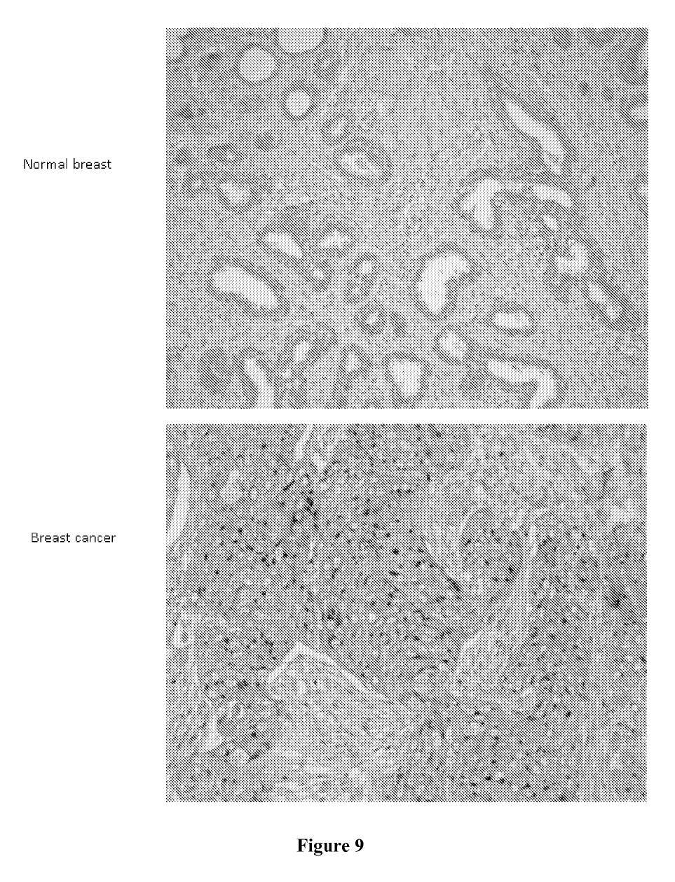

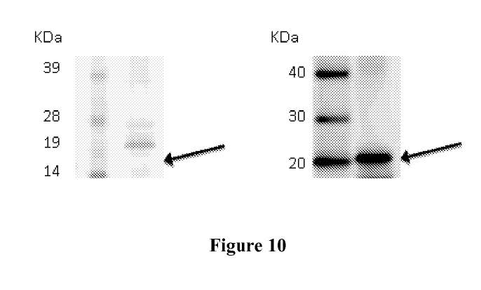

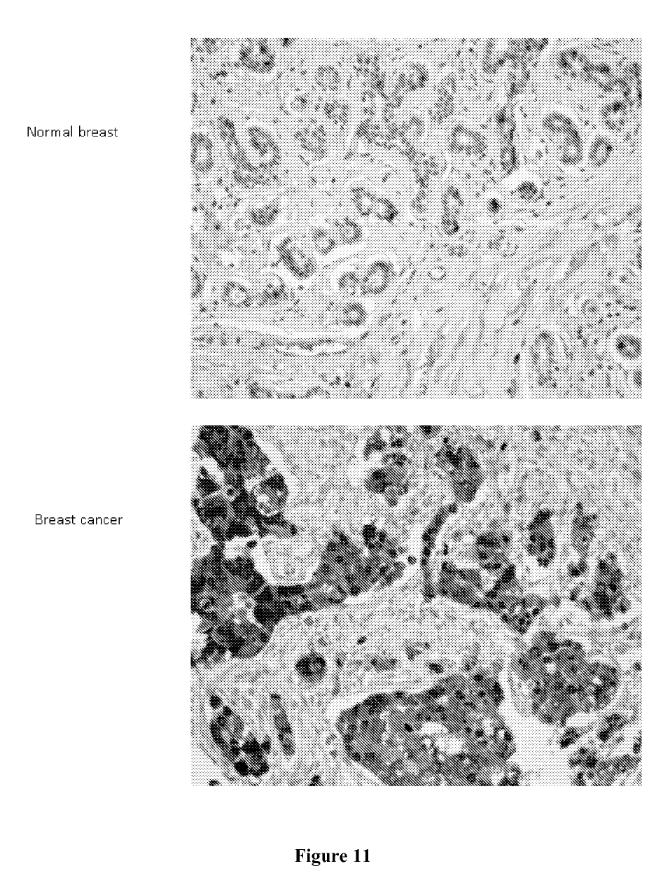

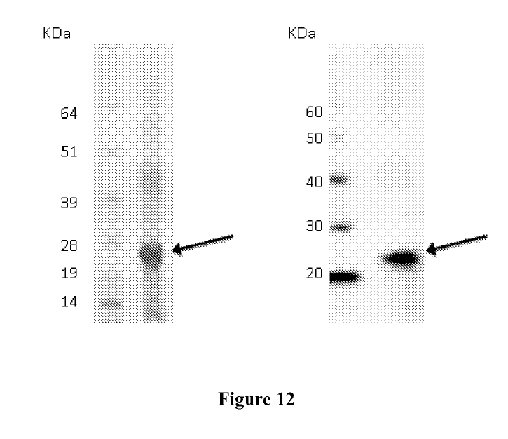

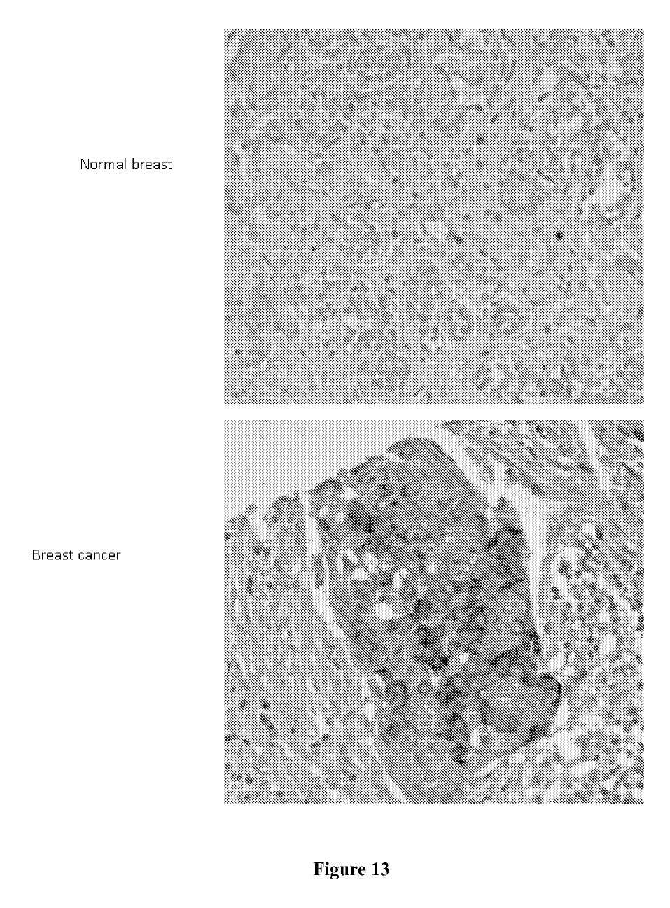

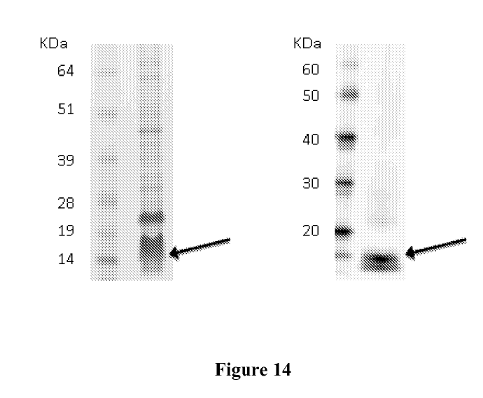

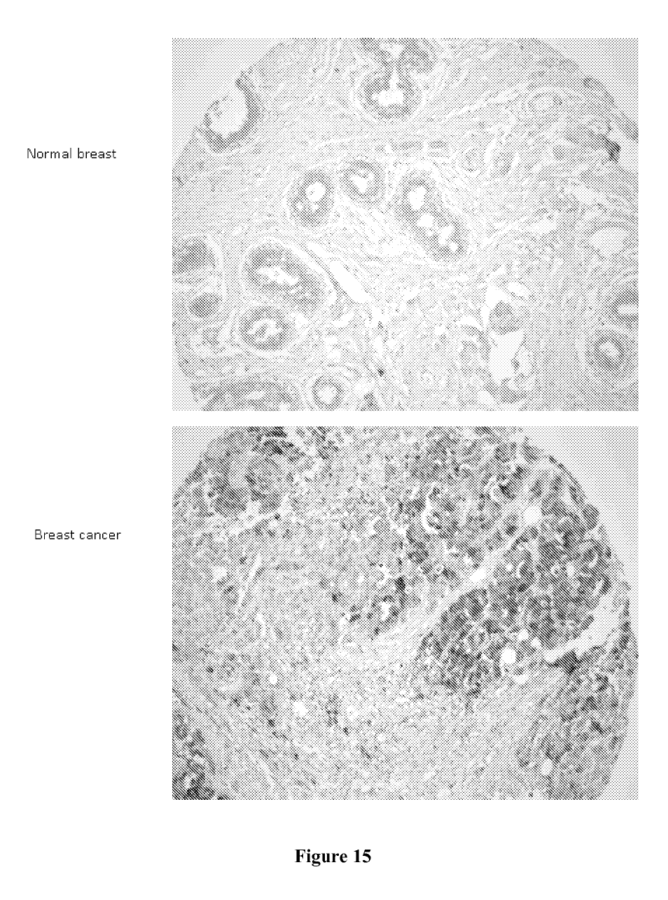

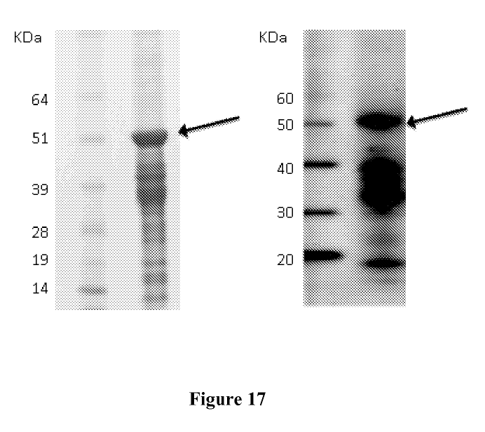

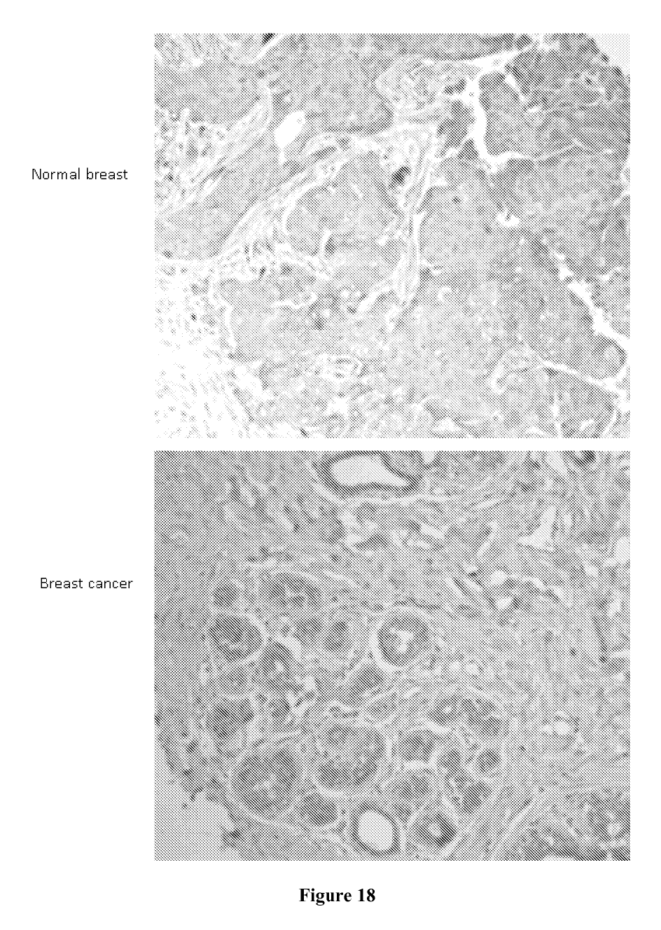

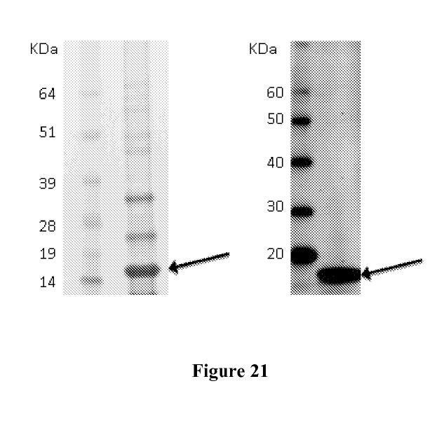

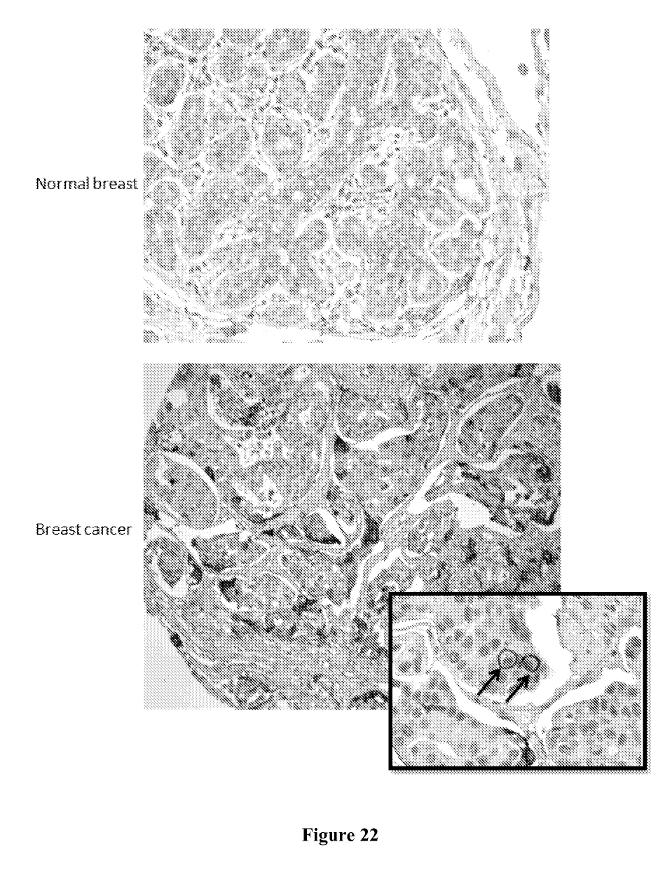

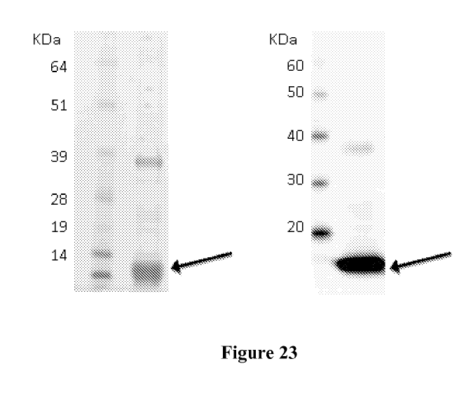

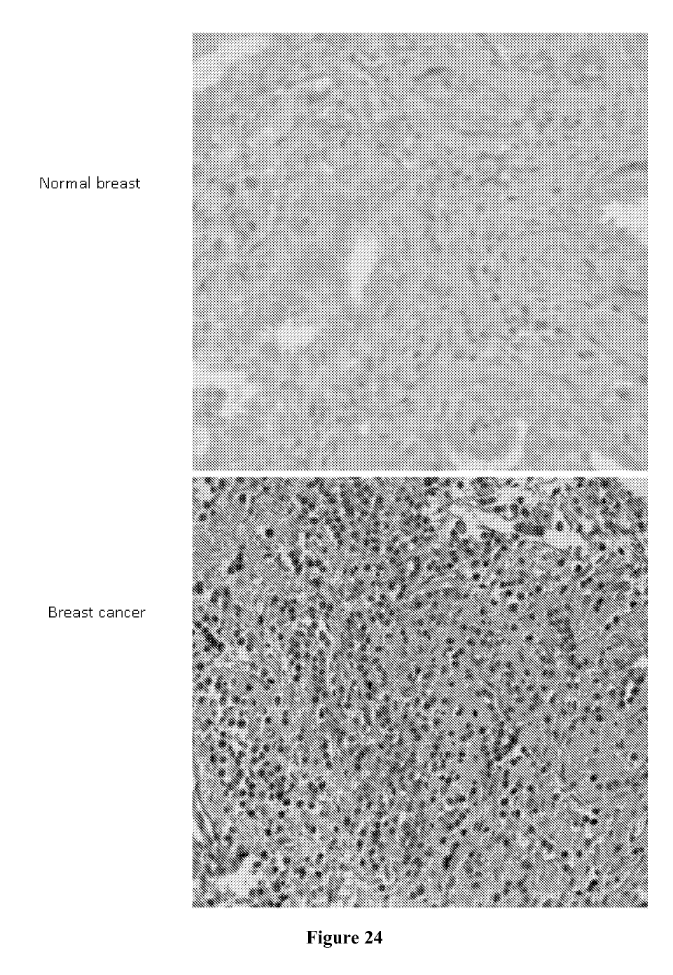

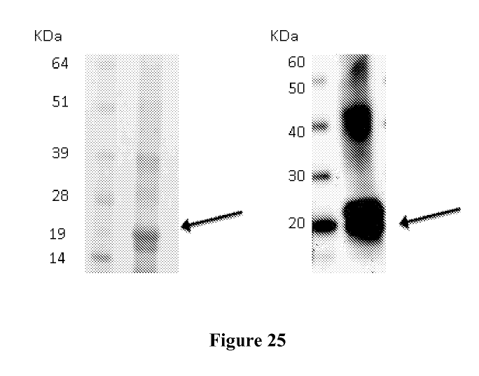

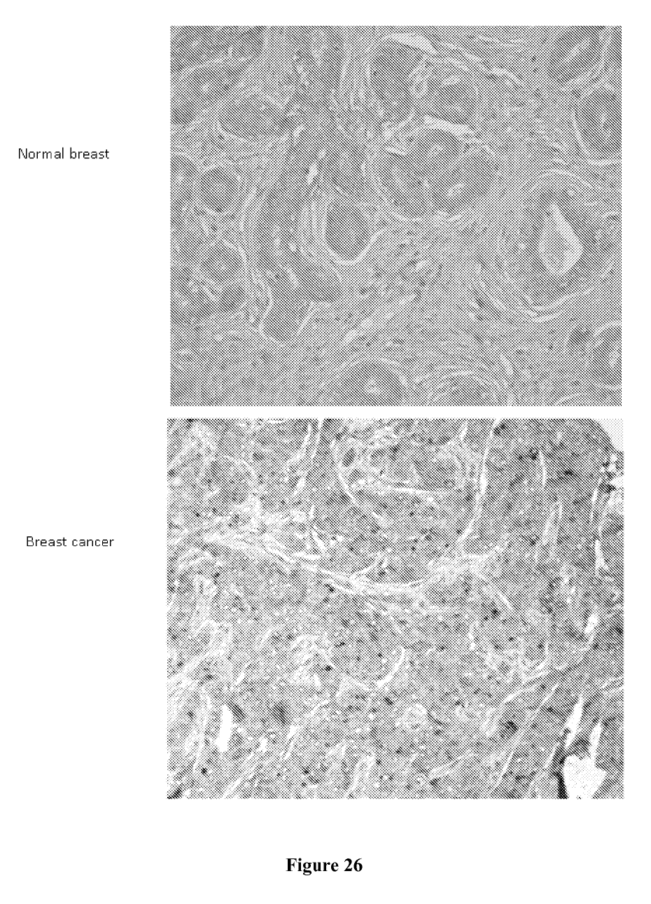

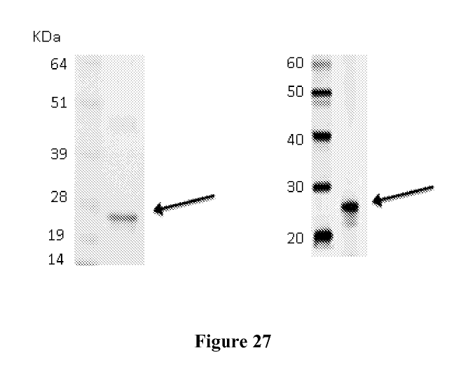

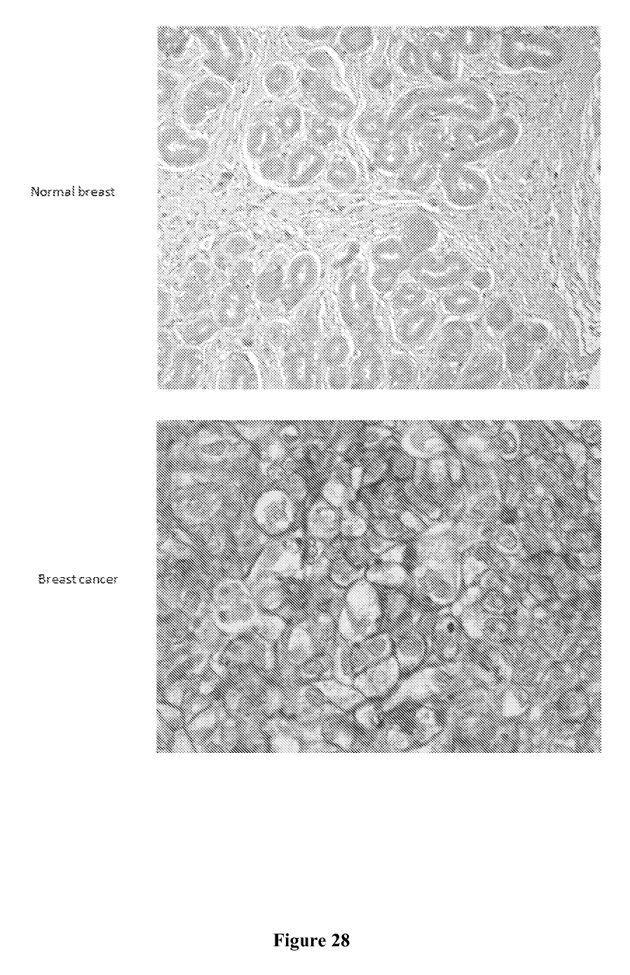

A clone encoding the correct amino acid sequence was identified for each gene/gene-fragment and, upon expression in E. coli, a protein of the correct size was produced and subsequently purified using affinity chromatography (FIGS. 1, 3, 6, 8, 10, 12, 14, 17, 21, 23, 25, 27, 31, 33 left panels). As shown in the figures, in some case SDS-PAGE analysis of affinity-purified recombinant proteins revealed the presence of extra bands, of either higher and/or lower masses. Mass spectrometry analysis confirmed that they corresponded to either aggregates or degradation products of the protein under analysis.

Antibodies generated by immunization specifically recognized their target proteins in Western blot (WB) (FIGS. 1, 3, 6, 8, 10, 12, 14, 17, 21, 23, 25, 27, 31, 33; right panels).

### Example 2

### Tissue Profiling by Immune-Histochemistry

Methods

The analysis of the antibodies' capability to recognize their target proteins in tumor samples was carried out by Tissue Micro Array (TMA), a miniaturized immuno-histochemistry technology suitable for HTP analysis that allows to analyse the antibody immuno-reactivity simultaneously on different tissue samples immobilized on a microscope slide.

Since the TMAs include both tumor and healthy tissues, the specificity of the antibodies for the tumors can be immediately appreciated. The use of this technology, differently from approaches based on transcription profile, has the important advantage of giving a first hand evaluation on the potential of the markers in clinics. Conversely, since mRNA levels not always correlate with protein levels (approx. 50% correlation), studies based on transcription profile do not provide solid information regarding the expression of protein markers.

A tissue microarray was prepared containing formalin-fixed paraffin-embedded cores of human tissues from patients affected by breast cancer and corresponding normal tissues as controls and analyzed using the specific antibody sample. In total, the TMA design consisted in 10 tumor breast tumor samples and 10 normal tissues from 5 well pedigreed patients (equal to two tumor samples and 2 normal tissues from each patient) to identify promising target molecules differentially expressed in cancer and normal cells. The direct comparison between tumor and normal tissues of each patient allowed the identification of antibodies that stain specifically tumor cells and provided indication of target expression in breast tumor.

To further confirm the association of each protein with breast tumors a tissue microarray was prepared containing 100 formalin-fixed paraffin-embedded cores of human breast tissues from 50 patients (equal to two tissue samples from each patient).

All formalin fixed, paraffin embedded tissues used as donor blocks for TMA production were selected from the archives at the IE0 (Istituto Europeo Oncologico, Milan). Corresponding whole tissue sections were examined to confirm diagnosis and tumour classification, and to select representative areas in donor blocks. Normal tissues were defined as microscopically normal (non-neoplastic) and were generally selected from specimens collected from the vicinity of surgically removed tumors. The TMA production was performed essentially as previously described (Kononen J et al (1998) Nature Med. 4:844-847; Kallioniemi O P et al (2001) Hum. MoI. Genet. 10:657-662). Briefly, a hole was made in the recipient TMA block. A cylindrical core tissue sample (1 mm in diameter) from the donor block was acquired and deposited in the recipient TMA block. This was repeated in an automated tissue arrayer “Galileo TMA CK 3500” (BioRep, Milan) until a complete TMA design was produced. TMA recipient blocks were baked at 42 <0>C for 2 h prior to sectioning. The TMA blocks were sectioned with 2-3 mm thicknes using a waterfall microtome (Leica), and placed onto poly-L-lysinated glass slides for immunohistochemical analysis. Automated immunohistochemistry was performed as previously described (Kampf C. et al (2004) Clin. Proteomics 1:285-300). In brief, the glass slides were incubated for 30′ min in 60° C., de-paraffinized in xylene (2×15 min) using the Bio-Clear solution (Midway. Scientific, Melbourne, Australia), and re-hydrated in graded alcohols. For antigen retrieval, slides were immersed 0.01 M Na-citrate buffer, pH 6.0 at 99° C. for 30 min Slides were placed in the Autostainer (R) (DakoCytomation) and endogenous peroxidase was initially blocked with 3% H2O2, for 5 min. Slides were then blocked in Dako Cytomation Wash Buffer containing 5% Bovine serum albumin (BSA) and subsequently incubated with mouse antibodies for 30′ (dilution 1:200 in Dako Real™ dilution buffer). After washing with DakoCytomation wash buffer, slides were incubated with the goat anti-mouse peroxidase conjugated Envision(R) for 30 min each at room temperature (DakoCytomation). Finally, diaminobenzidine (DakoCytomation) was used as chromogen and Harris hematoxylin (Sigma-Aldrich) was used for counterstaining. The slides were mounted with Pertex(R) (Histolab).

The staining results have been evaluated by a trained pathologist at the light microscope, and scored according to both the percentage of immunostained cells and the intensity of staining. The individual values and the combined score (from 0 to 300) were recorded in a custom-tailored database. Digital images of the immunocytochemical findings have been taken at a Leica DM LB light microscope, equipped with a Leica DFC289 color camera.

Results

A TMA design was obtained, representing tumor tissue samples and normal tissues, derived from patients affected by breast tumor. The results from tissue profiling showed that the antibodies specific for the recombinant proteins (see Example 1) are strongly immunoreactive on breast tumor cancer tissues, while no or poor reactivity was detected in normal tissues, indicating the presence of the target proteins in breast tumors. Based on this finding, the detection of target proteins in tissue samples can be associated with breast tumor. In some cases immunoreactivity accumulated at the cell membrane of tumor cells providing a first-hand indication on the surface localization of the target proteins.

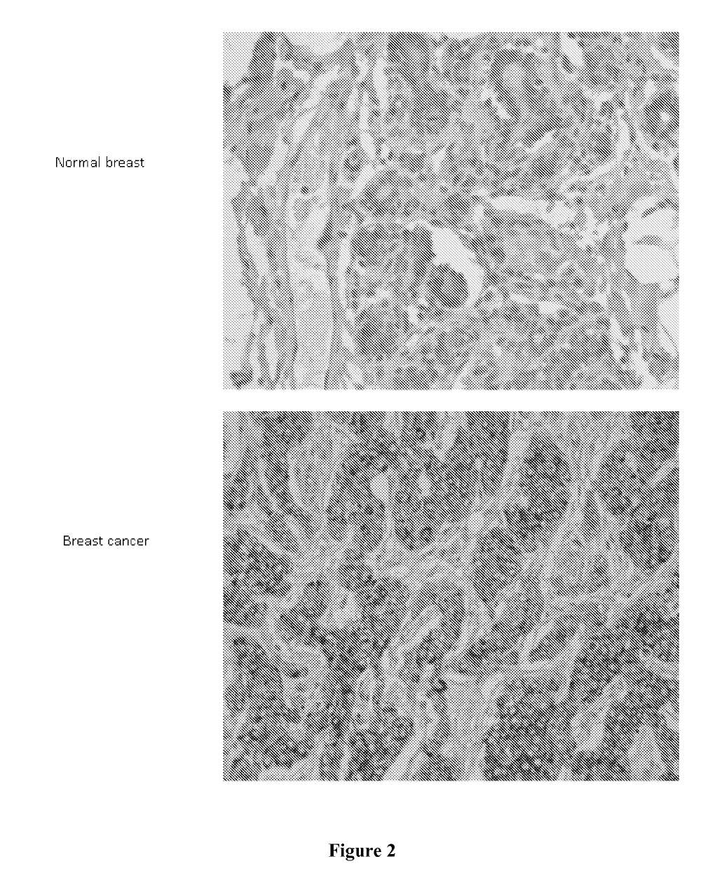

The capability of target-specific antibodies to stain breast tumor tissues is summarized in Table I. Representative examples of microscopic enlargements of tissue samples stained by each antibody are reported in FIGS. 2; 4; 7; 9; 11; 13; 15; 18; 22; 24; 26; 28; 32, 34).

Table reports the percentage of positive breast tumor tissue samples after staining with the target specific antibodies.

| | Percentage of Breast |
|---|---|
| | tumor tissues showing |
| Marker name | positive IHC staining |
| C6orf98 | 80 |
| C9orf46 | 20 |
| FLJ37107 | 60 |
| YIPF2 | 40 |
| UNQ6126 | 82 |
| TRYX3 | 40 |
| DPY19L3 | 83 |
| SLC39A10 | 27 |
| C14orf135 | 20* |
| DENND1B | 20 |
| EMID1 | 20* |
| ERMP1 | 45** |
| CRISP3 | 40 |
| KLRG2 | 34** |

*The antibody stains both breast tumor cells and secretion products indicating that the corresponding proteins are specifically released by tumor cells.
**The antibody stains the cell membrane of tumor cells

### Example 3

### Expression and Localization of Target Protein in Transfected Mammalian Cells

Methods

The specificity of the antibodies for each target proteins was assessed by Western blot analysis on total protein extracts from eukaryotic cells transiently transfected with plasmid constructs containing the complete sequences of the genes encoding the target proteins. Where indicated, expression and localization of target proteins were investigated by confocal microscopy analysis of transfected cells. Examples of this type of experiments are represented for C9orf46 (corresponding to Transcript ID ENST00000223864), KLRG2 (cloned sequences corresponding to Transcripts ENST00000340940 and ENST00000393039, corresponding to two transcript variants), ERMP 1 (cloned sequence corresponding to Transcripts ENST00000339450), SLC39A10 (cloned sequence corresponding to Transcript ENST00000359634).

For clonings, cDNA were generated from pools of total RNA derived from Human testis, Human placenta, Human bone marrow, Human fetal brain, in reverse transcription reactions and the entire coding regions were PCR-amplified with specific primers pairs. PCR products were cloned into plasmid pcDNA3 (Invitrogen). HeLa or Hek-293T cells were grown in DMEM-10% FCS supplemented with 1 mM Glutamine were transiently transfected with preparation of the resulting plasmids and with the empty vector as negative control using the Lipofectamine-2000 transfection reagent (Invitrogen). After 48 hours, cells were collected and analysed by Western blot or confocal microscopy. For Western blot, cells were lysed with PBS buffer containing 1% Triton X100 and total cell extracts (corresponding to 2×10⁵ cells) were separated on pre-cast SDS-PAGE gradient gels (NuPage 4-12% Bis-Tris gel, Invitrogen) under reducing conditions, followed by electro-transfer to nitrocellulose membranes (Invitrogen) according to the manufacturer's recommendations. The membranes were blocked in blocking buffer composed of 1×PBS-0.1% Tween 20 (PBST) added with 10% dry milk, for 1 h at room temperature, incubated with the antibody diluted 1:2500 in blocking buffer containing 1% dry milk and washed in PBST-1%. The secondary HRP-conjugated antibody (goat anti-mouse immunoglobulin/HRP, Perkin Elmer) was diluted 1:5000 in blocking buffer and chemiluminescence detection was carried out using a Chemidoc-IT UVP CCD camera (UVP) and the Western Lightning™ cheminulescence Reagent Plus (Perkin Elmer), according to the manufacturer's protocol.

For confocal microscopy analysis, the cells were plated on glass cover slips and after 48 h were washed with PBS and fixed with 3% p-formaldheyde solution in PBS for 20 min at RT. For surface staining, cells were incubated overnight at 4° C. with polyclonal antibodies (1:200). The cells were then stained with Alexafluor 488-labeled goat anti-mouse antibodies (Molecular Probes). DAPI (Molecular Probes) was used to visualize nuclei; Live/Dead® red fixable (Molecular Probes) was used to visualize membrane. The cells were mounted with glycerol plastine and observed under a laser-scanning confocal microscope (LeicaSPS).

Results

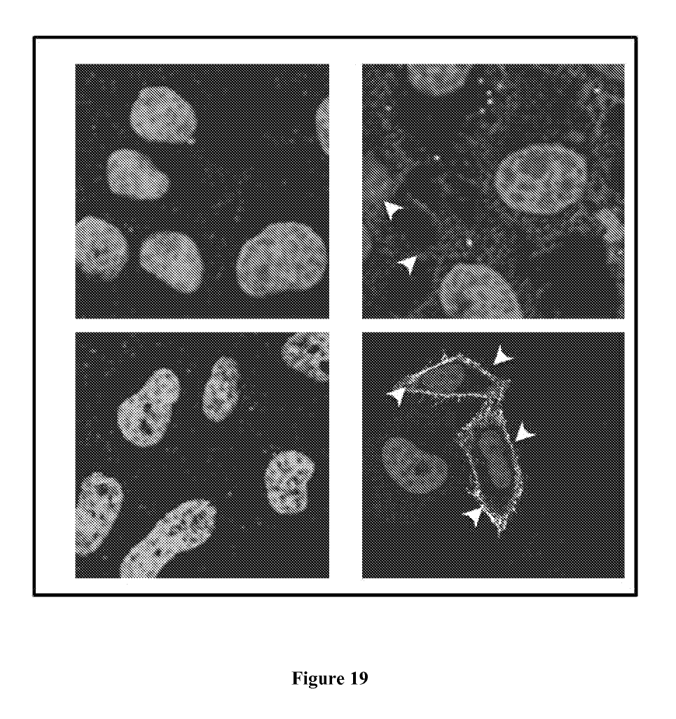

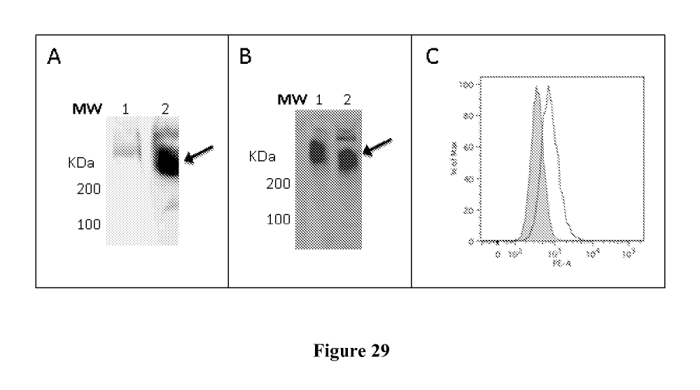

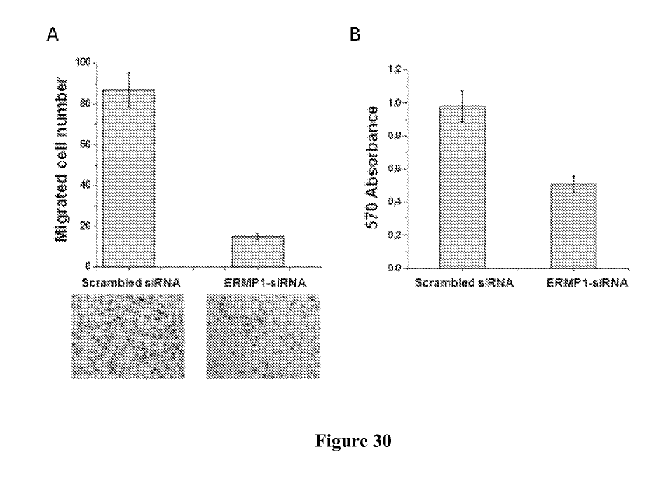

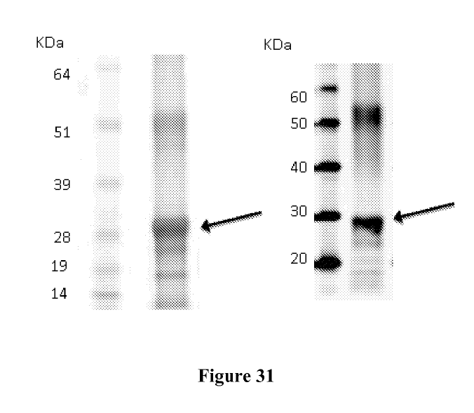

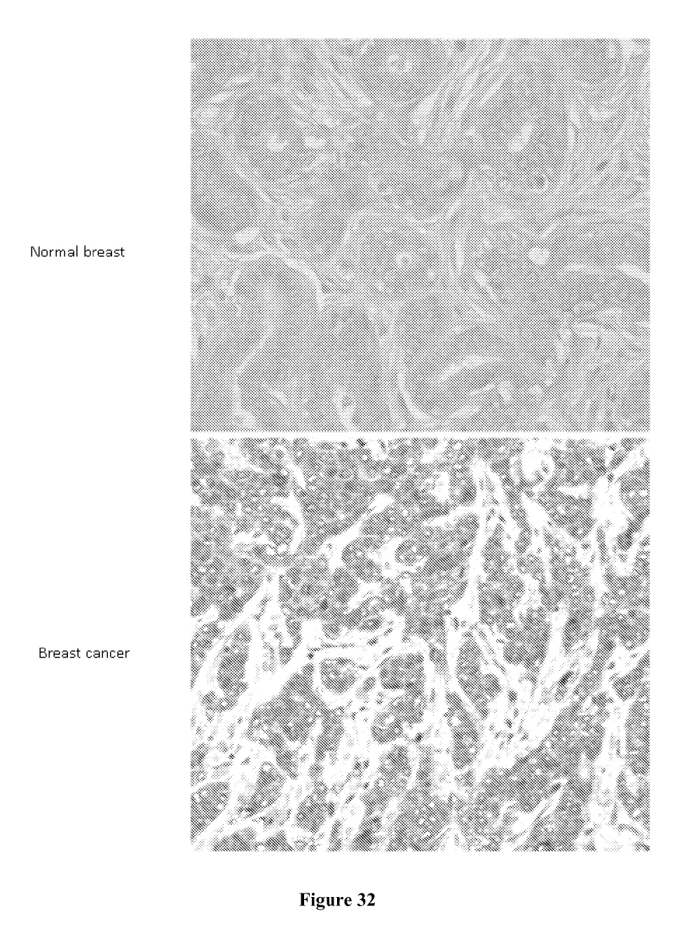

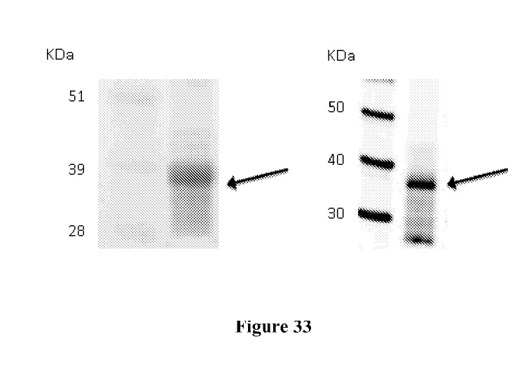

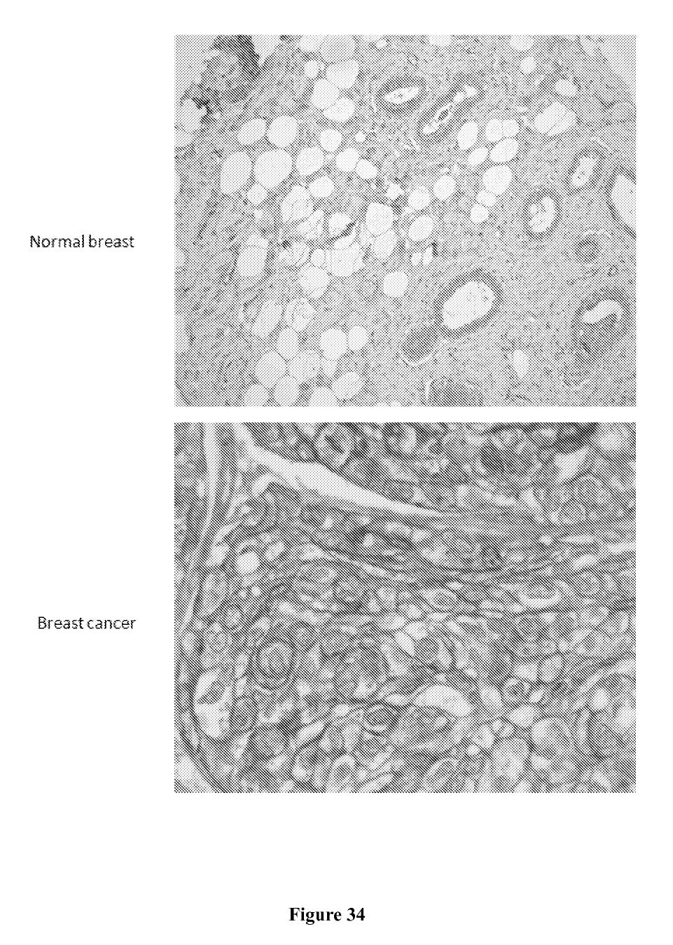

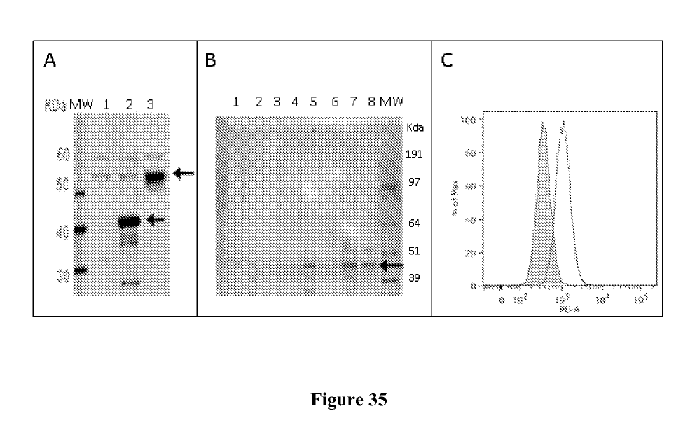

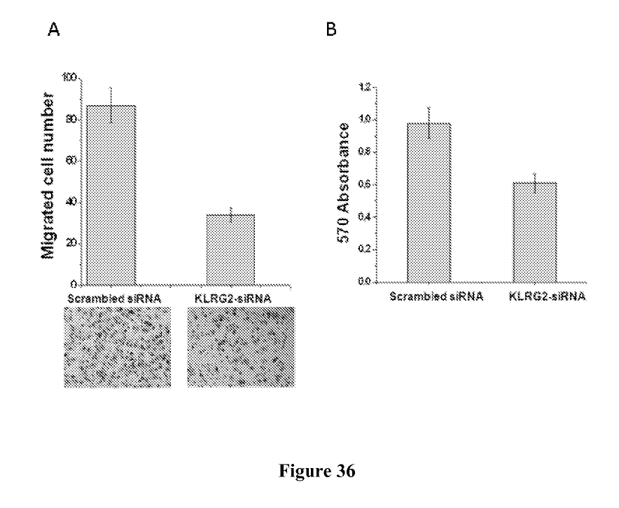

The selected coding sequences for C9orf46, SLC39A10, KLRG2 and ERMP1 were cloned in a eukaryotic expression vector and the derived plasmids were used for transient transfection of HeLa or HEK293T cells. Expression of target proteins Corf46 and KLRG2 was detected by Western blot in total protein extracts from HeLa, while expression of ERMP1 was analysed in trasnfected HEK-293T cells. Overall the data confirmed that the marker-specific antibodies recognized specifically their target proteins. Concerning C9orf46, a band of the expected size was visible in HeLa cells transfected with the C9orf46-expressing plasmid while the same band was either not visible or very faintly detected in HeLa cells transfected with the empty pcDNA3 plasmid (FIG. 5B). In the case of KLRG2, specific protein bands of expected size were detected in cells transfected with either of the two plasmids encoding the two annotated KLRG2 variants (FIG. 35A). As for cells transfected with ERMP1-encoding plasmid, a band of high molecular mass was specifically detected by the anti-ERMP1 antibody indicating that the protein forms stable aggregates (FIG. 29A). Expression of protein SLC3910 was carried by confocal microscopy of transfected cells. The anti-SLC39A10 specifically detected its target protein expressed by transfected cells, while no staining was visible in cell transfected with the empty pcDNA3 vector untransfected cells. In particular, the antibody mainly stained the surface of transfected cells (FIG. 19).

This indicates that this target protein is localized on the extracellular plasma membrane, accessible to the external environment.

### Example 4

### Detection of Target Protein in Tumor Tissue Homogenates

The presence of protein bands corresponding to the marker proteins was also investigated in tissue homogenates of breast tumor biopsies as compared to normal tissues from patients. Homogenates were prepared by mechanic tissue disruption in buffer containing 40 mM TRIS-HCl, 1 mM TCEP {Tris(2-carboxyethyl)-phosphine hydrochloride, Pierce} and 6M guanidine hydrochloride, pH 8. Western blot was performed by separation of the total protein extracts (20 μg/lane) proteins were detected by specific antibodies.

Results

An example of this type of experiments is represented for protein

C9orf46 and KLRG2. Antibodies specific C9orf46 and KLRG2 detected a specific protein band in breast tumor homogenates, while no or very faint bands were detected in normal breast homogenates, confirming the presence of the marker proteins in breast tumor. Results are reported in FIG. 5C and FIG. 35B.

### Example 5

### Expression of Target Protein in Tumor Cell Lines

Expression of target proteins was also assessed by WB and/or Flow cytometry on total extracts from breast tumor cell lines, including BT549, MCF7, MDA-MB231 and SKBR-3.

In each analysis, cells were cultured in under ATCC recommended conditions, and sub-confluent cell monolayers were detached with PBS-0.5 mM EDTA. For Western blot analysis, cells were lysed by several freeze-thaw passages in PBS-1% Triton. Total protein extracts were loaded on SDS-PAGE (2×10⁵ cells/lane), and subjected to WB with specific antibodies as described above.

For flow cytometry analysis, cells (2×10⁴ per well) were pelletted in 96 U-bottom microplates by centrifugation at 200×g for 5 min at 4° C. and incubated for 1 hour at 4° C. with the appropriate dilutions of the marker-specific antibodies. Cells were washed twice in PBS-5% FCS and incubated for 20 min with the appropriate dilution of R-Phycoerythrin (PE)-conjugated secondary antibodies (Jackson Immuno Research, PA, USA) at 4° C. After washing, cells were analysed by a FACS Canto II flow cytometer (Becton Dickinson). Data were analyzed with FlowJo 8.3.3 program.

Results

Example of the expression analysis is represented for C9orf46, DPY19L3, ERMP1, and SLC39A10.

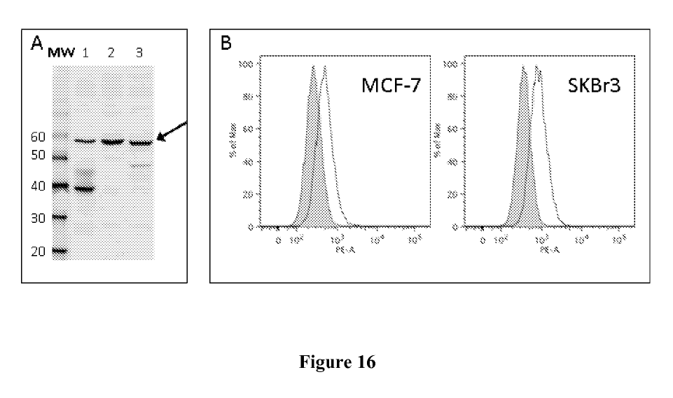

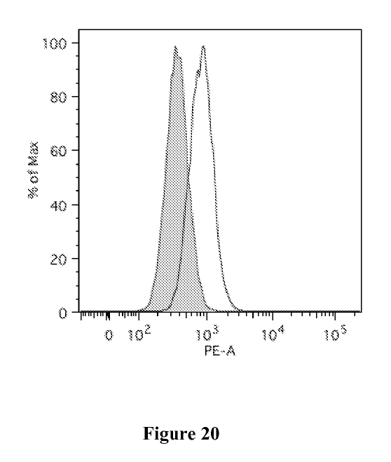

Western blot analysis of C9orf46 showed that a protein band of the expected sizes was detected in total protein extracts of breast tumor cell lines (BT549, MCF7), confirming its expression in breast tumor cell lines derived from breast tumor (FIG. 5A). Concerning ERMP1, both Western blot and flow cytometry analysis are represented. Western blot analysis shows a band of high molecular mass detected in the breast cell lines MCF7 and SKBR-3, showing an electrophoretic pattern similar to that reported in transfected cells (see Example 3). This further confirms the existence of stable aggregates for this protein confirming its expression in cell lines derived from breast tumor (FIG. 29B). Flow cytometry analysis indicates that ERMP1 is detected on the surface of the SKBR-3 cell line (FIG. 29C). As for DPY19L3, Western blot analysis showed a band of expected size in MCF7, MDA-MB231 and SKBr-3 was detected by the antibody o a panel of tumor cells lines (FIG. 16A). Flow cytometry analysis indicates that this protein is detected on the surface of MCF7 and SKBr-2 cell lines (FIG. 16B). Finally, expression and localization of SLC39A10 was analysed by flow cytometry. Results show that this protein is detected on the surface of the SKBR-3 cell line by the specific antibody (FIG. 20).

### Example 6

### Expression of the Marker Proteins Confers Malignant Cell Phenotype

To verify that the proteins included in the present invention can be exploited as targets for therapeutic applications, the effect of marker depletion was evaluated in vitro in cellular studies generally used to define the role of newly discovered proteins in tumor development. Marker-specific knock-down and control tumor cell lines were assayed for their proliferation and the migration/invasiveness phenotypes using the MTT and the Boyden in vitro invasion assay, respectively.

Method

Expression of marker genes were silenced in tumor cell lines by the siRNA technology and the influence of the reduction of marker expression on cell parameters relevant for tumor development was assessed in in vitro assays. The expression of marker genes was knocked down in a panel of epithelial tumor cell lines previously shown to express the tumor markers using a panel of marker-specific siRNAs (whose target sequences are reported in the Table II) using the HiPerfect transfection reagent (QIAGEN) following the manufacturer's protocol. As control, cells treated with irrelevant siRNA (scrambled siRNA) were analysed in parallel. At different time points (ranging from 24 to 72 hours) post transfection, the reduction of gene transcription was assessed by quantitative RT-PCR (Q-RT-PCR) on total RNA, by evaluating the relative marker transcript level, using the beta-actin, GAPDH or MAPK genes as internal normalization control. Afterwards, cell proliferation and migration/invasiveness assays were carried out to assess the effect of the reduced marker expression. Cell proliferation was determined using the MTT assay, a colorimetric assay based on the cellular conversion of a tetrazolium salt into a purple colored formazan product. Absorbance of the colored solution can be quantified using a spectrophotometer to provide an estimate of the number of attached living cells. Approximately 5×10³ cells/100 μl were seeded in 96-well plates in DMEM with 10% FCS to allow cell attachment. After overnight incubation with DMEM without FCS, the cells were treated with 2.5% FBS for 72 hours. Four hours before harvest 15 μL of the MTT dye solution (Promega) were added to each well. After 4-hour incubation at 37° C., the formazan precipitates were solubilized by the addition of 100 μL of solubilization solution (Promega) for 1 h at 37° C. Absorbance at 570 nm was determined on a multiwell plate reader (SpectraMax, Molecular Devices).

Cell migration/invasiveness was tested using the Boyden in vitro invasion assay, as compared to control cell lines treated with a scramble siRNA. This assay is based on a chamber of two medium-filled compartments separated by a microporous membrane. Cells are placed in the upper compartment and are allowed to migrate through the pores of the membrane into the lower compartment, in which chemotactic agents are present. After an appropriate incubation time, the membrane between the two compartments is fixed and stained, and the number of cells that have migrated to the lower side of the membrane is determined. For this assay, a transwell system, equipped with 8-μm pore polyvinylpirrolidone-free polycarbonate filters, was used. The upper sides of the porous polycarbonate filters were coated with 50 μg/cm² of reconstituted Matrigel basement membrane and placed into six-well culture dishes containing complete growth medium. Cells (1×10⁴ cells/well) were loaded into the upper compartment in serum-free growth medium. After 16 h of incubation at 37° C., non-invading cells were removed mechanically using cotton swabs, and the microporous membrane was stained with Diff-Quick solution. Chemotaxis was evaluated by counting the cells migrated to the lower surface of the polycarbonate filters (six randomly chosen fields, mean±SD).

Results

Examples of this analysis are reported for ERMP1 and KLRG2 in the breast tumor cell line MCF7. Gene silencing experiments with marker-specific siRNA reduced the marker transcripts (approximately 30-40 fold reduction), as determined by Q-RT_PCR. T II reports the sequences targeted by the siRNA molecules. The reduction of the expression of either of the two genes significantly impairs the proliferation and the invasiveness phenotypes of the MCF7 breast tumor cell line (FIGS. 30 and 36). This indicates that both proteins are involved in tumor development and are therefore likey targets for the development of anti-cancer therapies.

TABLE II

| NCBI gene | siRNA Target Sequence |
|---|---|
| KLRG2 | CGAGGACAATCTGGATATCAA |
| | CTGGAGCCCTCGAGCAAGAAA |
| ERMP1 | CCCGTGGTTCATCTGATATAA |
| | AAGGACTTTGCTCGGCGTTTA |
| | TACGTGGATGTTTGTAACGTA |
| | CTCGTATTGGCTCAATCATAA |

### REFERENCES

1) Anderson, L., and Seilhamer, J. (1997). A comparison of selected mRNA and protein abundances in human liver. Electrophoresis 18, 533-537;
2) Chen, G., Gharib, T. G., Wang, H., Huang, C. C., Kuick, R., Thomas, D. G., Shedden, K. A., Misek, D. E., Taylor, J. M., Giordano, T. J., Kardia, S. L., Iannettoni, M. D., Yee, J., Hogg, P. J., Orringer, M. B., Hanash, S. M., and Beer, D. G. (2003) Protein profiles associated with survival in lung adenocarcinoma. Proc. Natl. Acad. Sci. U.S. A 100, 13537-13542;
3) Ginestier, C., Charafe-Jauffret, E., Bertucci, F., Eisinger, F., Geneix, J., Bechlian, D., Conte, N., Adelaide, J., Toiron, Y., Nguyen, C., Viens, P., Mozziconacci, M. J., Houlgatte, R., Birnbaum, D., and Jacquemier, J. (2002) Distinct and complementary information provided by use of tissue and DNA microarrays in the study of breast tumor markers. Am. J. Pathol. 161, 1223-1233;
4) Gygi, S. P., Rochon, Y., Franza, B. R., and Aebersold, R. (1999) Correlation between protein and mRNA abundance in yeast. Mol. Cell. Biol. 19, 1720-1730; Nishizuka, S., Charboneau, L., Young, L., Major, S., Reinhold, W. C., Waltham, M., Kouros-Mehr, H., Bussey, K. J., Lee, J. K., Espina, V., Munson, P. J., Petricoin, E., III, Liotta, L. A., and Weinstein, J. N. (2003) Proteomic profiling of the NCl-60 cancer cell lines using new high-density reverse-phase lysate microarrays. Proc. Natl. Acad. Sci. U.S. A 100, 14229-14234;
5) Tyers, M., and Mann, M. (2003) From genomics to proteomics. Nature 422, 193-197;
6) Kagara N, Tanaka N, Noguchi S, Hirano T. (2007) Zinc and its transporter ZIP10 are involved in invasive behavior of breast cancer cells. Cancer Sci. 98:692-697;
7) Kasper G, Weiser A A, Rump A, Sparbier K, Dahl E, Hartmann A, Wild P, Schwidetzky U, Castaños-Vélez E, Lehmann K. (2005) Expression levels of the putative zinc transporter LIV-1 are associated with a better outcome of breast cancer patients. Int J. Cancer. 117:961-973;
8) Nguyen S T, Hasegawa S, Tsuda H, Tomioka H, Ushijima M, Noda M, Omura K, Miki Y, (2007) Identification of a predictive gene expression signature of cervical lymph node metastasis in oral squamous cell carcinoma., Cancer Sci. 98:740-746;
9) Garcia-Rudaz, C., Luna, F., Tapia, V., Kerr, B., Colgin, L., Galimi, F., Dissen, G. A., Rawlings, N. D. and Ojeda, S. R. (2007) Fxna, a novel gene differentially expressed in the rat ovary at the time of folliculogenesis, is required for normal ovarian histogenesis. Development. 134, 945-957;
10) Kratzschmar J, Haendler B, Eberspaecher U, Roosterman D, Donner P, Schleuning WD. (1996) The human cysteine-rich secretory protein (CRISP) family. Primary structure and tissue distribution of CRISP-1, CRISP-2 and CRISP-3. Eur J. Biochem. 236:827-836
11) Bjartell, A., Johansson, R., Bjork, T., adaleanu, V., Lundwall, A., Lilja, H., Kjeldsen, L. and Udby, L. (2006) Immunohistochemical detection of cysteine-rich secretory protein 3 in tissue and in serum from men with cancer or benign enlargement of the prostate gland, Prostate. 66: 591-603;
12) Bjartell, A. S., Al-Ahmadie, H., Serio, A. M., Eastham, J. A., Eggener, S. E., Fine, S. W., Udby, L., Gerald, W. L., Vickers, A. J., Lilja, H., Reuter, V. E. and Scardino, P. T. (2007), Association of cysteine-rich secretory protein 3 and beta-microseminoprotein with outcome after radical prostatectomy. Clin. Cancer Res. 13: 4130-4138.

1. A tumor marker, for use in the detection of breast cancer, which is selected from the group consisting of:
i) ERMP1, SEQ ID NO:73, SEQ ID NO:74, SEQ ID NO:75, or a different isoform having sequence identity of at least 80%, preferably at least 90%, more preferably at least 95% to SEQ ID NO:73, SEQ ID NO:74, SEQ ID NO:75, or a nucleic acid molecule containing a sequence coding for a ERMP1, protein, said encoding sequence being preferably selected from SEQ ID NO:76 SEQ ID NO 77 and SEQ ID NO:78;
ii) C6orf98 in one of its variant isoforms SEQ ID NO:1, or a different isoform having sequence identity of at least 80%, preferably at least 90%, more preferably at least 95% to SEQ ID NO:1; or a nucleic acid molecule containing a sequence coding for a C6orf98 protein, said encoding sequence being preferably SEQ ID NO: 2;
iii) C9orf46, in one of its variant isoforms SEQ ID NO:3, or a different isoform having sequence identity of at least 80%, preferably at least 90%, more preferably at least 95% to SEQ ID NO:3, or a nucleic acid molecule containing a sequence coding for a C9orf46 protein, said encoding sequence being preferably SEQ ID NO:4;
iv) FLJ37107, in one of its variant isoforms SEQ ID NO:5, or a different isoform having sequence identity of at least 80%, preferably at least 90%, more preferably at least 95% to SEQ ID NO:5; or a nucleic acid molecule containing a sequence coding for a FLJ37107 protein, said encoding sequence being preferably SEQ ID NO: 6;
v) YIPF2, SEQ ID NO:7, SEQ ID NO:8, or a different isoform having sequence identity of at least 80%, preferably at least 90%, more preferably at least 95% to SEQ ID NO:7 or SEQ ID NO:8, or a nucleic acid molecule containing a sequence coding for a YIPF2 protein, said encoding sequence being preferably selected from SEQ ID NO:9 and SEQ ID NO:10;
vi) UNQ6126, in one of its variant isoforms SEQ ID NO:11, or a different isoform having sequence identity of at least 80%, preferably at least 90%, more preferably at least 95% to SEQ ID NO:11, or a nucleic acid molecule containing a sequence coding for a UNQ6126 protein, said encoding sequence being preferably SEQ ID NO: 12;
vii) TRYX3, in one of its variant isoforms SEQ ID NO:13, or a different isoform having sequence identity of at least 80%, preferably at least 90%, more preferably at least 95% to SEQ ID NO:13, or a nucleic acid molecule containing a sequence coding for a TRYX3 protein, said encoding sequence being preferably SEQ ID NO:14;
viii) DPY19L3, in one of its variant isoforms SEQ ID NO:15, SEQ ID NO:16, SEQ ID NO:17, SEQ ID NO:18, or a different isoform having sequence identity of at least 80%, preferably at least 90%, more preferably at least 95% to any of SEQ ID NO:15, SEQ ID NO:16, SEQ ID NO:17 or SEQ ID NO:18, or a nucleic acid molecule containing a sequence coding for a DPY19L3 protein, said encoding sequence being preferably selected from SEQ ID NO:19, SEQ ID NO:20, SEQ ID NO:21, SEQ ID NO:22;
ix) SLC39A10, SEQ ID NO:23, SEQ ID NO:24 or a different isoform having sequence identity of at least 80%, preferably at least 90%, more preferably at least 95% to SEQ ID NO:23 or SEQ ID NO:24, or a nucleic acid molecule containing a sequence coding for a SLC39A10 protein, said encoding sequence being preferably selected from SEQ ID NO:25 and SEQ ID NO:26;
x) C14orf135, SEQ ID NO:27, SEQ ID NO:28, SEQ ID NO:29, SEQ ID NO:30, SEQ ID NO:31, or a different isoform having sequence identity of at least 80%, preferably at least 90%, more preferably at least 95% to any of SEQ ID NO:27, SEQ ID NO:28, SEQ ID NO:29, SEQ ID NO:30 or SEQ ID NO:31, or a nucleic acid molecule containing a sequence coding for a C14orf135 protein, said encoding sequence being preferably selected from SEQ ID NO:32, SEQ ID NO:33, SEQ ID NO:34, SEQ ID NO:35 and SEQ ID NO:36;
xi) DENND1B; in one of its variant isoforms SEQ ID NO:37, SEQ ID NO:38, SEQ ID NO:39, SEQ ID NO:40, or a different isoform having sequence identity of at least 80%, preferably at least 90%, more preferably at least 95% to any of SEQ ID NO:37, SEQ ID NO:38, SEQ ID NO:39 or SEQ ID NO:40, or a nucleic acid molecule containing a sequence coding for a DENND1B protein, said encoding sequence being preferably selected from SEQ ID NO:41, SEQ ID NO:42, SEQ ID NO:43 and SEQ ID NO:44;
xii) EMID1, in one of its variant isoforms SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, or a different isoform having sequence identity of at least 80%, preferably at least 90%, more preferably at least 95% to any of SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57 or SEQ ID NO:58, or a nucleic acid molecule containing a sequence coding for a DENND1B protein, said encoding sequence being preferably selected from SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71 and SEQ ID NO:72.
xiii) CRISP-3, SEQ ID NO: 79, SEQ ID NO:80, SEQ ID NO:81, or a different isoform having sequence identity of at least 80%, preferably at least 90%, more preferably at least 95% to SEQ ID NO: 79, SEQ ID NO:80, SEQ ID NO:81, or a nucleic acid molecule containing a sequence coding for a CRISP-3, protein, said encoding sequence being preferably selected from SEQ ID NO:82, SEQ ID NO:83 and SEQ ID NO:84.
xiv) KLRG2, SEQ ID: NO 85, SEQ ID NO:86 or a different isoform having sequence identity of at least 80%, preferably at least 90%, more preferably at least 95% to SEQ ID: NO 85 or SEQ ID: NO 86, or a nucleic acid molecule containing a sequence coding for a KLRG2 protein, said encoding sequence being preferably selected from SEQ ID NO: 87 and SEQ ID NO: 88;

2. A method of screening a tissue sample for malignancy, said method comprising determining the presence in said sample of at least one of the tumor markers according to claim 1 or a combination thereof.

3. A method according to claim 2, wherein the tissue sample is a sample of breast tissue.

4. A method according to claim 2, wherein the tumor marker is a protein, said method being based on immunoradiometric, immunoenzymatic or immunohistochemical techniques.

5. A method according to claim 2, wherein the tumor marker is a nucleic acid molecule, said method being based on polymerase chain reaction techniques.

6. A method in vitro for determining the presence of a breast tumor in a subject, which comprises the steps of:
(a) providing a sample of the tissue suspected of containing tumor cells;
(b) determining the presence of a tumor marker according to claim 1 or a combination thereof as per claim 2 in said tissue sample by detecting the expression of the marker protein or the presence of the respective mRNA transcript;
wherein the detection of one or more tumor markers in the tissue sample is indicative of the presence of tumor in said subject.

7. A method of screening a test compound as an antitumor candidate, which comprises contacting cells expressing a tumor marker protein according to claim 1 with the test compound, and determining the binding of said compound to said tumor marker.

8. An antibody or a fragment thereof which is able to specifically recognize and bind to one of the tumor marker proteins according to claim 1.

9. An antibody according to claim 8, which is either monoclonal or polyclonal.

10. (canceled)

11. (canceled)

12. A siRNA molecule having a sequence complementary to one of SEQ ID NOs:89 through SEQ ID NO:94, for use in tumor-gene silencing.
